(19) United States
(12) Patent Application Publication
(10) Pub. No.: US 2010/0166765 A1
(43) Pub. Date: Jul. 1, 2010

(54) BENZOQUINAZOLINE DERIVATIVES

(76) Inventors: Sandra Ammon, Basel (CH); Rene Beerli, Binningen (CH); Leo Widler, Muenchenstein (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/063,526

(22) PCT Filed: Aug. 14, 2006
(86) PCT No.: PCT/EP2006/008036

§ 371 Date: Apr. 7, 2008

(30) Foreign Application Priority Data
Aug. 15, 2005 (GB) 0516723.4

(51) Int. Cl. A61K 31/517; C07D 239/72; C07D 403/12; A61P 19/08; C07D 237/30; A61K 31/502; A61K 33/06; A61K 38/23; A61K 31/56; A61K 39/395; A61K 31/59

(52) U.S. Cl. 424/158.1; 544/283; 514/266.1; 514/266.24; 544/284; 514/266.2; 514/266.21; 514/266.22; 544/237; 514/248; 424/682; 514/12; 514/171; 514/167

(57) ABSTRACT

A compound of formula (I) or a pharmaceutically acceptable salt or prodrug ester thereof, wherein the groups R2, R3, R4, Q, X and Y are as defined in the specification, is useful in the treatment of bone conditions related to increased calcium depletion or resorption.

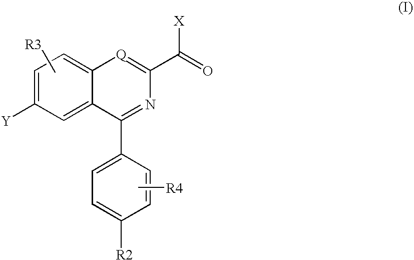

The present invention relates to bicyclic compounds, in particular to 2-benzoquinazoline derivatives and to pharmaceutical uses thereof.

Accordingly the invention provides a compound of formula (I) or a pharmaceutically acceptable salt or prodrug ester thereof:

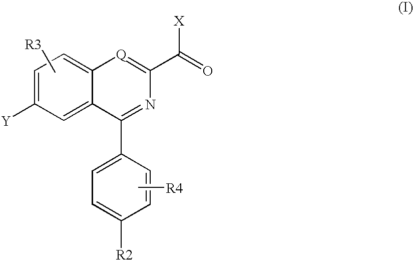

wherein:

Q is CH or N;

R2 is C₁-C₄ alkyl;

Y is selected from the group consisting of: R5-O—, C₁-C₄ alkyl, C₁-C₄ alkenyl, C₁-C₄ alkynyl, R5-NH—;

where R5 is C₁-C₄ alkyl, C₁-C₄ alkenyl, C₁-C₄ alkynyl;

X is selected from the group consisting of aryl, heteroaryl, C₁-C₁₀ alkyl, C₁-C₁₀ alkyloxy, cycloalkyl, heterocycloalkyl, aryl C₁-C₄ alkyl, heteroaryl C₁-C₄ alkyl, cycloalkyl C₁-C₄ alkyl, heterocycloalkyl C₁-C₄ alkyl, arylamino, heteroarylamino, aryl C₁-C₄ alkylamino, heteroaryl C₁-C₄ alkylamino, C₁-C₆ alkylamino, C₁-C₆ dialkylamino, aryloxy, heteroaryloxy, aryl C₁-C₄ alkyloxy, heteroaryl C₁-C₄ alkyloxy, cycloalkyl C₁-C₄ alkylamino, heterocycloalkyl C₁-C₄ alkylamino, cycloalkyl C₁-C₄ alkyloxy or heterocycloalkyl C₁-C₄ alkyloxy each of which is optionally substituted once or more;

the optional substituent or substituents on X being independently selected from the group consisting of halo, cyano, trifluoromethyl, nitro, hydroxy, optionally substituted (C₁-C₄ alkyl, C₁-C₄ alkyloxy, amino, sulfanyl, sulfonyl, amino, oxycarbonyl, hydroxyl, sulfinyl, aminosulfonyl, sulfonylamino, carbonyl, carbonyloxy, carbonyl amino, carboxyl, acyl, acylamino, or carbamoyl); the optional substituent or substituents being selected from C₁-C₆ alkyl, C₁-C₆ alkyloxy, carboxyl, hydroxyl, hydroxy C₁-C₄ alkyl; each of which in turn may be optionally substituted by C₁-C₆ alkyloxy, C₁-C₆ alkyl, C₁-C₃ fluorinated alkyl, C₁-C₆ alkyloxy, carboxyl, hydroxyl, hydroxy C₁-C₄ alkyl, halo, cyano, nitro.

R3 and R4 each represent one or more substituents independently selected from: H, halo, C₁-C₄ alkyl, C₁-C₄ alkyloxy, CF₃;

the optional substituent or substituents on R3 or R4 being independently selected from the group consisting of C₁-C₄ alkyl, halo, C₁-C₄ alkyloxy, cyano, sulfanyl, sulfonyl, amino, oxycarbonyl, hydroxyl which may in turn be optionally substituted once or more by C₁-C₄ alkyl, halo, C₁-C₄ alkyloxy, cyano, sulfanyl, sulfonyl, amino, oxycarbonyl or hydroxyl.

In a second aspect, the present invention provides a compound of formula (I′) or a pharmaceutically acceptable salt or prodrug ester thereof:

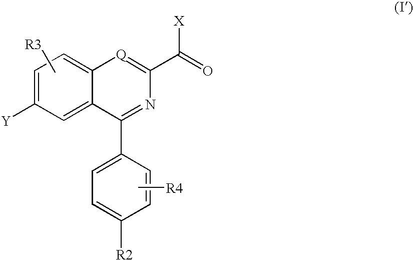

wherein:

Q is CH or N;

R2 is C₁-C₄ alkyl;

Y is selected from the group consisting of: R5-O—, C₁-C₄ alkyl, C₁-C₄ alkenyl, C₁-C₄ alkynyl, R5-NH—;

where R5 is selected from C₁-C₄ alkyl, C₁-C₄ alkenyl, C₁-C₄ alkynyl;

X is selected from the group consisting of aryl, heteroaryl, C₁-C₆ alkyl, cycloalkyl, heterocycloalkyl, aryl C₁-C₄ alkyl, heteroaryl C₁-C₄ alkyl, arylamino, aryl C₁-C₄ alkylamino, heteroaryl C₁-C₄ alkylamino, C₁-C₆ alkylamino, C₁-C₆ dialkylamino amino, aryloxy, heteroaryloxy, aryl C₁-C₄ alkyloxy, or heteroaryl C₁-C₄ alkyloxy, each of which is optionally substituted once or more;

the optional substituent or substituents on X being independently selected from the group consisting of C₁-C₄ alkyl, halo, C₁-C₄ alkyloxy, cyano, trifluoromethyl, hydroxy, amino, nitro, alkyl, lower alkyl substituted (sulfanyl, sulfonyl, amino, oxycarbonyl, hydroxyl, sulfinyl, carbonyl, carboxyl, carbamoyl or aminoacyl);

R3 and R4 each represent one or more substituents independently selected from: H, halo, optionally substituted C₁-C₄ alkyl, optionally substituted C₁-C₄ alkyloxy;

the optional substituent or substituents on R3 or R4 being independently selected from the group consisting of C₁-C₄ alkyl, halo, C₁-C₄ alkyloxy, cyano, sulfanyl, sulfonyl, amino, oxycarbonyl, hydroxyl.

With reference to the compounds of formula (I) and (I′), Q is preferably N.

R2 is preferably isopropyl, cyclopropyl or t-butyl. More preferably, R2 is isopropyl. Alternatively, R2 is preferably cyclopropyl.

R3 and R4 are preferably halo or H. More preferably, R3 and R4 are H.

Y is preferably R5-O—. More preferably, R5 is propargyl.

Preferably X is optionally substituted (aryl, heteroaryl, arylamino, heteroarylamino, aryl C₁-C₄ alkylamino, heteroaryl C₁-C₄ alkylamino, aryloxy, heteroaryloxy, C₁-C₆ alkyloxy, aryl C₁-C₄ alkyloxy or heteroaryl C₁-C₄ alkyloxy). More preferably, X is optionally substituted (aryl, heteroaryl, arylamino, heteroarylamino, aryl C₁-C₄ alkylamino, heteroaryl C₁-C₄ alkylamino, aryloxy, C₁-C₆ alkyloxy or aryl C₁-C₄ alkyloxy). Alternatively preferably X is optionally substituted (aryl, heteroaryl or heterocycloalkyl). Alternatively preferably X is optionally substituted aryl, preferably phenyl or naphthalenyl. More preferably, X is optionally substituted phenyl. Alternatively, X is optionally substituted heteroaryl. Preferred heteroaryl groups are furanyl, thienyl, pyrrolyl, thiazolyl, benzothiazolyl, pyridinyl and benz[b]thiophen-2-yl. Alternatively preferably, X is arylamino or heteroarylamino. Preferred arylamino and heteroarylamino groups are pyridinylamino, pyrazolylamino, thioazolylamino, naphthalenylamino, quinolinaylamino, isoquinolinaylamino, phthalazinylamino, benzoimidazolylamino and benzothiazolylamino. Alternatively preferably, X is aryloxy, C₁-C₆ alkyloxy or aryl C₁-C₄ alkyloxy. Alternatively preferably, X is optionally substituted heterocycloalkyl. A preferred heterocycloalkyl substituent is piperidinyl.

Alternatively, X is preferably optionally substituted (aryl, heteroaryl, cycloalkyl or heterocycloalkyl). More preferably, X is optionally substituted phenyl. Yet more preferably, X is a phenyl group substituted in the ortho- or para-position. Alternatively preferably, X is a heteroaryl which is optionally substituted. Alternatively preferably, X is optionally substituted arylamino. More preferably, X is substituted arylamino containing substituent at the meta position.

A third aspect of the invention provides a compound having the formula (II) or a pharmaceutically acceptable salt or prodrug ester thereof:

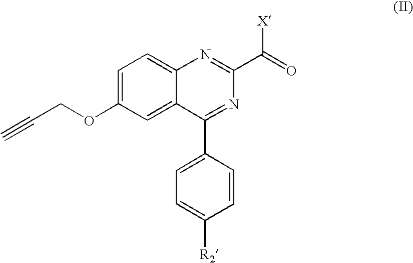

wherein:

X′ is selected from the group consisting of aryl, heteroaryl, cycloalkyl, heterocycloalkyl, —C₁-C₄ alkylaryl, —C₁-C₄ alkylheteroaryl, arylamino, heteroarylamino, aryl C₁-C₄ alkylamino, heteroaryl C₁-C₄ alkylamino, aryloxy, heteroaryloxy, aryl C₁-C₄ alkyloxy, heteroaryl C₁-C₄ alkyloxy, aryl C₁-C₄ alkyl, heteroaryl C₁-C₄ alkyl, C₁-C₆ alkyl, —C₁-C₄alkylamino or amino, each of which is optionally substituted once or more;

the optional substituent or substituents on X′ being independently selected from the group consisting of halo, cyano, trifluoromethyl, nitro, hydroxy, optionally substituted (C₁-C₄ alkyl, C₁-C₄ alkyloxy, amino, sulfanyl, sulfonyl, amino, oxycarbonyl, hydroxyl, sulfinyl, carbonyl, carboxyl, acyl, acylamino, carbamoyl or aminoacyl); the optional substituent or substituents being selected from C₁-C₆ alkyl, C₁-C₆ alkyloxy, carboxyl, hydroxyl, hydroxy C₁-C₄ alkyl; each of which in turn may be optionally substituted by C₁-C₆ alkyloxy, C₁-C₆ alkyl, C₁-C₆ alkyloxy, carboxyl, hydroxyl, hydroxy C₁-C₄ alkyl, halo, cyano, nitro.

R₂′ is C₁-C₄ alkyl.

A fourth aspect of the invention provides a compound of formula (II) or a pharmaceutically acceptable salt or prodrug ester thereof:

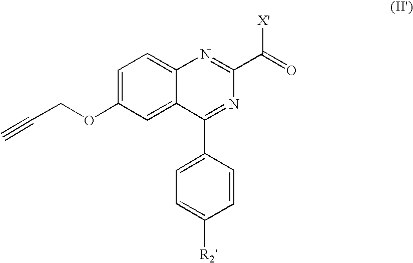

wherein:

X′ is selected from the group consisting of aryl, heteroaryl, cycloalkyl, heterocycloalkyl, aryl C₁-C₄ alkyl, heteroaryl C₁-C₄ alkyl, aryl C₁-C₄ alkylamino or heteroaryl C₁-C₄ alkylamino, each of which is optionally substituted once or more;

the optional substituent or substituents on X′ being independently selected from the group consisting of C₁-C₄ alkyl, halo, C₁-C₄ alkyloxy, cyano, trifluoromethyl, hydroxy, amino, nitro, alkyl, lower alkyl substituted (sulfanyl, sulfonyl, amino, oxycarbonyl, hydroxyl, sulfinyl, carbonyl, carboxyl, carbamoyl or aminoacyl);

R₂′ is C₁-C₄ alkyl.

With reference to compounds of formula (II) and (II′), preferably R₂′ is isopropyl, t-butyl or cyclopropyl. More preferably, R₂′ is isopropyl. Alternatively, R₂′ is preferably cyclopropyl.

Preferably X′ is optionally substituted (aryl, heteroaryl, arylamino, heteroarylamino, aryl C₁-C₄ alkylamino, heteroaryl C₁-C₄ alkylamino, aryloxy, heteroaryloxy, C₁-C₆ alkyloxy, aryl C₁-C₄ alkyloxy or heteroaryl C₁-C₄ alkyloxy). More preferably, X′ is optionally substituted (aryl, heteroaryl, arylamino, heteroarylamino, aryl C₁-C₄ alkylamino, heteroaryl C₁-C₄ alkylamino, aryloxy, C₁-C₆ alkyloxy or aryl C₁-C₄ alkyloxy). Alternatively preferably X′ is optionally substituted (aryl, heteroaryl or heterocycloalkyl). Alternatively preferably X′ is optionally substituted aryl, preferably phenyl or naphthalenyl. More preferably, X′ is optionally substituted phenyl. Alternatively, X′ is optionally substituted heteroaryl. Preferred heteroaryl groups are furanyl, thienyl, pyrrolyl, thiazolyl, benzothiazolyl, pyridinyl and benz[b]thiophen-2-yl. Alternatively preferably, X′ is arylamino or heteroarylamino. Preferred heteroarylamino groups are pyridinylamino, pyrazolylamino, thioazolylamino, naphthalenylamino, quinolinaylamino, isoquinolinaylamino, phthalazinylamino, benzoimidazolylamino and benzothiazolylamino. Alternatively preferably, X′ is aryloxy, C₁-C₆ alkyloxy or aryl C₁-C₄ alkyloxy. Alternatively preferably, X′ is optionally substituted heterocycloalkyl.

For the avoidance of doubt, the terms listed below are to be understood to have the following meaning throughout the present description and claims:

The term “lower”, when referring to organic radicals or compounds means a compound or radical with may be branched or unbranched with up to and including 7 carbon atoms.

A lower alkyl group may be branched, unbranched or cyclic and contains 1 to 7 carbon atoms, preferably 1 to 4 carbon atoms. Lower alkyl represents, for example: methyl, ethyl, propyl, butyl, isopropyl, isobutyl, tertiary butyl or 2,2-dimethylpropyl.

A lower alkoxy group may be branched or unbranched and contains 1 to 7 carbon atoms, preferably 1 to 6 carbon atoms. Lower alkoxy represents, for example: methoxy, ethoxy, propoxy, butoxy, isopropoxy, isobutoxy or tertiary butoxy. Lower alkoxy includes cycloalkyloxy and cycloalkyl-lower alkyloxy.

A lower alkene, alkenyl or alkenoxy group is branched or unbranched and contains 2 to 7 carbon atoms, preferably 1 to 4 carbon atoms and contains at least one carbon-carbon double bond. Lower alkene, lower alkenyl or lower alkenyloxy represents for example vinyl, prop-1-enyl, allyl, butenyl, isopropenyl or isobutenyl and the oxy equivalents thereof.

A lower akyne or alkynyl group is branched or unbranched and contains 2 to 7 carbon atoms, preferably 1 to 4 carbon atoms and contains at least one carbon-carbon triple bond. Lower alkyne or lower alkynyl or lower alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo.

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one or two heteroatoms. Heterocyclic aryl represents, for example: pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl. Heterocyclic aryl also includes such substituted radicals.

Cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. The term heterocycloalkyl is intended also to include bridged heterocycloalkyl groups such as 3-hyroxy-8-aza-bicyclo[3.2.1]oct-8-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Preferred compounds of formula (I) are:

(4-tert-Butyl-phenyl)-[4-(4-isopropyl-phenyl)-6-propargyloxy-quinazolin-2-yl]-methanone

[4-(4-Isopropyl-phenyl)-6-propargyloxy-quinazolin-2-yl]-phenyl-methanone

(2-Methoxy-phenyl)-[4-(4-Isopropyl-phenyl)-6-propargyloxy-quinazolin-2-yl]-methanone

(3-Methoxy-phenyl)-[4-(4-Isopropyl-phenyl)-6-propargyloxy-quinazolin-2-yl]-methanone

(4-Methoxy-phenyl)-[4-(4-Isopropyl-phenyl)-6-propargyloxy-quinazolin-2-yl]-methanone

(4-Fluoro-phenyl)-[4-(4-isopropyl-phenyl)-6-propargyloxy-quinazolin-2-yl]-methanone

(3-Fluoro-phenyl)-[4-(4-isopropyl-phenyl)-6-propargyloxy-quinazolin-2-yl]-methanone

(3-Chloro-phenyl)-[4-(4-isopropyl-phenyl)-6-propargyloxy-quinazolin-2-yl]-methanone

(4-Chloro-phenyl)-[4-(4-isopropyl-phenyl)-6-propargyloxy-quinazolin-2-yl]-methanone

(4-Fluoro-phenyl)-[4-(4-tert.butyl-phenyl)-6-propargyloxy-quinazolin-2-yl]-methanone

(3-Fluoro-phenyl)-[4-(4-tert.butyl-phenyl)-6-propargyloxy-quinazolin-2-yl]-methanone

(3-Bromo-phenyl)-[4-(4-isopropyl-phenyl)-6-propargyloxy-quinazolin-2-yl]-methanone

(4-Bromo-phenyl)-[4-(4-isopropyl-phenyl)-6-propargyloxy-quinazolin-2-yl]-methanone

(4-Methyl-phenyl)-[4-(4-isopropyl-phenyl)-6-propargyloxy-quinazolin-2-yl]-methanone

(4-Isopropyl-phenyl)-[4-(4-isopropyl-phenyl)-6-propargyloxy-quinazolin-2-yl]-methanone

(4-Ethyl-phenyl)-[4-(4-isopropyl-phenyl)-6-propargyloxy-quinazolin-2-yl]-methanone

(4-Propyl-phenyl)-[4-(4-isopropyl-phenyl)-6-propargyloxy-quinazolin-2-yl]-methanone

(4-Cyano-phenyl)-[4-(4-isopropyl-phenyl)-6-propargyloxy-quinazolin-2-yl]-methanone

(4-Methylthio-phenyl)-[4-(4-isopropyl-phenyl)-6-propargyloxy-quinazolin-2-yl]-methanone

(4-Methansulfonyl-phenyl)-[4-(4-isopropyl-phenyl)-6-propargyloxy-quinazolin-2-yl]-methanone

(4-Dimethylamino-phenyl)-[4-(4-isopropyl-phenyl)-6-propargyloxy-quinazolin-2-yl]-methanone

(4-Ethoxy-phenyl)-[4-(4-isopropyl-phenyl)-6-propargyloxy-quinazolin-2-yl]-methanone

4-[4-(4-Isopropyl-phenyl)-6-propargyloxy-quinazoline-2-carbonyl]-benzoic acid methyl ester

(4-Dimethylamino-phenyl)-[4-(4-tert.butyl-phenyl)-6-propargyloxy-quinazolin-2-yl]-methanone

(4-Dimethylamino-phenyl)-[4-(4-cyclopropyl-phenyl)-6-propargyloxy-quinazolin-2-yl]-methanone

4-[4-(4-Isopropyl-phenyl)-6-propargyloxy-quinazoline-2-carbonyl]-benzoic acid ethyl ester

(4-Methoxy-phenyl)-[4-(4-tert.butyl-phenyl)-6-propargyloxy-quinazolin-2-yl]-methanone

(4-Ethoxy-phenyl)-[4-(4-cyclopropyl-phenyl)-6-propargyloxy-quinazolin-2-yl]-methanone

(3-Ethoxy-4-methoxy-phenyl)-[4-(4-isopropyl-phenyl)-6-propargyloxy-quinazolin-2-yl]-methanone

(4-tert.Butyloxy-phenyl)-[4-(4-isopropyl-phenyl)-6-propargyloxy-quinazolin-2-yl]-methanone

(4-Hydroxy)-[4-(4-isopropyl-phenyl)-6-propargyloxy-quinazolin-2-yl]-methanone

(4-Butyloxy)-[4-(4-isopropyl-phenyl)-6-propargyloxy-quinazolin-2-yl]-methanone

Furan-2-yl-[4-(4-isopropyl-phenyl)-6-propargyloxy-quinazolin-2-yl]-methanone

Furan-3-yl-[4-(4-tert.butyl-phenyl)-6-propargyloxy-quinazolin-2-yl]-methanone

Furan-3-yl-[4-(4-isopropyl-phenyl)-6-propargyloxy-quinazolin-2-yl]-methanone

Thiophen-2-yl-[4-(4-isopropyl-phenyl)-6-propargyloxy-quinazolin-2-yl]-methanone

(3-Methyl-thiophen-2-yl-[4-(4-isopropyl-phenyl)-6-propargyloxy-quinazolin-2-yl]-methanone

Benz[b]thiophen-2-yl-[4-(4-isopropyl-phenyl)-6-propargyloxy-quinazolin-2-yl]-methanone

Thiophen-3-yl-[4-(4-isopropyl-phenyl)-6-propargyloxy-quinazolin-2-yl]-methanone

(1-Methyl-1H-pyrrol)-2-yl-[4-(4-isopropyl-phenyl)-6-propargyloxy-quinazolin-2-yl]-methanone

4-(4-Isopropyl-phenyl)-6-propargyloxy-quinazoline-2-carboxylic acid ethyl ester

[4-(4-isopropyl-phenyl)-6-propargyloxy-quinazolin-2-yl]-pyridine-3-yl-methanone

[4-(4-Isopropyl-phenyl)-6-propargyloxy-quinazolin-2-yl]-naphthalen-1-yl-methanone

[4-(4-Isopropyl-phenyl)-6-propargyloxy-naphathalen-2-yl]-methanone

Benzothiazol-2-yl-[4-(4-isopropyl-phenyl)-6-propargyloxy-quinazolin-2-yl]-methanone

[4-(4-Isopropyl-phenyl)-6-propargyloxy-quinazolin-2-yl]-thiazol-5-yl-methanone

[4-(4-Isopropyl-phenyl)-6-propargyloxy-quinazolin-2-yl]-piperidin-1-yl-methanone

4-(4-Isopropyl-phenyl)-6-prop-2-ynyloxy-quinazoline-2-carboxylic acid (3-chloro-phenyl)-amide

4-(4-Isopropyl-phenyl)-6-prop-2-ynyloxy-quinazoline-2-carboxylic acid (3-methoxy-phenyl)-amide

4-(4-Isopropyl-phenyl)-6-prop-2-ynyloxy-3,4-dihydro-quinazoline-2-carboxylic acid (3-methylsulfanyl-phenyl)-amide

4-(4-Isopropyl-phenyl)-6-prop-2-ynyloxy-3,4-dihydro-quinazoline-2-carboxylic acid (3-methanesulfonyl-phenyl)-amide

4-(4-Isopropyl-phenyl)-6-prop-2-ynyloxy-quinazoline-2-carboxylic acid (3-trifluoromethylsulfanyl-phenyl)-amide

4-(4-Isopropyl-phenyl)-6-prop-2-ynyloxy-3,4-dihydro-quinazoline-2-carboxylic acid (3-sulfamoyl-phenyl)-amide

4-(4-Isopropyl-phenyl)-6-prop-2-ynyloxy-3,4-dihydro-quinazoline-2-carboxylic acid [3-(2-hydroxy-ethanesulfonyl)-phenyl]-amide

4-(4-Isopropyl-phenyl)-6-prop-2-ynyloxy-3,4-dihydro-quinazoline-2-carboxylic acid (5-ethanesulfonyl-2-hydroxy-phenyl)-amide

4-(4-Isopropyl-phenyl)-6-prop-2-ynyloxy-quinazoline-2-carboxylic acid (3-nitro-phenyl)-amide

4-(4-Isopropyl-phenyl)-6-prop-2-ynyloxy-quinazoline-2-carboxylic acid (3-cyano-phenyl)-amide

3-{[4-(4-Isopropyl-phenyl)-6-prop-2-ynyloxy-quinazoline-2-carbonyl]-amino}-benzoic acid methyl ester

3-{[4-(4-Isopropyl-phenyl)-6-prop-2-ynyloxy-quinazoline-2-carbonyl]-amino}-benzoic acid ethyl ester

3-{[4-(4-Isopropyl-phenyl)-6-prop-2-ynyloxy-quinazoline-2-carbonyl]-amino}-benzoic acid isopropyl ester

3-{[4-(4-Isopropyl-phenyl)-6-prop-2-ynyloxy-quinazoline-2-carbonyl]-amino}-benzoic acid tert-butyl ester

4-(4-Isopropyl-phenyl)-6-prop-2-ynyloxy-quinazoline-2-carboxylic acid (3-carbamoyl-phenyl)-amide

4-(4-Isopropyl-phenyl)-6-prop-2-ynyloxy-quinazoline-2-carboxylic acid (3-acetyl-phenyl)-amide

3-{[4-(4-Isopropyl-phenyl)-6-prop-2-ynyloxy-quinazoline-2-carbonyl]-amino}-5-methoxy-benzoic acid methyl ester

4-(4-Isopropyl-phenyl)-6-prop-2-ynyloxy-quinazoline-2-carboxylic acid (3-methylcarbamoyl-phenyl)-amide

4-(4-Isopropyl-phenyl)-6-prop-2-ynyloxy-quinazoline-2-carboxylic acid (3-tert-butylcarbamoyl-phenyl)-amide

4-(4-Isopropyl-phenyl)-6-prop-2-ynyloxy-quinazoline-2-carboxylic acid (3-dimethylcarbamoyl-5-trifluoromethyl-phenyl)-amide

3-{[4-(4-Isopropyl-phenyl)-6-prop-2-ynyloxy-quinazoline-2-carbonyl]-amino}-5-trifluoromethyl-benzoic acid methyl ester

3-{[4-(4-Isopropyl-phenyl)-6-prop-2-ynyloxy-quinazoline-2-carbonyl]-amino}-5-trifluoromethyl-benzoic acid isopropyl ester

2-Fluoro-5-{[4-(4-isopropyl-phenyl)-6-prop-2-ynyloxy-quinazoline-2-carbonyl]-amino}-benzoic acid methyl ester

2-Fluoro-5-{[4-(4-isopropyl-phenyl)-6-prop-2-ynyloxy-quinazoline-2-carbonyl]-amino}-benzoic acid isopropyl ester

2-Chloro-5-{[4-(4-isopropyl-phenyl)-6-prop-2-ynyloxy-quinazoline-2-carbonyl]-amino}-benzoic acid methyl ester

2,5-Dichloro-3-{[4-(4-isopropyl-phenyl)-6-prop-2-ynyloxy-quinazoline-2-carbonyl]-amino}-benzoic acid methyl ester

2,5-Dichloro-3-{[4-(4-isopropyl-phenyl)-6-prop-2-ynyloxy-quinazoline-2-carbonyl]-amino}-benzoic acid isopropyl ester

4-(4-Isopropyl-phenyl)-6-prop-2-ynyloxy-quinazoline-2-carboxylic acid (3-cyano-5-fluoro-phenyl)-amide

4-(4-Isopropyl-phenyl)-6-prop-2-ynyloxy-quinazoline-2-carboxylic acid (3,4-dicyano-phenyl)-amide

4-(4-Isopropyl-phenyl)-6-prop-2-ynyloxy-quinazoline-2-carboxylic acid (4-cyano-3-trifluoromethyl-phenyl)-amide

4-(4-Isopropyl-phenyl)-6-prop-2-ynyloxy-quinazoline-2-carboxylic acid (3-trifluoromethyl-phenyl)-amide

4-(4-Isopropyl-phenyl)-6-prop-2-ynyloxy-quinazoline-2-carboxylic acid (4-acetylamino-3-trifluoromethyl-phenyl)-amide

4-(4-Isopropyl-phenyl)-6-prop-2-ynyloxy-quinazoline-2-carboxylic acid (3-methoxy-5-trifluoromethyl-phenyl)-amide

4-(4-Isopropyl-phenyl)-6-prop-2-ynyloxy-quinazoline-2-carboxylic acid (3,5-bis-trifluoromethyl-phenyl)-amide

4-(4-Isopropyl-phenyl)-6-prop-2-ynyloxy-quinazoline-2-carboxylic acid (3-fluoro-5-trifluoromethyl-phenyl)-amide

4-(4-Isopropyl-phenyl)-6-prop-2-ynyloxy-quinazoline-2-carboxylic acid (4-fluoro-3-trifluoromethyl-phenyl)-amide

3-{[4-(4-Isopropyl-phenyl)-6-prop-2-ynyloxy-quinazoline-2-carbonyl]-amino}-2-methyl-benzoic acid methyl ester

3-{[4-(4-Isopropyl-phenyl)-6-prop-2-ynyloxy-quinazoline-2-carbonyl]-amino}-4-methyl-benzoic acid methyl ester

3-{[4-(4-Isopropyl-phenyl)-6-prop-2-ynyloxy-quinazoline-2-carbonyl]-amino}-4-methoxy-benzoic acid methyl ester

5-{[4-(4-Isopropyl-phenyl)-6-prop-2-ynyloxy-quinazoline-2-carbonyl]-amino}-isophthalic acid dimethyl ester

4-{[4-(4-Isopropyl-phenyl)-6-prop-2-ynyloxy-quinazoline-2-carbonyl]-amino}-phthalic acid dimethyl ester

4-(4-Isopropyl-phenyl)-6-prop-2-ynyloxy-quinazoline-2-carboxylic acid (3,5-dichloro-phenyl)-amide

4-(4-Isopropyl-phenyl)-6-prop-2-ynyloxy-quinazoline-2-carboxylic acid (3,4-dichloro-phenyl)-amide

4-(4-Isopropyl-phenyl)-6-prop-2-ynyloxy-quinazoline-2-carboxylic acid (3-chloro-4-fluoro-phenyl)-amide

4-(4-Isopropyl-phenyl)-6-prop-2-ynyloxy-quinazoline-2-carboxylic acid (4-chloro-3-trifluoromethyl-phenyl)-amide

5-{[4-(4-Isopropyl-phenyl)-6-prop-2-ynyloxy-quinazoline-2-carbonyl]-amino}-pyridine-2-carboxylic acid methyl ester

5-{[4-(4-Isopropyl-phenyl)-6-prop-2-ynyloxy-quinazoline-2-carbonyl]-amino}-nicotinic acid methyl ester

5-{[4-(4-Isopropyl-phenyl)-6-prop-2-ynyloxy-quinazoline-2-carbonyl]-amino}-nicotinic acid isopropyl ester

[4-(4-Isopropyl-phenyl)-6-prop-2-ynyloxy-quinazolin-2-yl]-pyrrol-1-yl-methanone

4-(4-Isopropyl-phenyl)-6-prop-2-ynyloxy-quinazoline-2-carboxylic acid (5-methyl-1H-pyrazol-3-yl)-amide

(2-{[4-(4-Isopropyl-phenyl)-6-prop-2-ynyloxy-quinazoline-2-carbonyl]-amino}-thiazol-4-yl)-acetic acid ethyl ester

4-(4-Isopropyl-phenyl)-6-prop-2-ynyloxy-quinazoline-2-carboxylic acid naphthalen-1-ylamide

4-(4-Isopropyl-phenyl)-6-prop-2-ynyloxy-quinazoline-2-carboxylic acid isoquinolin-8-ylamide

4-(4-Isopropyl-phenyl)-6-prop-2-ynyloxy-quinazoline-2-carboxylic acid phthalazin-5-ylamide

4-(4-Isopropyl-phenyl)-6-prop-2-ynyloxy-quinazoline-2-carboxylic acid quinolin-5-ylamide

4-(4-Isopropyl-phenyl)-6-prop-2-ynyloxy-quinazoline-2-carboxylic acid quinolin-8-ylamide

4-(4-Isopropyl-phenyl)-6-prop-2-ynyloxy-quinazoline-2-carboxylic acid isoquinolin-4-ylamide

4-(4-Isopropyl-phenyl)-6-prop-2-ynyloxy-quinazoline-2-carboxylic acid (5-acetyl-quinolin-8-yl)-amide

4-(4-Isopropyl-phenyl)-6-prop-2-ynyloxy-quinazoline-2-carboxylic acid (3-bromo-6-methoxy-quinolin-8-yl)-amide

4-(4-Isopropyl-phenyl)-6-prop-2-ynyloxy-quinazoline-2-carboxylic acid quinolin-2-ylamide

4-(4-Isopropyl-phenyl)-6-prop-2-ynyloxy-quinazoline-2-carboxylic acid quinolin-6-ylamide

4-(4-Isopropyl-phenyl)-6-prop-2-ynyloxy-quinazoline-2-carboxylic acid (2-methyl-quinolin-6-yl)-amide

(6-{[4-(4-Isopropyl-phenyl)-6-prop-2-ynyloxy-quinazoline-2-carbonyl]-amino}-quinolin-8-yloxy)-acetic acid ethyl ester

4-(4-Isopropyl-phenyl)-6-prop-2-ynyloxy-quinazoline-2-carboxylic acid (1H-benzoimidazol-4-yl)-amide

4-(4-Isopropyl-phenyl)-6-prop-2-ynyloxy-quinazoline-2-carboxylic acid benzothiazol-2-ylamide

4-(4-Isopropyl-phenyl)-6-prop-2-ynyloxy-quinazoline-2-carboxylic acid (benzo[1,3]dioxol-5-ylmethyl)-amide

4-(4-Isopropyl-phenyl)-6-prop-2-ynyloxy-quinazoline-2-carboxylic acid (thiophen-2-ylmethyl)-amide

4-(4-Isopropyl-phenyl)-6-prop-2-ynyloxy-quinazoline-2-carboxylic acid 3-methoxy-phenyl ester

1-(4-Isopropyl-phenyl)-7-prop-2-ynyloxy-isoquinoline-3-carboxylic acid ethyl ester

1-(4-Isopropyl-phenyl)-7-prop-2-ynyloxy-isoquinoline-3-carboxylic acid 1,2-dimethyl-propyl ester

1-(4-Isopropyl-phenyl)-7-prop-2-ynyloxy-isoquinoline-3-carboxylic acid isobutyl ester

1-(4-Isopropyl-phenyl)-7-prop-2-ynyloxy-isoquinoline-3-carboxylic acid cyclopropylmethyl ester

1-(4-Isopropyl-phenyl)-7-prop-2-ynyloxy-isoquinoline-3-carboxylic acid benzyl ester

1-(4-Isopropyl-phenyl)-7-prop-2-ynyloxy-isoquinoline-3-carboxylic acid 2-methoxy-benzyl ester

1-(4-Isopropyl-phenyl)-7-prop-2-ynyloxy-isoquinoline-3-carboxylic acid 3-methoxy-benzyl ester

1-(4-Isopropyl-phenyl)-7-prop-2-ynyloxy-isoquinoline-3-carboxylic acid 4-methoxycarbonyl-benzyl ester

1-(4-Isopropyl-phenyl)-7-prop-2-ynyloxy-isoquinoline-3-carboxylic acid phenethyl ester

1-(4-Isopropyl-phenyl)-7-prop-2-ynyloxy-isoquinoline-3-carboxylic acid 1-phenyl-ethyl ester

1-(4-Isopropyl-phenyl)-7-prop-2-ynyloxy-isoquinoline-3-carboxylic acid [3-(2-hydroxy-ethanesulfonyl)-phenyl]-amide

[1-(4-Isopropyl-phenyl)-7-prop-2-ynyloxy-isoquinolin-3-yl]-(3-methoxy-phenyl)-methanone

[1-(4-Isopropyl-phenyl)-7-prop-2-ynyloxy-isoquinolin-3-yl]-(4-methoxy-phenyl)-methanone.

According to a fifth aspect of the invention there is provided a pharmaceutical composition comprising a compound of formula (I) in association with a pharmaceutically acceptable excipient, diluent or carrier.

According to a sixth aspect of the invention there is provided a compound of formula (I) for promoting the release of parathyroid hormone.

It is now well established that controlled treatment of patients with parathyroid hormone (PTH) and analogues and fragments thereof can have a pronounced anabolic effect on bone formation. Thus compounds which promote PTH release, such as the compounds of the present invention may be used for preventing or treating conditions of bone which are associated with increased calcium depletion or resorption or in which stimulation of bone formation and calcium fixation in the bone is desirable.

Thus in a seventh aspect the invention includes a method for preventing or treating bone conditions which are associated with increased calcium depletion or resorption or in which stimulation of bone formation and calcium fixation in the bone is desirable in which an effective amount of a compound of formula (I) as defined above, or a pharmaceutically-acceptable and -cleavable ester, or acid addition salt thereof is administered to a patient in need of such treatment.

In an eighth aspect the invention provides a process for preparation of a compound of formula (I) in free or salt form, comprising the step of:

(i) for cases where Q is N, reacting a compound of formula (III) with a compound of formula (IV) and an ammonium salt in the presence of a suitable solvent:

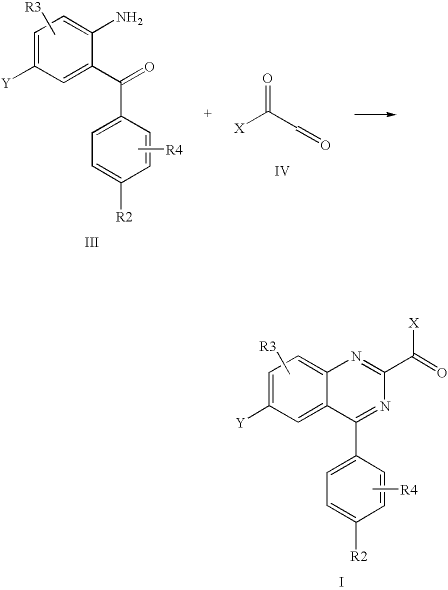

A preferred ammonium salt is ammonium acetate. Preferably the solvent contains water. Suitable solvents are ethanol/water. The presence of an oxidizing agent, e.g. DDQ is also preferred.

The compound of formula III may be prepared by any suitable route, for example, when Y is propargyloxy and R2 is isopropyl, as follows:

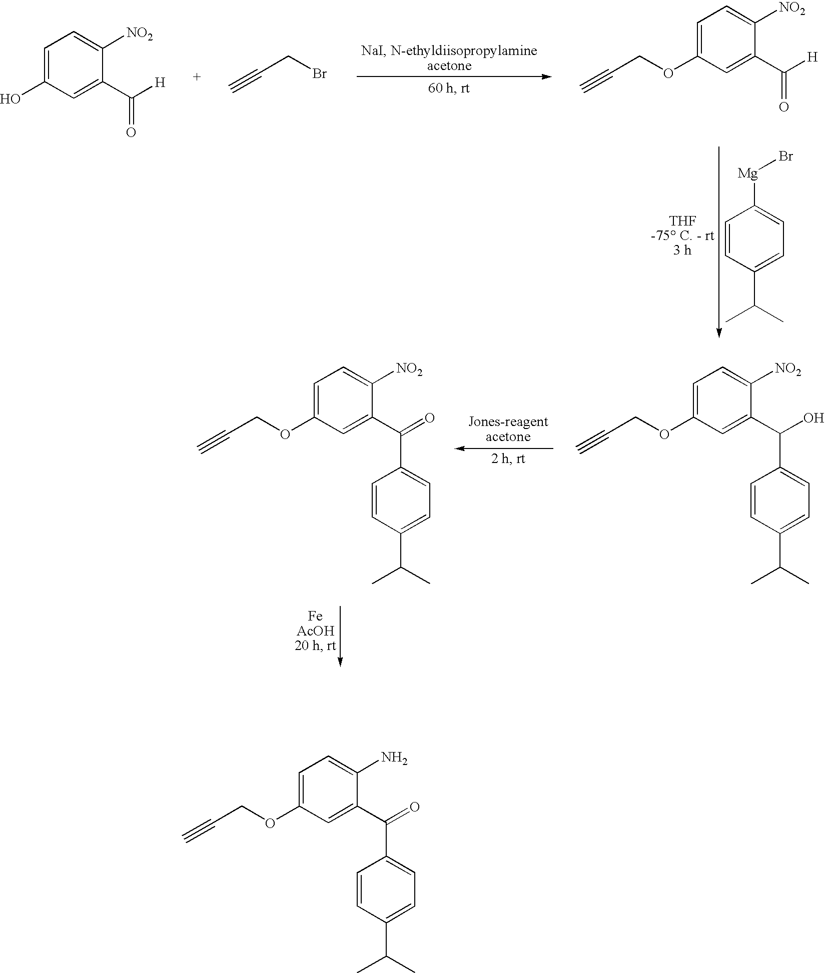

(ii) when Q is CH, reacting a compound of formula V

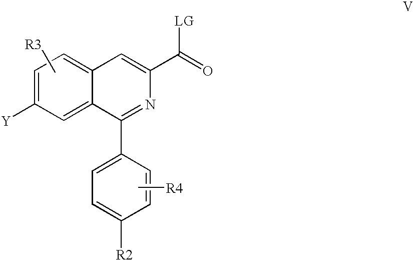

wherein LG represents a suitable leaving group, for example a Weinreb amide (N-methoxy-N-methylamide)

with an organometallic reagent of formula VI:

X-Met VI

where Met is Li, a Grignard reagent (—MgBr) or other suitable organometallic under suitable anhydrous conditions; or

(iii) reacting a compound of formula Va

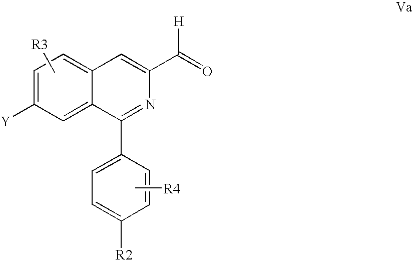

with an organometallic reagent of formula VI:

X-Met VI

under suitable anhydrous conditions followed by oxidation to the carbonyl compound by an appropriate oxidation agent; or

(iv) reacting a compound of formula VII

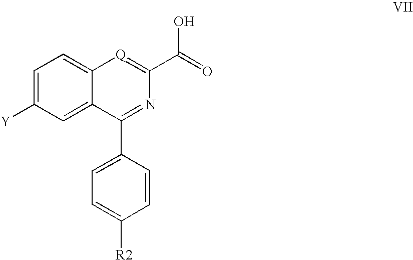

with a compound X—H wherein the H forms part of an amino or hydroxy group, the reaction being carried out in the presence of a coupling reagent; or

(v) reacting a compound of formula VIII

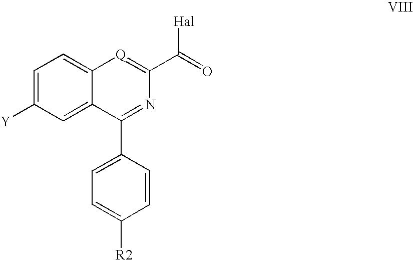

wherein Hal is halogen or a leaving group

with a compound X—H wherein the H forms part of an amino or hydroxy group, the reaction being carried out in the presence of a coupling reagent.

The compound of formula V can be prepared by any suitable route, for example as follows:

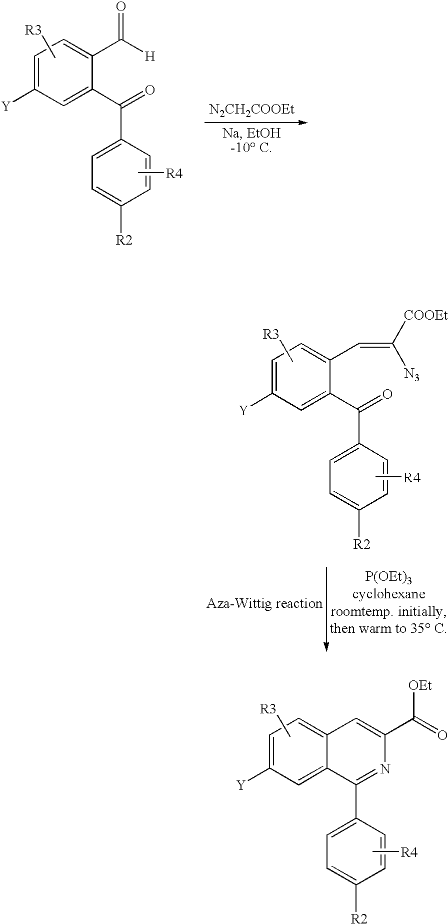

The compounds of formula I in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

In a ninth aspect the invention includes the use of a compound of formula (I) in the manufacture of a medicament for preventing or treating bone conditions which are associated with increased calcium depletion or resorption or in which stimulation of bone formation and calcium fixation in the bone is desirable.

In a tenth aspect the invention provides a combination comprising a therapeutically effective amount of a compound as described above and a second drug substance selected from: calcium, a calcitonin or an analogue or derivative thereof, a steroid hormone, a partial estrogen agonist or estrogen-gestagen combination, a SERM (Selective Estrogen Receptor Modulator), vitamin D or an analogue thereof or PTH, a PTH fragment or a PTH derivative for simultaneous, separate or sequential treatment.

Agents of the invention may be prepared by processes described below, which are intended to be non-limiting examples:

The analytical HPLC conditions are as follows:

| | |
|---|---|
| Instrument | Agilent 110 System with G1311A quarternary pump |
| and settings: | (0.8 ml dead volume), G1313A autosampler |
| | (1 μl injection volume), G1316A column |
| | compartment (35° C.), G1315A diode array |
| | detector (detection by UV absorption at 210 nm-250 |
| | nm wave length), G1946A mass spectrometer with |
| | APC ionization. |
| Column: | Waters Symmetry C8, 50 × 2.1 mm, 3.5 μm |
| | mean particle size. flow rate 1.0 ml/min. |
| Linear gradient: | 5% B in A to 95% B in A within 2.0 min. |
| | A: water containing 5% acetonitirile and 0.1% TFA; |
| | B: acetonitrile containing 0.1% TFA. |

### EXAMPLE 1

### (4-tert-Butyl-phenyl)-[4-(4-isopropyl-phenyl)-6-propargyloxy-quinazolin-2-yl]-methanone

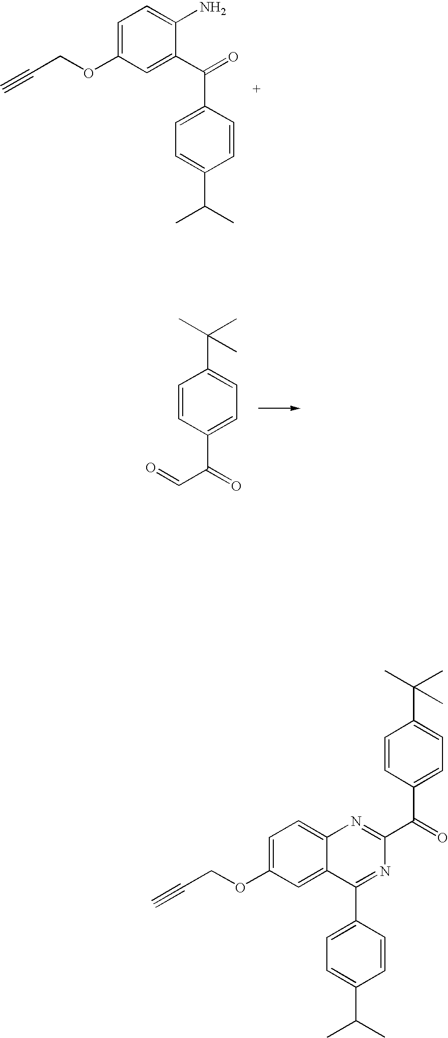

A mixture of 250 mg (0.85 mmol) (2-amino-5-propargyloxy-phenyl)-(4-isopropyl-phenyl)-methanone and 20 mg (2.6 mmol) ammonium acetate is dissolved in 2.5 ml ethanol and 0.82 ml water. To this mixture, 243 mg (1.28 mmol) of (4-tert-butyl-phenyl)-oxo-acetaldehyd is added and stirring continued for 8 hours at rt. After extraction with water/diethyl ether the organic layer is dried over magnesium sulfate and concentrated under reduced pressure to yield a yellow oil. This is purified by chromatography (hexane/ethyl acetate). After concentration and drying under HV the product is treated with a mixture of diethyl ether/petroleum ether to yield a yellow solid.

m.p. 166-168° C.

¹H-NMR (300 MHz, CDCl₃): 8.21 (d, 1H), 8.13 (d, 2H), 7.85 (d, 2H), 7.66-7.72 (m, 2H), 7.52 (d, 2H), 7.44 (d, 2H), 4.82 (d, 2H), 3.05 (kept, 1H), 2.64 (t, 1H), 1.38 (s, 9H), 1.35 (d, 6H).

MS: 463 (M+1)⁺

Preparation of the starting material:

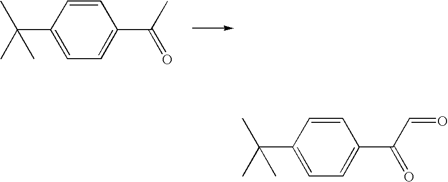

A mixture of 1.26 g (11.3 mmol) selenium dioxide in 11 ml of a dioxane/water (30:1) is warmed to 50° C. On obtaining a clear solution 2.0 g (11.3 mmol) of 4-tert-butyl-acetophenone is added in portions and the resulting mixture is stirred overnight at reflux. The solid parts of the resulting suspension are separated off and the solution is concentrated in vacuo. The residue is distributed between ethyl acetate and water, the organic layer dried over magnesium sulfate and concentrated. Purification by chromatography (hexane/ethyl acetate) yields (4-tert-butyl-phenyl)-oxo-acetaldehyde as a yellow oil which solidifies after drying under HV.

The compounds of the following examples are prepared in an analogous manner using the appropriate starting materials:

### EXAMPLE 2

### [4-(4-Isopropyl-phenyl)-6-propargyloxy-quinazolin-2-yl]-phenyl-methanone

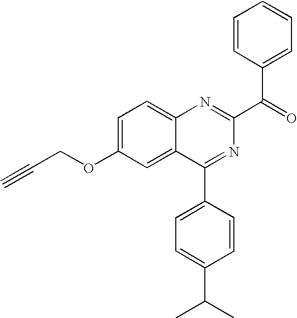

¹H-NMR (CDCl₃, 300 MHz): 8.15 (d, 1H), 8.12 (d, 2H), 7.80 (d, 2H), 7.65-7.60 (m, 2H), 7.56 (t, 1H), 7.44 (t, 2H), 7.38 (d, 2H), 4.76 (d, 2H), 2.99 (hept, 1H), 2.59 (t, 1H), 1.29 (d, 6H).

MS: 407 (M+1)⁺

The starting material phenylglyoxal monohydrate is commercially available.

### EXAMPLE 3

### (2-Methoxy-phenyl)-[4-(4-Isopropyl-phenyl)-6-propargyloxy-quinazolin-2-yl]-methanone

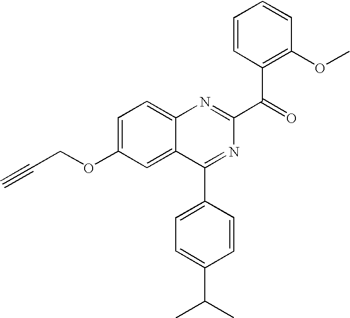

¹H-NMR (CDCl₃, 300 MHz): 8.17 (d, 1H), 7.83 (dd, 1H), 7.77 (d, 2H), 7.66-7.61 (m, 2H), 7.52 (td, 1H), 7.39 (d, 2H), 7.09 (td, 1H), 6.95 (d, 1H), 4.79 (d, 2H), 3.53 (s, 3H), 3.01 (hept, 1H), 2.63 (t, 1H), 1.32 (d, 6H).

MS: 437 (M+1)⁺

The starting material 2-methoxyphenylglyoxal hydrate is commercially available.

### EXAMPLE 4

### (3-Methoxy-phenyl)-[4-(4-Isopropyl-phenyl)-6-propargyloxy-quinazolin-2-yl]-methanone

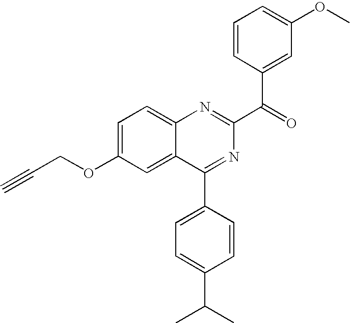

¹H-NMR (CDCl₃, 300 MHz): 8.23 (d, 1H), 7.85 (d, 2H), 7.75-7.66 (m, 4H), 7.44 (d, 2H), 7.38 (t, 1H), 7.17 (ddd, 1H), 4.82 (d, 2H), 3.88 (s, 3H), 3.04 (hept, 1H), 2.64 (t, 1H), 1.34 (d, 6H).

MS: 437 (M+1)⁺

The starting material 3-methoxyphenylglyoxal hydrate is commercially available.

### EXAMPLE 5

### (4-Methoxy-phenyl)-[4-(4-Isopropyl-phenyl)-6-propargyloxy-quinazolin-2-yl]-methanone

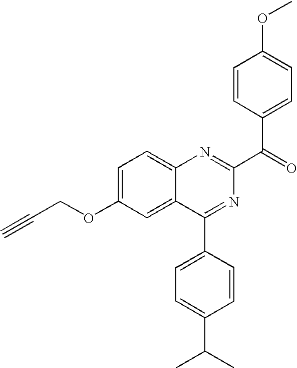

¹H-NMR (CDCl₃, 300 MHz): 8.23 (dd, 1H), 8.18 (d, 2H),7.84 (d, 2H), 7.70-7.65 (m, 2H), 7.43 (d, 2H), 6.97 (d, 2H), 4.81 (d, 2H), 3.90 (s, 3H), 3.03 (hept, 1H), 2.63 (t, 1H), 1.34 (d, 6H).

MS: 437 (M+1)⁺

The starting material 4-methoxyphenylglyoxal hydrate is prepared according to the literature, for example by SeO₂ oxidation of (4-methoxy-phenyl)-ethanone.

### EXAMPLE 6

### (4-Fluoro-phenyl)-[4-(4-isopropyl-phenyl)-6-propargyloxy-quinazolin-2-yl]-methanone

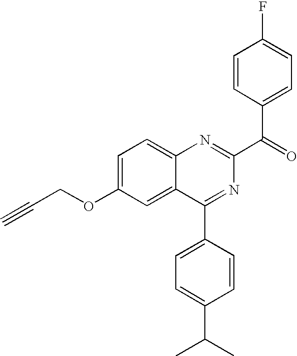

m. p. 124-126° C.

¹H-NMR (300 MHz, CDCl₃): 8.15-8.28 (m, 3H), 7.83 (d, 21-1), 7.65-7.70 (m, 2H), 7.43 (d, 2H), 7.12-7.20 (m, 2H), 4.80 (d, 2H), 3.04 (hept, 1H), 2.63 (t, 1H), 1.33 (d, 6H).

MS: 425 (M+1)⁺

The starting material (4-fluoro-phenyl)-oxo-acetaldehyde is prepared according to the literature, for example by SeO₂ oxidation of 1-(4-fluoro-phenyl)-ethanone, analogously to Example 1.

### EXAMPLE 7

### (3-Fluoro-phenyl)-[4-(4-isopropyl-phenyl)-6-propargyloxy-quinazolin-2-yl]-methanone

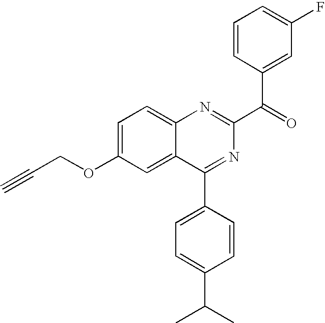

m. p. 118-120° C.

¹H-NMR (300 MHz, CDCl₃): 8.19 (d, 1H), 7.97 (d, 1H), 7.91 (d, 1H), 7.83 (d, 2H), 7.65-7.72 (m, 2H), 7.40-7.51 (m, 3H), 7.31 (td, 1H), 4.81 (d, 2H), 3.04 (hept, 1H), 2.63 (t, 1H), 1.34 (d, 6H).

MS: 425 (M+1)⁺

The starting material (3-fluoro-phenyl)-oxo-acetaldehyde is prepared according to the literature, for example by SeO₂ oxidation of 1-(3-fluoro-phenyl)-ethanone, analogously to Example 1.

### EXAMPLE 8

### (3-Chloro-phenyl)-[4-(4-isopropyl-phenyl)-6-propargyloxy-quinazolin-2-yl]-methanone

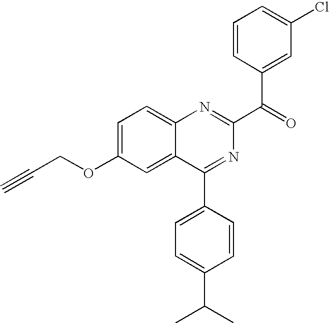

m. p. 128-130° C.

¹H-NMR (300 MHz, CDCl₃): 8.16-8.22 (m, 2H), 8.07 (d, 1H), 7.83 (d, 2H), 7.65-7.72 (m, 2H), 7.68 (d, 1H), 7.40-7.48 (m, 3H), 4.81 (d, 2H), 3.04 (hept, 1H), 2.63 (t, 1H), 1.34 (d, 6H).

MS: 441 (M+1)⁺

The starting material (3-chloro-phenyl)-oxo-acetaldehyde is prepared according to the literature, for example by SeO₂ oxidation of (3-chloro-phenyl)-ethanone, analogously to Example 1.

### EXAMPLE 9

### (4-Chloro-phenyl)-[4-(4-isopropyl-phenyl)-6-propargyloxy-quinazolin-2-yl]-methanone

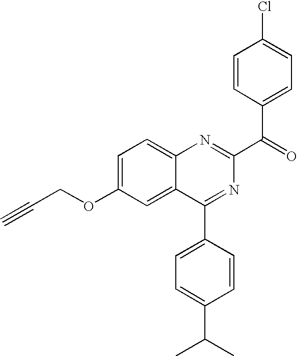

m. p. 108-110° C.

¹H-NMR (300 MHz, CDCl₃): 8.12-8.20 (m, 3H), 7.83 (d, 2H), 7.65-7.72 (m, 2H), 7.40-7.50 (m, 4H), 4.81 (d, 2H), 3.04 (hept, 1H), 2.63 (t, 1H), 1.34 (d, 6H).

MS: 441 (M+1)⁺

The starting material (4-chloro-phenyl)-oxo-acetaldehyde is prepared according to the literature, for example by SeO₂ oxidation of (4-chloro-phenyl)-ethanone, analogously to Example 1.

### EXAMPLE 10

### (4-Fluoro-phenyl)-[4-(4-tert.butyl-phenyl)-6-propargyloxy-quinazolin-2-yl]-methanone

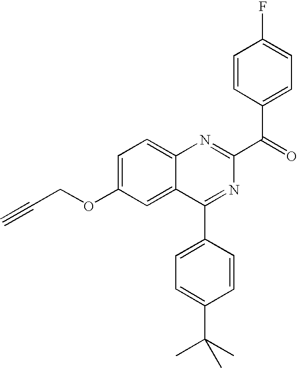

m. p. 105-107° C.

¹H-NMR (300 MHz, CDCl₃): 8.15-8.27 (m, 3H), 7.84 (d, 2H), 7.65-7.72 (m, 2H), 7.59 (d, 2H), 7.17 (t, 2H), 4.81 (d, 2H), 2.63 (t, 1H), 1.41 (s, 9H).

MS: 439 (M+1)⁺

The starting material (4-fluoro-phenyl)-oxo-acetaldehyde is prepared according to the literature, for example by SeO₂ oxidation of (4-fluoro-phenyl)-ethanone, analogously to Example 1.

### EXAMPLE 11

### (3-Fluoro-phenyl)-[4-(4-tert.butyl-phenyl)-6-propargyloxy-quinazolin-2-yl]-methanone

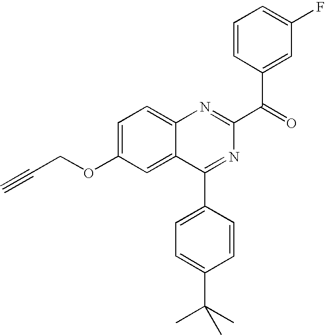

m. p. 156-158° C.

¹H-NMR (300 MHz, CDCl₃): 8.20 (d, 1H), 7.97 (d, 1H), 7.91 (d, 1H), 7.84 (d, 2H), 7.65-7.73 (m, 2H), 7.59 (d, 2H), 7.33-7.52 (m 1H), 7.31 (td, 1H), 4.81 (d, 2H), 2.63 (t, 1H), 1.41 (s, 9H).

MS: 439 (M+1)⁺

The starting material (3-fluoro-phenyl)-oxo-acetaldehyde is prepared according to the literature, for example by SeO₂ oxidation of (3-fluoro-phenyl)-ethanone, analogously to Example 1.

### EXAMPLE 12

### (3-Bromo-phenyl)-[4-(4-isopropyl-phenyl)-6-propargyloxy-quinazolin-2-yl]-methanone

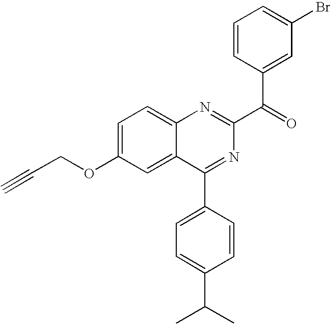

m. p. 132-132° C.

¹H-NMR (300 MHz, CDCl₃): 8.34 (br t, 1H), 8.19 (d, 1H), 8.10 (d, 1H), 7.83 (d, 2H), 7.65-7.76 (m, 3H), 7.43 (d, 2H), 7.37 (t, 1H), 4.81 (d, 2H), 3.03 (hept, 1H), 2.63 (t, 1H), 1.33 (d, 6H).

MS: 485/487 (M+1)⁺

The starting material (3-bromo-phenyl)-oxo-acetaldehyde is prepared according to the literature, for example by SeO₂ oxidation of (3-bromo-phenyl)-ethanone, analogously to Example 1.

### EXAMPLE 13

### (4-Bromo-phenyl)-[4-(4-isopropyl-phenyl)-6-propargyloxy-quinazolin-2-yl]-methanone

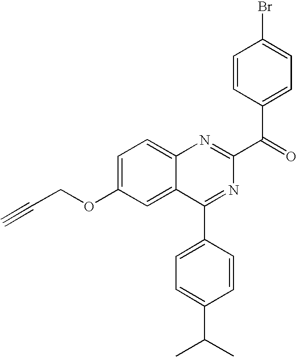

m. p. 97-100° C.

¹H-NMR (300 MHz, CDCl₃): 8.18 (d, 1H), 8.07 (d, 2H), 7.82 (d, 2H), 7.61-7.72 (m, 4H), 7.43 (m, 2H), 4.81 (d, 2H), 3.04 (hept, 1H), 2.63 (t, 1H), 1.34 (d, 6H).

MS: 485/487 (M+1)⁺

The starting material (4-bromo-phenyl)-oxo-acetaldehyde is prepared according to the literature, for example by SeO₂ oxidation of (4-bromo-phenyl)-ethanone, analogously to Example 1.

### EXAMPLE 14

### (4-Methyl-phenyl)-[4-(4-isopropyl-phenyl)-6-propargyloxy-quinazolin-2-yl]-methanone

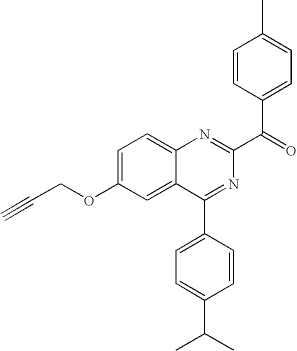

m. p. 130-132° C.

¹H-NMR (300 MHz, CDCl₃): 8.18 (d, 1H), 8.06 (d, 2H), 7.83 (d, 2H), 7.63-7.70 (m, 2H), 7.12 (d, 2H), 7.29 (d, 2H), 4.80 (d, 2H), 3.03 (hept, 1H), 2.60 (t, 1H), 2.44 (s, 3H), 1.33 (d, 6H).

MS: 421 (M+1)⁺

The starting material (4-methyl-phenyl)-oxo-acetaldehyde is prepared according to the literature, for example by SeO₂ oxidation of (4-methyl-phenyl)-ethanone, analogously to Example 1.

### EXAMPLE 15

### (4-Isopropyl-phenyl)-[4-(4-isopropyl-phenyl)-6-propargyloxy-quinazolin-2-yl]-methanone

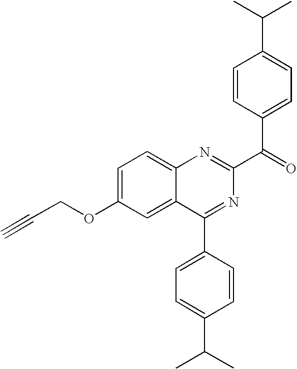

m. p. 132-134° C.

¹H-NMR (300 MHz, CDCl₃): 8.18 (d, 1H), 8.09 (d, 2H), 7.83 (d, 2H), 7.63-7.70 (m, 2H), 7.42 (d, 2H), 7.34 (d, 2H), 4.80 (d, 2H), 3.03 (hept, 1H), 2.99 (hept, 1H), 2.62 (t, 1H), 1.32 (d, 6H), 1.30 (d, 6H).

MS: 449 (M+1)⁺

The starting material (4-isopropyl-phenyl)-oxo-acetaldehyde is prepared according to the literature, for example by SeO₂ oxidation of (4-isopropyl-phenyl)-ethanone, analogously to Example 1.

### EXAMPLE 16

### (4-Ethyl-phenyl)-[4-(4-isopropyl-phenyl)-6-propargyloxy-quinazolin-2-yl]-methanone

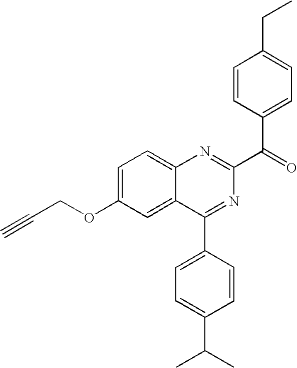

m. p. 108-111° C.

¹H-NMR (300 MHz, CDCl₃): 8.16 (d, 1H), 8.08 (d, 2H), 7.83 (d, 2H), 7.63-7.70 (m, 2H), 7.42 (d, 2H), 7.31 (d, 2H), 4.80 (d, 2H), 3.03 (hept, 1H), 2.74 (q, 2H), 2.62 (t, 1H), 1.33 (d, 6H), 1.28 (t, 3H).

MS: 435 (M+1)⁺

The starting material (4-ethyl-phenyl)-oxo-acetaldehyde is prepared according to the literature, for example by SeO₂ oxidation of (4-ethyl-phenyl)-ethanone, analogously to Example 1.

### EXAMPLE 16a

### (4-Propyl-phenyl)-[4-(4-isopropyl-phenyl)-6-propargyloxy-quinazolin-2-yl]-methanone

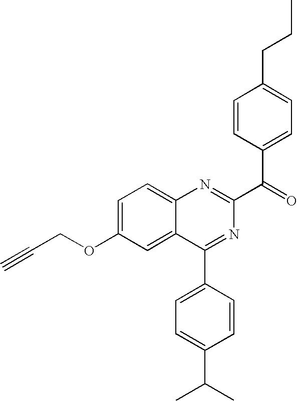

m. p. 140-142° C.

¹H-NMR (300 MHz, CDCl₃): 8.18 (d, 1H), 8.05 (d, 2H), 7.83 (d, 2H), 7.62-7.70 (m, 2H), 7.42 (d, 2H), 7.28 (d, 2H), 4.80 (d, 2H), 3.03 (hept, 1H), 2.67 (t, 2H), 2.63 (t, 1H), 1.68 (m, 2H) 1.32 (d, 6H), 0.97 (t, 3H).

MS: 449 (M+1)⁺

The starting material (4-n-propyl-phenyl)-oxo-acetaldehyde is prepared according to the literature, for example by SeO₂ oxidation of (4-n-propyl-phenyl)-ethanone, analogously to Example 1.

### EXAMPLE 17

### (4-Cyano-phenyl)-[4-(4-isopropyl-phenyl)-6-propargyloxy-quinazolin-2-yl]-methanone

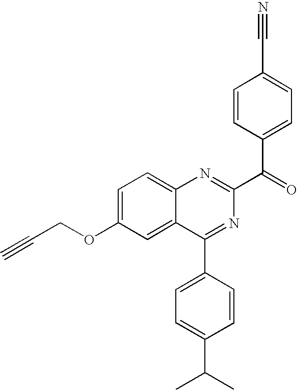

m. p. 130-132° C.

¹H-NMR (300 MHz, CDCl₃): 8.31 (d, 2H), 8.20 (d, 1H), 7.82 (t, 4H), 7.64-7.75 (m, 2H), 7.45 (d, 2H), 4.82 (d, 2H), 3.05 (hept, 1H), 2.62 (broad, 1H), 1.35 (d, 6H).

MS: 432 (M+1)⁺

The starting material (4-cyano-phenyl)-oxo-acetaldehyde is prepared according to the literature, for example by SeO₂ oxidation of (4-cyano-phenyl)-ethanone, analogously to Example 1.

### EXAMPLE 18

### (4-Methylthio-phenyl)-[4-(4-isopropyl-phenyl)-6-propargyloxy-quinazolin-2-yl]-methanone

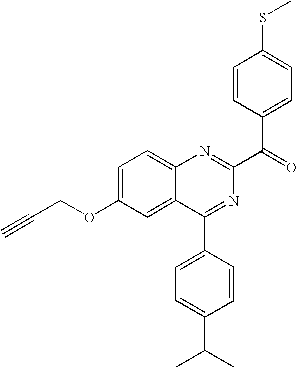

m. p. 161-164° C.

¹H-NMR (300 MHz, CDCl₃): 8.19 (d, 1H), 8.11 (d, 2H), 7.84 (d, 2H), 7.64-7.72 (m, 2H), 7.43 (d, 2H), 7.31 (d, 2H), 4.81 (d, 2H), 3.04 (hept, 1H), 2.63 (t, 1H), 2.55 (s, 3H), 1.34 (d, 6H).

MS: 453 (M+1)⁺

The starting material (4-methylthiophenyl)-oxo-acetaldehyde is prepared according to the literature, for example by SeO₂ oxidation of (4-methylthiophenyl)-ethanone, analogously to Example 1.

### EXAMPLE 19

### (4-Methansulfonyl-phenyl)-[4-(4-isopropyl-phenyl)-6-propargyloxy-quinazolin-2-yl]-methanone

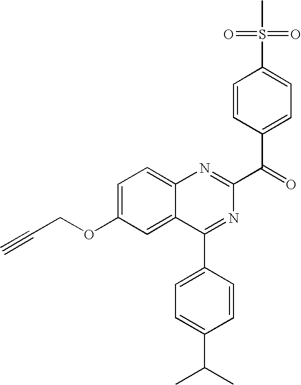

m. p. 181-184° C.

¹H-NMR (300 MHz, CDCl₃): 8.40 (d, 2H), 8.21 (d, 1H), 8.10 (d, 2H), 7.84 (d, 2H), 7.70-7.75 (m, 2H), 7.46 (d, 2H), 4.83 (d, 2H), 3.13 (s, 3H), 3.06 (hept, 1H), 2.65 (broad, 1H), 1.36 (d, 6H).

MS: 485 (M+1)⁺

The starting material (4-methanesulfonyl-phenyl)-oxo-acetaldehyde is prepared according to the literature, for example by SeO₂ oxidation of (4-methanesulfonyl-phenyl)ethanone, analogously to Example 1.

### EXAMPLE 20

### (4-Dimethylamino-phenyl)-[4-(4-isopropyl-phenyl)-6-propargyloxy-quinazolin-2-yl]-methanone

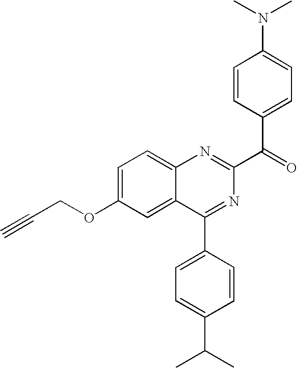

m. p. 148-151° C.

¹H-NMR (300 MHz, CDCl₃): 8.18 (d, 1H), 8.08 (d, 2H), 7.85 (d, 2H), 7.60-7.70 (m, 2H), 7.42 (d, 2H), 6.68 (d, 2H), 4.80 (d, 2H), 3.09 (s, 6H), 3.03 (hept, 1H), 2.63 (t, 1H), 1.34 (d, 6H).

MS: 450 (M+1)⁺

The starting material (4-dimethylamino-phenyl)-oxo-acetaldehyde is prepared according to the literature, for example by SeO₂ oxidation of (4-dimethylamino-phenyl)-ethanone, analogously to Example 1.

### EXAMPLE 21

### (4-Ethoxy-phenyl)-[4-(4-isopropyl-phenyl)-6-propargyloxy-quinazolin-2-yl]-methanone

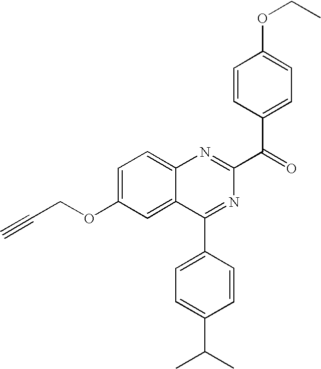

m. p. 117-119° C.

¹H-NMR (300 MHz, CDCl₃): 8.15-8.22 (m, 3H), 7.85 (d, 2H), 7.65-7.73 (m, 2H), 7.45 (d, 2H), 6.97 (d, 2H), 4.82 (br s, 2H), 4.11 (q, 2H), 3.06 (hept, 1H), 2.65 (broad, 1H), 1.48 (t, 3H), 1.36 (d, 6H).

MS: 451 (M+1)⁺

The starting material (4-ethoxy-phenyl)-oxo-acetaldehyde is prepared according to the literature, for example by SeO₂ oxidation of (4-ethoxy-phenyl)-ethanone, analogously to Example 1.

### EXAMPLE 22

### 4-[4-(4-Isopropyl-phenyl)-6-propargyloxy-quinazoline-2-carbonyl]-benzoic acid methyl ester

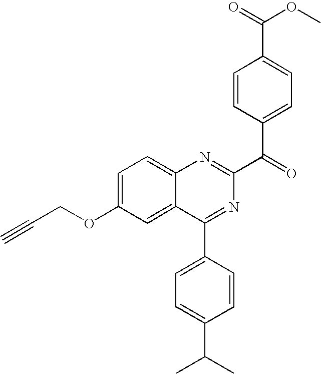

m. p. 108-110° C.

¹H-NMR (300 MHz, CDCl₃): 8.12-8.25 (m, 5H), 7.82 (d, 2H), 7.65-7.72 (m, 2H), 7.42 (d, 2H), 4.81 (d, 2H), 3.96 (s, 3H), 3.03 (kept, 1H), 2.63 (t, 1H), 1.33 (d, 6H).

MS: 465 (M+1)⁺

The appropriate glyoxal starting material is prepared according to the literature, for example by SeO₂ oxidation of 4-(2-oxo-acetyl)-benzoic acid methyl ester, analogously to Example 1.

### EXAMPLE 23

### (4-Dimethylamino-phenyl)-[4-(4-tert.butyl-phenyl)-6-propargyloxy-quinazolin-2-yl]-methanone

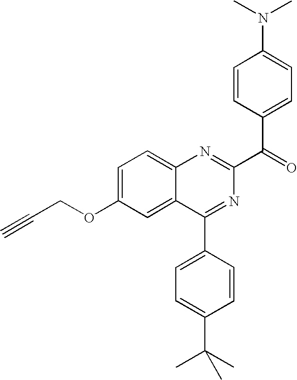

m. p. 150-152° C.

¹H-NMR (300 MHz, CDCl₃): 8.17 (d, 1H), 8.07 (d, 2H), 7.85 (d, 2H), 7.61-7.70 (m, 2H), 7.57 (d, 2H), 6.67 (d, 2H), 4.79 (d, 2H), 3.08 (s, 6H), 2.62 (t, 1H), 1.40 (s, 9H).

MS: 464 (M+1)⁺

The appropriate glyoxal starting material is prepared according to the literature, for example by SeO₂ oxidation of the corresponding ketone, analogously to Example 1.

### EXAMPLE 24

### (4-Dimethylamino-phenyl)-[4-(4-cyclopropyl-phenyl)-6-propargyloxy-quinazolin-2-yl]-methanone

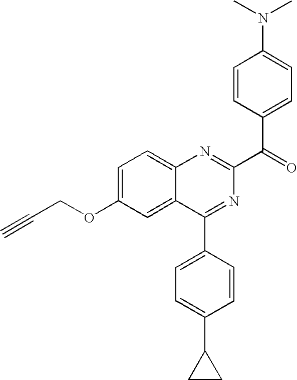

m. p. 169-172° C.

¹H-NMR (300 MHz, CDCl₃): 8.16 (d,1H), 8.07 (d, 2H), 7.80 (d, 2H), 7.60-7.66 (m, 2H), 7.23 (d, 2H), 6.67 (d, 2H), 4.78 (d, 2H), 3.08 (s, 6H), 2.62 (t, 1H), 1.96-206 (m, 1H), 1.04-1.11 (m, 2H), 0.78-0.85 (m, 2H).

MS: 448 (M+1)⁺

### EXAMPLE 25

### 4-[4-(4-Isopropyl-phenyl)-6-propargyloxy-quinazoline-2-carbonyl]-benzoic acid ethyl ester

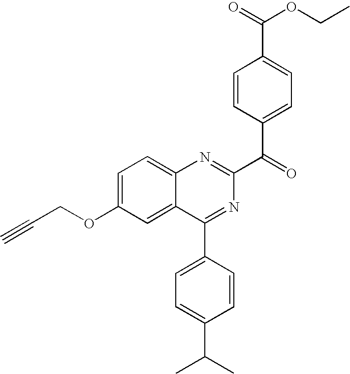

m. p. 132-134° C.

¹H-NMR (300 MHz, CDCl₃): 8.13-8.26 (m, 5H), 7.83 (d, 2H), 7.65-7.72 (m, 2H), 7.41 (d, 2H), 4.81 (d, 2H), 4.42 (q, 2H), 3.04 (hept, 1H), 2.63 (t, 1H), 1.43 (t, 3H), 1.33 (d, 6H).

MS: 479 (M+1)⁺

### EXAMPLE 26

### (4-Methoxy-phenyl)-[4-(4-tert.butyl-phenyl)-6-propargyloxy-quinazolin-2-yl]-methanone

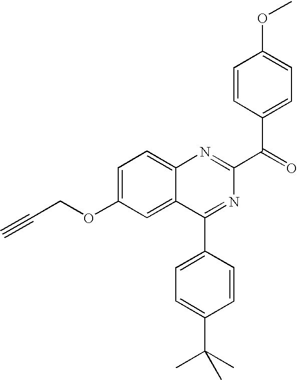

m. p. 150-152° C.

¹H-NMR (300 MHz, CDCl₃): 8.13-8.21 (m, 3H), 7.84 (d, 2H), 7.62-7.71 (m, 2H), 7.57 (d, 2H), 6.96 (d, 2H), 4.80 (d, 2H), 3.89 (s, 3H), 2.62 (broad, 1H), 1.40 (s, 9H).

MS: 451 (M+1)⁺

### EXAMPLE 27

### (4-Ethoxy-phenyl)-[4-(4-cyclopropyl-phenyl)-6-propargyloxy-quinazolin-2-yl]-methanone

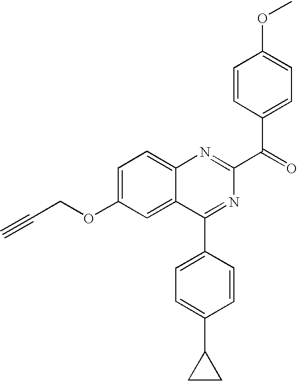

m. p. 147-149° C.

¹H-NMR (300 MHz, CDCl₃): 8.13-8.20 (m, 3H), 7.80 (d, 2H), 7.62-7.68 (m, 2H), 7.24 (d, 2H), 6.96 (d, 2H), 4.78 (d, 2H), 3.89 (s, 3H), 2.62 (broad, 1H), 1.95-2.06 (m, 1H), 1.03-1.13 (m, 2H), 0.77-0.86 (m, 2H).

MS: 435 (M+1)⁺

### EXAMPLE 28

### (3-Ethoxy-4-methoxy-phenyl)-[4-(4-isopropyl-phenyl)-6-propargyloxy-quinazolin-2-yl]-methanone

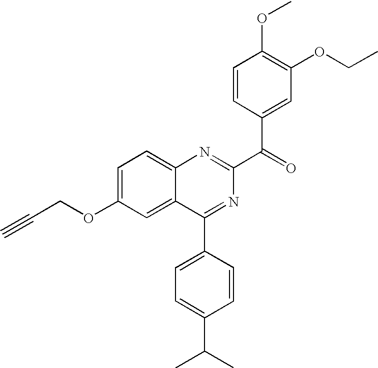

m. p. 148° C.

¹H-NMR (300 MHz, CDCl₃): 8.17 (d, 1H), 7.80-7.86 (m, 3H), 7.63-7.75 (m, 3H), 7.12 (d, 2H), 6.88 (d, 1H), 4.80 (d, 2H), 4.18 (q, 2H), 3.95 (s, 3H), 3.03 (hept, 1H), 2.62 (t, 1H), 1.50 (t, 3H), 1.33 (d, 6H).

MS: 481 (M+1)⁺

Preparation of starting material:

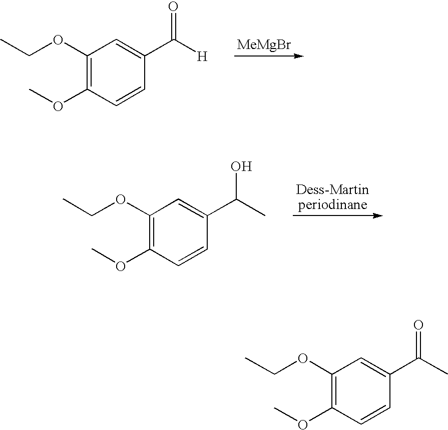

### A) 1-(3-Ethoxy-4-methoxy-phenyl)-ethanol

A solution of 2.0 g (11.1 mmol) 3-ethoxy-4-methoxy-benzaldehyde in 15 ml tetrahydrofurane is slowly treated with 4.4 ml of a etheral 3 M methylmagesiumbromide solution such that the temperature is maintained between −65 and −70° C. After ca. 15 minutes the cooling bath is removed and the mixture allowed to come to room temperature. The resulting mixture is poured into saturated ammonium chloride solution and the alcohol extracted with diethyl ether. The combined organic layers are washed several times with brine, dried over MgSO₄ and concentrated in vacuao. The crude product is directly used for the following oxidation.

¹H-NMR (300 MHz, CDCl₃): 6.94 (d, 1H), 6.88 (dd, 1H), 6.82 (d, 1H), 4.84 (q, 1H), 4.12 (q, 2H), 3.86 (s, 3H), 1.77 (br, OH), 1.48 (d, 3H), 1.47 (t, 3H).

### B) 1-(3-Ethoxy-4-methoxy-phenyl)ethanone

The crude product (1.0 g; 5.10 mmol) obtained in step A is dissolved in 30 ml dichloromethane and treated at room temperature with 2.38 g (5.61 mmol) Dess-Martin periodinane. The oxidation is complete after 4 hours. The white suspension is concentrated i.V. and the product purified by chromatography (hexane/ethyl acetate).

m. p. 71-72° C.

¹H-NMR (300 MHz, CDCl₃): 7.56 (dd, 1H), 7.51 (d, 1H), 6.88 (d, 1H), 4.16 (q, 2H), 3.94 (s, 3H), 2.56 (s, 3H), 1.49 (t, 3H).

The 1-(3-ethoxy-4-methoxy-phenyl)-ethanone thus obtained is oxidized to the corresponding glyoxal as described in example 1.

### EXAMPLE 29

### (4-tert.Butyloxy-phenyl)-[4-(4-isopropyl-phenyl)-6-propargyloxy-quinazolin-2-yl]-methanone

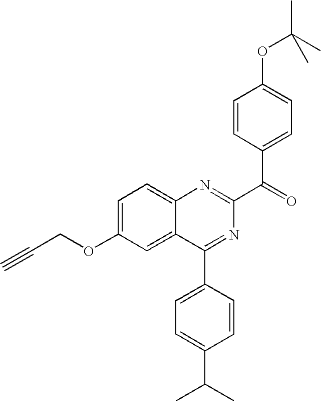

m. p. 130-132° C.

¹H-NMR (300 MHz, CDCl₃): 8.17 (d, 1H), 8.10 (d, 2H), 7.82 (d, 2H), 7.62-7.70 (m, 2H), 7.41 (d, 2H), 7.04 (d, 2H), 4.79 (d, 2H), 3.02 (hept, 1H), 2.62 (t, 1H), 1.49 (s, 9H), 1.32 (d, 6H).

MS: 479 (M+1)³⁰

### Synthesis of 1-(4-tert.butoxy-phenyl)-ethanone as described for example 29.

### EXAMPLE 30

### (4-Hydroxy)-[4-(4-isopropyl-phenyl)-6-propargyloxy-quinazolin-2-yl]-methanone

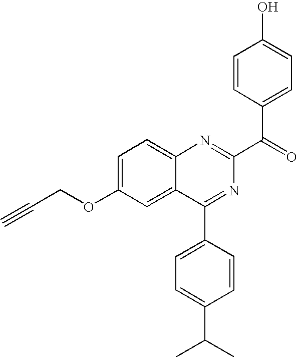

m. p. 185-187° C.

¹H-NMR (300 MHz, CDCl₃): 8.16 (d, 1H), 8.10 (d, 2H), 7.82 (d, 2H), 7.63-7.70 (m, 2H), 7.41 (d, 2H), 6.88 (d, 2H), 5.82 (broad, OH), 4.79 (d, 2H), 3.02 (hept, 1H), 2.62 (t, 1H), 1.32 (d, 6H).

MS: 423 (M+1)⁺

### EXAMPLE 31

### (4-Butyloxy)-[4-(4-isopropyl-phenyl)-6-propargyloxy-quinazolin-2-yl]-methanone

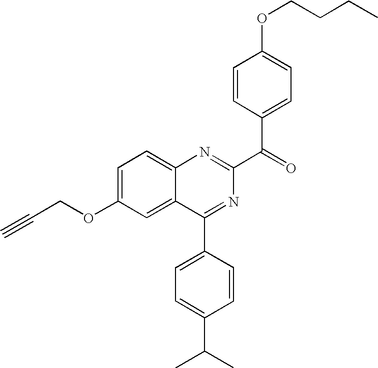

m. p. 91-93° C.

¹H-NMR (300 MHz, CDCl₃): 8.12-8.20 (m, 3H), 7.83 (d, 2H), 7.63-7.70 (m, 2H), 7.42 (d, 2H), 6.94 (d, 2H), 4.81 (d, 2H), 4.05 (t, 2H), 3.03 (hept, 1H), 2.62 (t, 1H), 1.75-1.86 (m, 2H), 1.44-1.55 (m, 2H), 1.33 (d, 6H), 0.99 (t, 3H).

MS: 479 (M+1)⁺

### EXAMPLE 32

### Furan-2-yl-[4-(4-isopropyl-phenyl)-6-propargyloxy-quinazolin-2-yl]-methanone

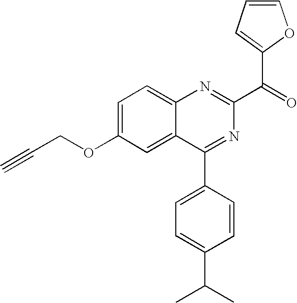

m. p. 150-151° C.

¹H-NMR (300 MHz, CDCl₃): 8.23-8.29 (m, 1H), 8.03 (d, 1H), 7.84 (d, 2H), 7.75-7.78 (m, 1H), 7.64-7.70 (m, 2H), 7.45 (d, 2H), 6.63 (dd, 1H), 4.80 (d, 2H), 3.05 (hept., 1H), 2.62 (t, 1H), 1.35 (d, 6H).

MS: 397 (M+1)⁺

Preparation of furan-2-yl-oxo-acetaldehyde as described in EP 201 221.

### EXAMPLE 33

### Furan-3-yl-[4-(4-tert.butyl-phenyl)-6-propargyloxy-quinazolin-2-yl]-methanone

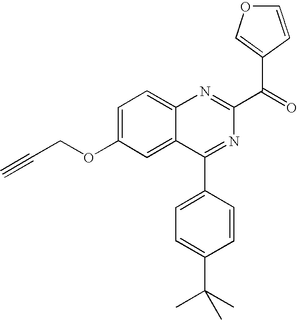

m. p. 170° C.

¹H-NMR (300 MHz, CDCl₃): 8.93-8.95 (m, 1H), 8.24-8.27 (m, 1H), 7.84 (d, 2H), 7.63-7.70 (m, 2H), 7.61 (d, 2H), 7.48 (t, 1H), 7.16 (dd, 1H), 4.81 (d, 2H), 2.62 (t, 1H), 1.43 (s, 9H).

MS: 411 (M+1)⁺

Preparation of 1-furan-3-yl-ethanone as described in EP 230 053, followed by SeO₂ oxidation as described above.

### EXAMPLE 34

### Furan-3-yl-[4-(4-isopropyl-phenyl)-6-propargyloxy-quinazolin-2-yl]-methanone

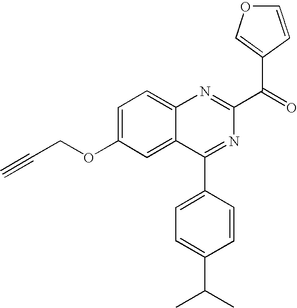

m. p. 133° C.

¹H-NMR (300 MHz, CDCl₃): 8.93-8.95 (m, 1H), 8.24-8.27 (m, 1H), 7.84 (d, 2H), 7.64-7.70 (m, 2H), 7.43-7.49 (m, 3H), 7.15-7.17 (m, 1H), 4.80 (d, 2H), 3.06 (hept., 1H), 2.62 (t, 1H), 1.36 (d, 6H).

MS: 397 (M+1)⁺

### EXAMPLE 35

### Thiophen-2-yl-[4-(4-isopropyl-phenyl)-6-propargyloxy-quinazolin-2-yl]-methanone

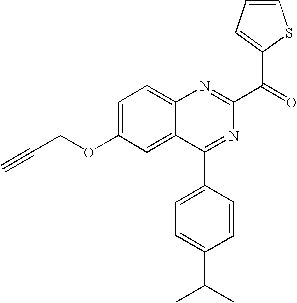

m. p. 113-117° C.

¹H-NMR (300 MHz, CDCl₃): 8.25-8.30 (m, 1H), 7.89 (d, 2H), 7.76 (dd, 2H), 7.65-7.71 (m, 2H), 7.46 (d, 2H), 7.20 (dd, 1H), 4.80 (d, 2H), 3.06 (hept., 1H), 2.63 (t, 1H), 1.36 (d, 6H).

MS: 413 (M+1)⁺

### EXAMPLE 36

### (3-Methyl-thiophen-2-yl-[4-(4-isopropyl-phenyl)-6-propargyloxy-quinazolin-2-yl]-methanone

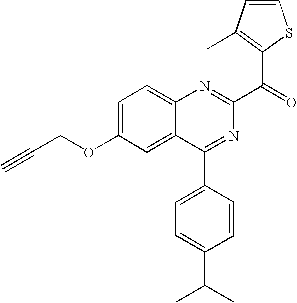

m. p. 130-132° C.

¹H-NMR (300 MHz, CDCl₃): 8.24 (d, 1H), 7.90 (d, 2H), 7.63-7.70 (m, 2H), 7.58 (d, 1H), 7.44 (d, 2H), 7.00 (d, 1H), 4.79 (d, 2H), 3.04 (hept., 1H), 2.71 (s, 3H), 2.61 (t, 1H), 1.34 (d, 6H).

MS: 427 (M+1)⁺

### EXAMPLE 37

### Benz[b]thiophen-2-yl-[4-(4-isopropyl-phenyl)-6-propargyloxy-quinazolin-2-yl]-methanone

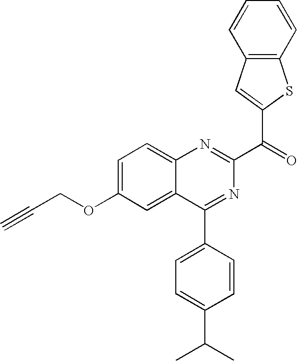

m. p. 167-169° C.

¹H-NMR (300 MHz, CDCl₃): 8.68 (s, 1H), 8.28-8.33 (m, 1H), 7.89-7.96 (m, 4H), 7.68-7.74 (m, 2H), 7.37-7.50 (m, 4H), 4.81 (d, 2H), 3.07 (hept., 1H), 2.64 (t, 1H), 1.37 (d, 6H).

MS: 463 (M+1)⁺

Preparation of benzo[b]thiophen-2-yl-oxo-acetaldehyde as described in EP 201 221.

### EXAMPLE 38

### Thiophen-3-yl-[4-(4-isopropyl-phenyl)-6-propargyloxy-quinazolin-2-yl]-methanone

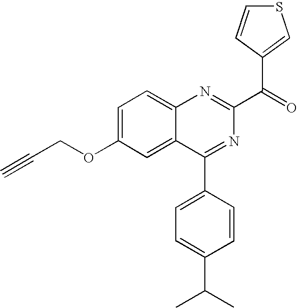

¹H-NMR (300 MHz, CDCl₃): 8.80-8.84 (dd, 1H), 8.20-8.25 (m, 1H), 7.94 (dd, 1H), 7.83 (d, 2H), 7.61-7.70 (m, 2H), 7.44 (d, 2H), 7.34 (dd, 1H), 4.80 (d, 2H), 3.04 (hept., 1H), 2.62 (t, 1H), 1.34 (d, 6H).

MS: 413 (M+1)⁺

### EXAMPLE 39

### (1-Methyl-1H-pyrrol)-2-yl-[4-(4-isopropyl-phenyl)-6-propargyloxy-quinazolin-2-yl]-methanone

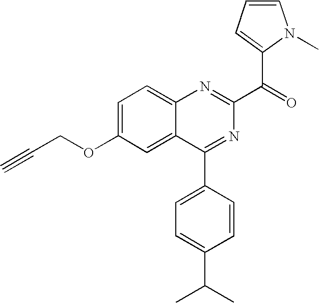

m. p. 126-128° C.

¹H-NMR (300 MHz, CDCl₃): 8.15-8.20 (m, 1H), 7.83 (d, 2H), 7.61-7.67 (m, 2H), 7.42 (d, 2H), 7.28 (dd, 1H), 6.94 (t, 1H), 6.18 (dd, 1H), 4.78 (d, 2H), 3.03 (hept., 1H), 4.12 (s, 3H), 2.61 (t, 1H), 1.33 (d, 6H).

MS: 410 (M+1)⁺

Preparation of (1-methyl-1H-pyrrol-2-yl)-oxo-acetaldehyde as described in EP 201 221.

### EXAMPLE 40

### 4-(4-Isopropyl-phenyl)-6-propargyloxy-quinazoline-2-carboxylic acid ethyl ester

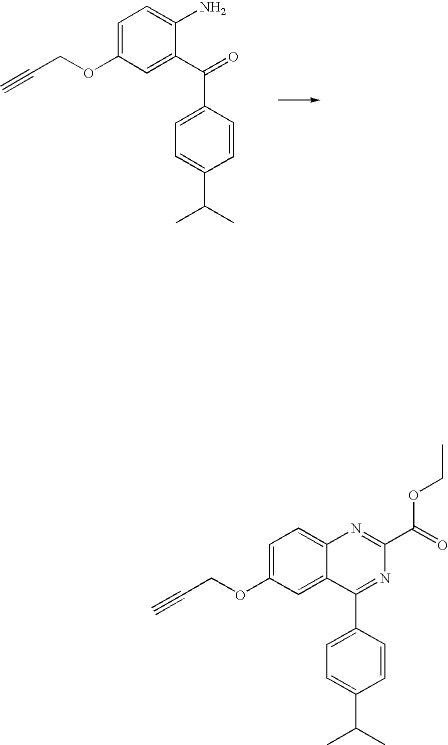

To a mixture of 2 g (6.8 mmol) (2-amino-5-propargyloxy-phenyl)-(4-isopropyl-phenyl)-methanone and 1.6 g ammonium acetate are added 7 ml water and 1.4 g (6.8 mmol) ethyl glyoxylate (50% in toluene). After vigorously stirring in the presence of air for 3 days the reaction mixture is extracted with water and CH₂Cl₂. The organic layers are dried over MgSO₄ and evaporated. Purification by flash chromatography (hexane/ethyl acetate) affords 4-(4-isopropyl-phenyl)-6-propargyloxy-quinazoline-2-carboxylic acid ethyl ester.

¹H-NMR (300 MHz, CDCl₃): 8.30 (d, 1H), 7.83 (d, 2H), 7.67 (dd, 1H), 7.65 (s, 1H), 7.43 (d, 2H), 4.79 (d, 2H), 4.60 (q, 2H), 3.04 (hept, 1H), 2.61 (t, 1H), 1.50 (t, 3H), 1.34 (d, 6H)

MS: 375 (M+1)⁺

### EXAMPLE 41

### [4-(4-isopropyl-phenyl)-6-propargyloxy-quinazolin-2-yl]-pyridine-3-yl-methanone

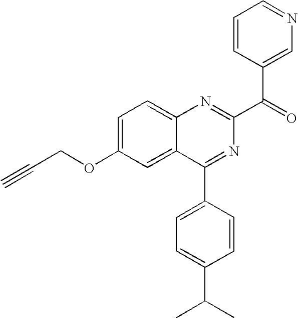

To a solution of 35 mg (0.085 mmol) of [4-(4-isopropyl-phenyl)-6-propargyloxy-quinazolin-2-yl]-pyridine-3-yl-methanol in 2 ml of acetone is added dropwise 49 μl (0.128 mmol) 2.6 M Jones reagent. An exothermic reaction takes place and the mixture turns dark. The oxidation reaction is complete after stirring for two hrs at rt. The chromium salts are filtered off and washed several times with acetone. After concentration i.V. the residue is distributed between ethyl acetate and water. Drying of the organic phase over anhydrous magnesium sulfate and evaporation of the solvent affords a yellow oil, which is purified by chromatography (dichloromethane/methanol). The product is obtained as a yellow solid.

¹H-NMR (400 MHz, CDCl₃): 9.46 (d, 1H), 8.82 (dd, 1H), 8.52-8.56 (m, 1H), 8.22 (dd, 1H), 7.84 (d, 2H), 7.68-7.71 (m, 2H), 7.42-7.49 (m, 3H), 4.81 (d, 2H), 3.03 (hept., 1H), 2.63 (t, 1H), 1.33 (d, 6H).

MS: 408 (M+1)⁺

Preparation of the starting material:

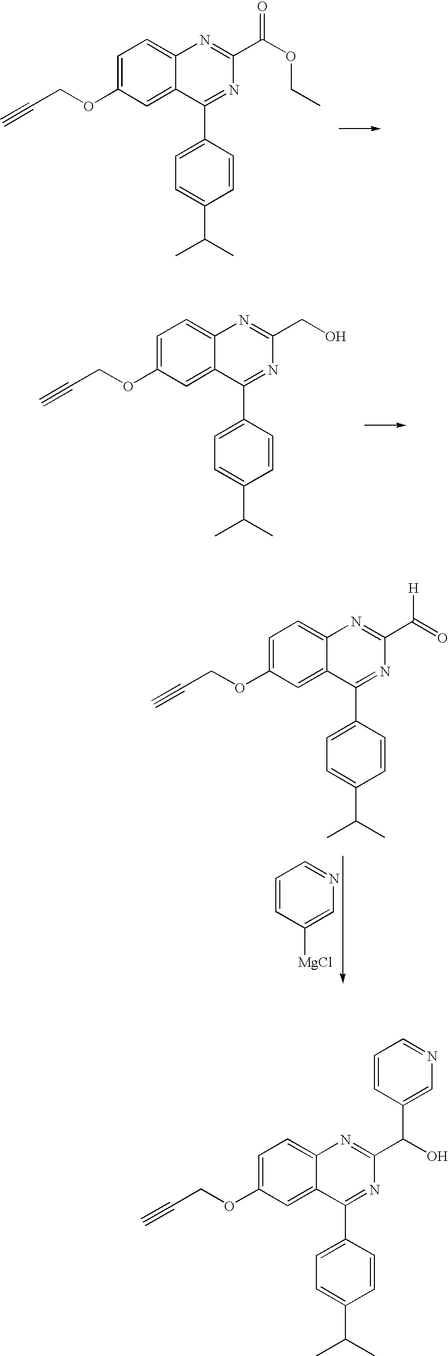

### A) [4-(4-Isopropyl-phenyl)-6-propargyloxy-quinazoline-2-yl-]methanol

A solution of 1.0 g (2.67 mmol) of 4-(4-isopropyl-phenyl)-6-propargyloxy-quinazoline-2-carboxylic acid ethyl ester in 20 ml THF is cooled with a water/ice bath and treated with 1.6 ml 1M lithium aluminum hydride solution. After complete addition the reaction mixture is quenched by pouring it into a saturated ammonium chloride/ethyl acetate solution. Extraction and concentration i.V. yields the product in the form of a yellow oil. The crude material is directly used in the following oxidation step.

### B) 4-(4-Isopropyl-phenyl)-6-propargyloxy-quinazoline-2-carbaldehyde

A solution of 3.9 g (11.7 mmol) of the alcohol prepared in step A in 40 ml dichloromethane is oxidized at rt with 1.1 eq Dess-Martin reagent. The mixture is filtered after stirring for 3 hrs. Distribution between ethyl acetate, water and sodium thiosulfate solution affords after concentration of the organic phases the crude aldehyde. This is purified by recrystallization from a mixture of ethyl acetate/hexanes to give a yellow-brown solid.

¹H-NMR (400 MHz, CDCl₃): 10.29 (s, 1H), 8.25 (d, 1H), 7.82 (d, 2H), 7.67-7.72 (m, 2H), 7.45 (d, 2H), 4.80 (d, 2H), 3.04 (hept., 1H), 2.62 (t, 1H), 1.33 (d, 6H)

### C) [4-(4-Isopropyl-phenyl)-6-propargyloxy-quinazolin-2-yl]-pyridine-3-yl-methanol

To a solution of 0.5 ml 2 M isopropyl magnesium chloride in THF is added dropwise 3-bromo-pyridine (80 mg in 0.5 ml THF) at 0° C. After the addition stirring is continued for another 30 minutes at rt, then the reaction mixture is cooled to −70° C. and the aldehyde obtained in step B is added (120 mg in 2 ml THF). The cooling bath is removed and the mixture is warmed to rt. Extraction with dichloromethane/water affords a yellow oil which is purified by chromatography (dichloromethane/methanol).

¹H-NMR (400 MHz, CDCl₃): 8.93 (d, 1H), 8.50 (dd, 1H), 8.03 (d, 1H), 7.93 (dd, 1H), 7.70-7.75 (m, 2H), 7.60-7.64 (m, 2H), 7.42 (d, 2H), 7.20-7.25 (m, 1H), 6.08 (d, 1H), 5.34 (d. 1H), 4.74-4.75 (m, 2H), 3.03 (hept., 1H), 2.58 (t, 1H), 1.34 (d, 6H)

The compounds of the following examples are prepared in an analogous manner using the appropriate starting materials:

### EXAMPLE 42

### [4-(4-Isopropyl-phenyl)-6-propargyloxy-quinazolin-2-yl]-naphthalen-1-yl-methanone

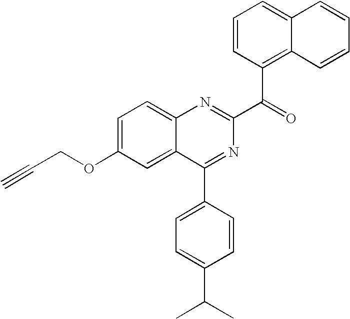

¹H-NMR (400 MHz, CDCl₃): 8.62 (d, 1H), 8.16 (d, 1H), 8.05 (d, 1H), 7.90-7.94 (m, 1H), 7.86 (dd, 1H), 7.76-7.80 (m, 2H), 7.69 (d, 1H), 7.65 (dd, 1H), 7.48-7.60 (m, 3H), 7.39 (d, 2H), 4.79 (d, 2H), 3.00 (hept., 1H), 2.62 (t, 1H), 1.30 (d, 6H).

MS: 457 (M+1)⁺

### EXAMPLE 43

### [4-(4-Isopropyl-phenyl)-6-propargyloxy-naphathalen-2-yl]-methanone

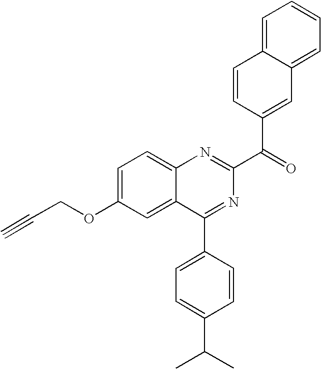

¹H-NMR (400 MHz, CDCl₃): 8.68 (br s, 1H), 8.24 (dd, 1H), 8.21 (d, 1H), 7.84-7.97 (m, 5H), 7.73 (d, 1H), 7.69 (dd, 1H), 7.59-7.63 (m, 1H), 7.51-7.56 (m, 1H), 7.43 (d, 2H), 4.81 (d, 2H), 3.02 (hept., 1H), 2.63 (t, 1H), 1.32 (d, 6H).

MS: 457 (M+1)⁺

### EXAMPLE 44

### Benzothiazol-2-yl-[4-(4-isopropyl-phenyl)-6-propargyloxy-quinazolin-2-yl]-methanone

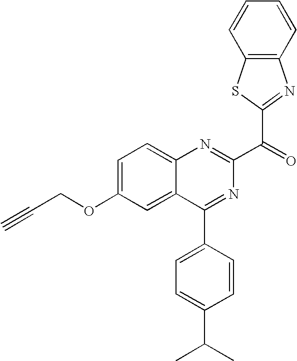

¹H-NMR (400 MHz, CDCl₃): 8.30-8.33 (m, 2H), 8.02-8.06 (m, 1H), 7.89-7.93 (m, 2H), 7.70-7.74 (m, 2H), 7.53-7.62 (m, 2H), 7.45-7.49 (m, 2H), 4.82 (d, 2H), 3.05 (hept., 1H), 2.63 (t, 1H), 1.35 (d, 6H).

MS: 457 (M+1)⁺

### EXAMPLE 45

### [4-(4-Isopropyl-phenyl)-6-propargyloxy-quinazolin-2-yl]-thiazol-5-yl-methanone

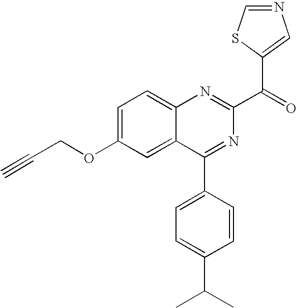

A mixture of 28.3 mg (0.18 mmol) 5-trimethylsilanyl-thiazole (preparation cf. J. Org. Chem. 1988, 53, 1748), 119 mg (0.36 mmol) 4-(4-isopropyl-phenyl)-6-propargyloxy-quinazoline-2-carbaldehyde and 27.3 mg (0.18 mmol) cesium fluoride in 2 ml THF is stirred for 3 days at 60° C. After evaporation the dark residue is chromatographed (hexane/ethyl acetate). The yellow oil obtained (alcohol) slowly transforms into the desired ketone on standing, which is obtained pure after another chromatographic purification.

¹H-NMR (400 MHz, CDCl₃): 9.26 (s, 1H), 9.11 (s, 1H), 8.35 (d, 1H), 7.94 (d, 2H), 7.74-7.81 (m, 2H), 7.53 (d, 2H), 4.84 (d, 2H), 3.10 (hept., 1H), 2.68 (t, 1H), 1.39 (d, 6H).

MS: 414 (M+1)⁺

### EXAMPLE 46

### [4-(4-Isopropyl-phenyl)-6-propargyloxy-quinazolin-2-yl]-piperidin-1-yl-methanone

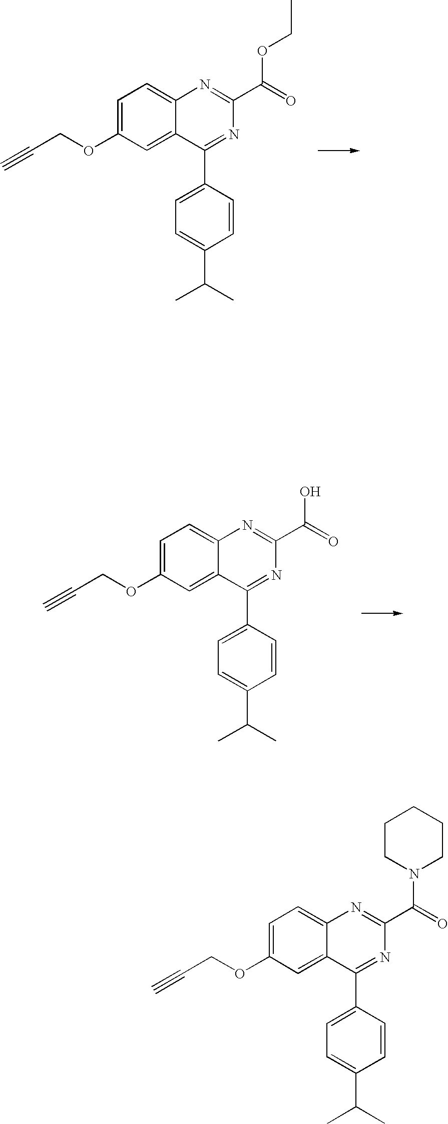

### A) 4-(4-Isopropyl-phenyl)-6-propargyloxy-quinazoline-2-carboxylic acid

A solution of 1.7 g (4.5 mmol) 4-(4-isopropyl-phenyl)-6-propargyloxy-quinazoline-2-carboxylic acid ethyl ester in 50 ml ethanol is treated at RT with 15 ml aqueous 1 M NaOH. After 1.5 h the reaction mixture is acidified with 1 M hydrochloric acid and extracted with CH₂Cl₂. The organic layers are dried over MgSO₄ and evaporated. The free acid is obtained after chromatography (hexane/ethyl acetate).

¹H-NMR (300 MHz, CDCl₃): 8.28 (d, 1H), 7.81 (d, 2H), 7.73 (dd, 1H), 7.71 (s, 1H), 7.47 (d, 2H), 4.81 (d, 2H), 3.06 (hept, 1H), 2.63 (t, 1H), 1.36 (d, 6H).

### B) [4-(4-Isopropyl-phenyl)-6-propargyloxy-quinazolin-2-yl]-piperidin-1-yl-methanone

A solution of 22 mg (64 μmol) of the acid prepared above, 6.3 μl (64 μmol) piperidine, 43 mg (96 μmol) BOP, and 16 μl (96 μmmol) Hünig's base in 0.5 ml THF is stirred overnight. The reaction mixture is acidified with 1 M hydrochloric acid and extracted with CH₂Cl₂. After drying over MgSO₄ and evaporation of the solvent the crude product is purified by preparative reversed phase HPLC.

¹H-NMR (300 MHz, DMSO-d₆): 8.04 (d, 1H), 7.80 (d, 2H), 7.74 (dd, 1H), 7.61 (d, 1H), 7.49 (d, 2H), 4.94 (d, 2H), 3.74 (t, 1H), 3.63 (m, 2H), 3.17 (m, 2H), 3.02 (hept, 1H), 1.60 (m, 4H), 1.47 (m, 2H), 1.28 (d, 6H).

MS: 414 (M+1)⁺

The compounds of the following examples are prepared in an analogous manner using the appropriate starting materials:

### EXAMPLE 47

### 4-(4-Isopropyl-phenyl)-6-prop-2-ynyloxy-quinazoline-2-carboxylic acid (3-chloro-phenyl)-amide

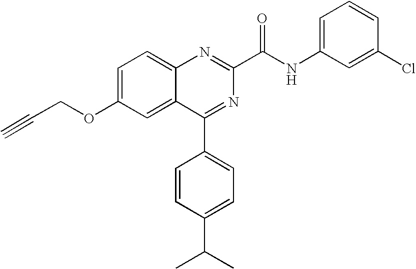

¹H-NMR (400 MHz, CDCl₃): 10.28 (s, 1H), 8.31 (d, 1H), 7.88 (s, 1H), 7.84 (d, 2H), 7.78 (d, 1H), 7.71-7.67 (m, 2H), 7.49 (d, 2H), 7.32 (t, 1H), 7.14 (d, 1H), 4.80 (d, 2H), 3.07 (hept, 1H), 2.62 (t, 1H), 1.37 (d, 6H).

MS: 456 (M+1)⁺ (isotope pattern for 1 Cl)

### EXAMPLE 48

### 4-(4-Isopropyl-phenyl)-6-prop-2-ynyloxy-quinazoline-2-carboxylic acid (3-methoxy-phenyl)-amide

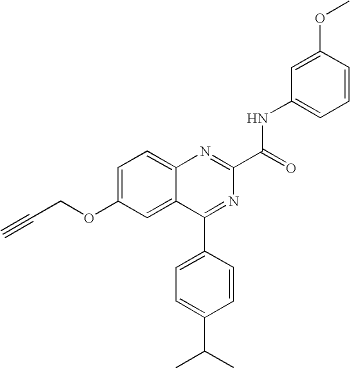

¹H-NMR (300 MHz, DMSO d₆): 10.66 (s, 1H), 8.23 (d, 1H), 7.95 (d, 2H), 7.84 (dd, 1H), 7.70 (d, 1H), 7.57 (m, 1H), 7.54 (d, 2H), 7.49 (dd, 1H), 7.29 (t, 1H), 6.73 (dd, 1H), 5.00 (d, 2H), 3.79 (t, 1H), 3.78 (s, 3H), 3.06 (hept, 1H), 1.32 (d, 6H).

MS: 452 (M+1)⁺

### EXAMPLE 49

### 4-(4-Isopropyl-phenyl)-6-prop-2-ynyloxy-3,4-dihydro-quinazoline-2-carboxylic acid (3-methylsulfanyl-phenyl)-amide

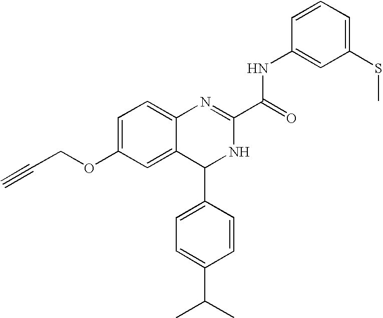

¹H-NMR (300 MHz, CDCl₃): 10.24 (s, 1H), 8.31 (d, 1H), 7.90 (t, 1H), 7.85 (d, 2H), 7.72-7.67 (m, 2H), 7.56 (dd, 1H), 7.50 (d, 2H), 7.30 (t, 1H), 7.06 (dd, 1H), 4.81 (d, 2H), 3.09 (hept, 1H), 2.64 (t, 1H), 2.55 (s, 3H), 1.39 (d, 6H).

MS: 470 (M+1)⁺

### EXAMPLE 50

### 4-(4-Isopropyl-phenyl)-6-prop-2-ynyloxy-3,4-dihydro-quinazoline-2-carboxylic acid (3-methanesulfonyl-phenyl)-amide

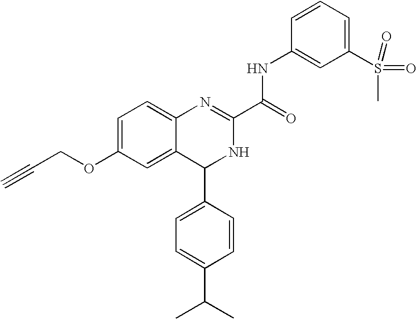

¹H-NMR (300 MHz, CDCl₃): 10.47 (s, 1H), 8.48 (d, broad, 1H), 8.32 (d, 1H), 8.16 (t, 1H), 7.85 (d, 2H), 7.75-7.61 (m, 4H), 7.51 (d, 2H), 4.81 (d, 2H), 3.12 (s, 3H), 3.10 (hept, 1H), 2.65 (t, 1H), 1.40 (d, 6H).

MS: 502 (M+1)⁺

### EXAMPLE 51

### 4-(4-Isopropyl-phenyl)-6-prop-2-ynyloxy-quinazoline-2-carboxylic acid (3-trifluoromethylsulfanyl-phenyl)-amide

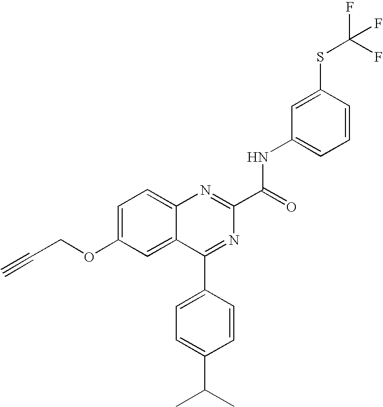

¹H-NMR (400 MHz, CDCl₃): 10.34 (s, 1H), 8.34 (d, 1H), 8.16 (dt, 1H), 8.05 (m, 1H), 7.86 (d, 2H), 7.72 (dd, 1H), 7.69 (d, 1H), 7.51 (d, 2H), 7.48-7.45 (m, 2H), 4.81 (d, 2H), 3.09 (hept, 1H), 2.64 (t, 1H), 1.39 (d, 6H).

MS: 522 (M+1)⁺

### EXAMPLE 52

### 4-(4-Isopropyl-phenyl)-6-prop-2-ynyloxy-3,4-dihydro-quinazoline-2-carboxylic acid (3-sulfamoyl-phenyl)-amide

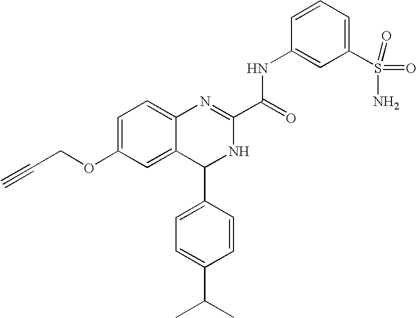

¹H-NMR (400 MHz, DMSO d₆): 8.49 (s, 1H), 8.25 (d, 1H), 8.08 (dt, 1H), 7.98 (d, 2H), 7.85 (dd, 1H), 7.72 (d, 1H), 7.62-7.58 (m, 2H), 7.56 (d, 2H), 5.01 (d, 2H), 3.80 (t, 1H), 3.06 hept, 1H), 1.32 (d, 6H).

MS: 503 (M+1)⁺

### EXAMPLE 53

### 4-(4-Isopropyl-phenyl)-6-prop-2-ynyloxy-3,4-dihydro-quinazoline-2-carboxylic acid [3-(2-hydroxy-ethanesulfonyl)-phenyl]amide

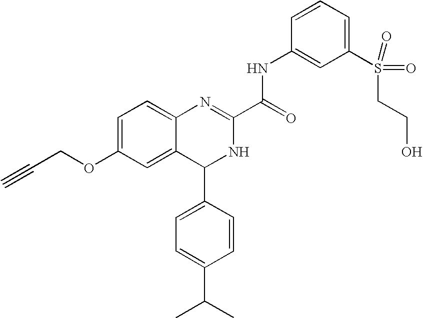

¹H-NMR (300 MHz, CDCl₃): 10.47 (s, 1H), 8.42 (d, broad, 1H), 8.33 (d, 1H), 8.21 (t, 1H), 7.85 (d, 2H), 7.75-7.62 (m, 4H), 7.52 (d, 2H), 4.82 (d, 2H), 4.07 (m, 1H), 3.43 (m, 1H), 3.10 (hept, 1H), 2.65 (t, 1H), 1.40 (d, 6H).

MS: 532 (M+1)⁺

### EXAMPLE 54

### 4-(4-Isopropyl-phenyl)-6-prop-2-ynyloxy-3,4-dihydro-quinazoline-2-carboxylic acid (5-ethanesulfonyl-2-hydroxy-phenyl)-amide

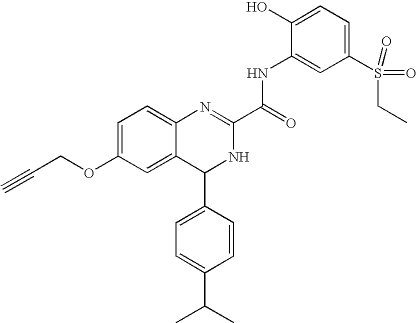

¹H-NMR (400 MHz, CDCl₃): 10.60 (s, 1H), 8.34 (d, 1H), 7.82 (d, 2H), 7.76-7.73 m, 2H), 7.71 (d, 1H), 7.67 (dd, 1H), 7.50 (d, 2H), 7.23 (d, H), 4.81 (d, 2H), 3.11 (q, 2H), 3.07 (hept, 1H), 2.63 (t, 1H), 1.37 (d, 6H), 1.28 (t, 3H).

MS: 532 (M+1)⁺

### EXAMPLE 55

### 4-(4-Isopropyl-phenyl)-6-prop-2-ynyloxy-quinazoline-2-carboxylic acid (3-nitro-phenyl)-amide

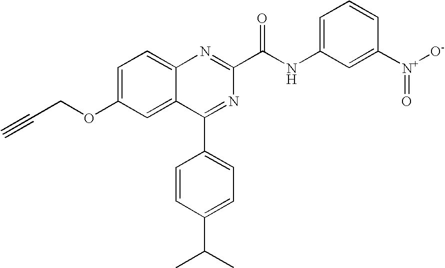

¹H-NMR (400 MHz, CDCl₃): 10.49 (s, 1H), 8.54 (t, 1H), 8.45 (d, 1H), 8.33 (d, 1H), 8.02 (dd, 1H), 7.85 (d, 2H), 7.70 (d, 1H), 7.73 (dd, 1H), 7.59 (t, 1H), 7.51 (d, 2H), 4.81 (d, 2H), 3.09 (hept, 1H), 2.63 (t, 1H), 1.38 (d, 6H).

MS: 467 (M+1)⁺

### EXAMPLE 56

### 4-(4-Isopropyl-phenyl)-6-prop-2-ynyloxy-quinazoline-2-carboxylic acid (3-cyano-phenyl)-amide

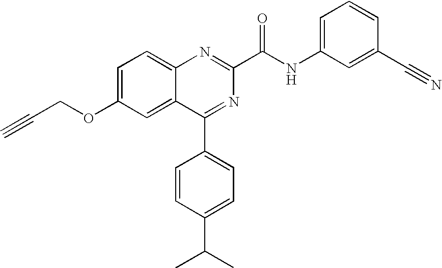

¹H-NMR (400 MHz, CDCl₃): 10.40 (s, 1H), 8.33 (d, 1H), 8.17 (m, 1H), 8.14 (m, 1H), 7.84 (d, 2H), 7.72 (dd, 1H), 7.69 (d, 1H), 7.53-7.49 (m, 3H), 7.45 (dt, 1H), 4.81 (d, 2H), 3.08 (hept, 1H), 2.63 (t, 1H), 1.38 (d, 6H).

MS: 447 (M+1)⁺

### EXAMPLE 57

### 3-{[4-(4-Isopropyl-phenyl)-6-prop-2-ynyloxy-quinazoline-2-carbonyl]-amino}-benzoic acid methyl ester

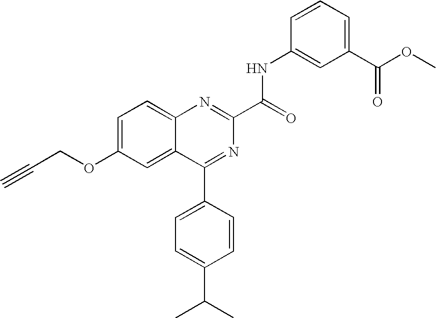

¹H-NMR (300 MHz, CDCl₃): 10.37 (s, 1H), 8.41 (dd, 1H), 8.32 (d, 1H), 8.21 (t, 1H), 7.86 (d, 2H), 7.83 (d, 1H), 7.72-7.68 (m, 2H), 7.51 (t, 1H), 7.51 (d, 2H), 4.81 (d, 2H), 3.96 (s, 3H), 3.10 (hept, 1H), 2.64 (t, 1H), 1.40 (d, 6H).

MS: 480 (M+1)⁺

### EXAMPLE 58

### 3-{[4-(4-Isopropyl-phenyl)-6-prop-2-ynyloxy-quinazoline-2-carbonyl]-amino}-benzoic acid ethyl ester

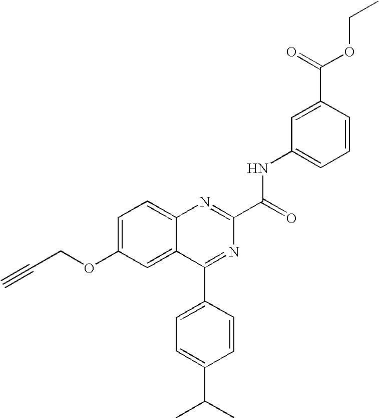

¹H-NMR (400 MHz, CDCl₃): 10.36 (s, 1H), 8.36 (dd, 1H), 8.28 (d, 1H), 8.22 (t, 1H), 7.87-7.84 (m, 1H), 7.83 (d, 2H), 7.72 (dd, 1H), 7.68 (d, 1H), 7.50 (t, 1H), 7.49 (d, 2H), 4.80 (d, 2H), 4.41 (q, 2H), 3.08 (hept, 1H), 2.62 (t, 1H), 1.42 (t, 3H), 1.37 (d, 6H).

MS: 494 (M+1)⁺

### EXAMPLE 59

### 3-{[4-(4-Isopropyl-phenyl)-6-prop-2-ynyloxy-quinazoline-2-carbonyl]-amino}-benzoic acid isopropyl ester

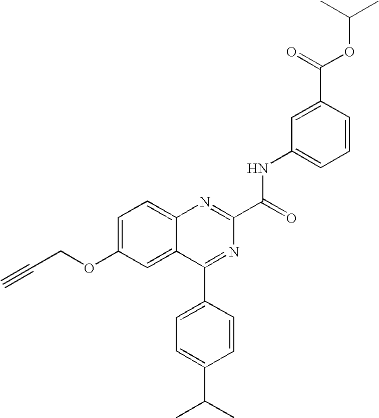

¹H-NMR (400 MHz, CDCl₃): 10.35 (s, 1H), 8.34 (ddd, 1H), 8.27 (d, 1H), 8.22 (t, 1H), 7.85 (dt, 1H), 7.83 (d, 2H), 7.71 (dd, 1H), 7.68 (d, 1H), 7.50 (t, 1H), 7.49 (d, 2H), 5.28 (hept, 1H), 4.80 (d, 2H), 3.08 (hept, 1H), 2.63 (t, 1H), 1.39 (d, 6H), 1.37 (d, 6H).

MS: 508 (M+1)⁺

### EXAMPLE 60

### 3-{[4-(4-Isopropyl-phenyl)-6-prop-2-ynyloxy-quinazoline-2-carbonyl]-amino}-benzoic acid tert-butyl ester

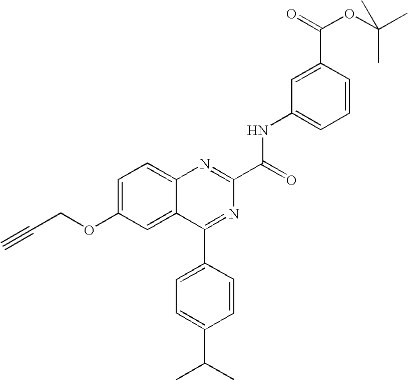

¹H-NMR (400 MHz, DMSO d₆): 10.94 (s, 1H), 8.50 (t, 1H), 8.24 (d, 1H), 8.13 (d, broad, 1H), 7.97 (d, 2H), 7.85 (dd, 1H), 7.71 (d, 1H), 7.67 (d, 1H), 7.55 (d, 2H), 7.52 (t, 1H), 5.01 (d, 2H), 3.80 (t, 1H), 3.06 (hept, 1H), 1.57 (s, 9H), 1.31 (d, 6H).

MS: 522 (M+1)⁺

### EXAMPLE 61

### 4-(4-Isopropyl-phenyl)-6-prop-2-ynyloxy-quinazoline-2-carboxylic acid (3-carbamoyl-phenyl)-amide

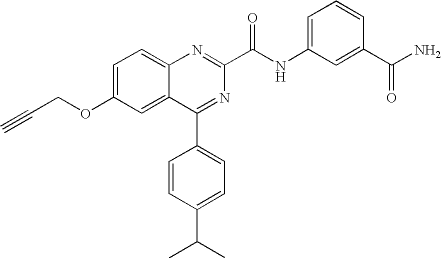

¹H-NMR (400 MHz, CDCl₃): 10.41 (s, 1H), 8.31 (d, 1H), 8.26 (t, 1H), 8.15 (d, 1H), 7.84 (d, 2H), 7.70 (dd, 1H), 7.68 (d, 1H), 7.62 (d, 1H), 7.50 (t, 1H), 7.49 (d, 2H), 6.32 (broad, 1H), 5.69 (broad, 1H), 4.80 (d, 2H), 3.08 (hept, 1H), 2.63 (t, 1H), 1.38 (d, 6H).

MS: 465 (M+1)⁺

### EXAMPLE 62

### 4-(4-Isopropyl-phenyl)-6-prop-2-ynyloxy-quinazoline-2-carboxylic acid (3-acetyl-phenyl)-amide

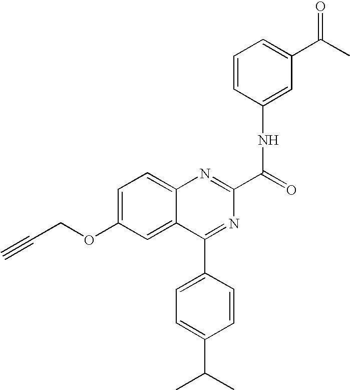

¹H-NMR (400 MHz, CDCl₃): 10.38 (s, 1H), 8.30-8.26 (m, 3H), 7.84 (d, 2H), 7.76 (dt, 1H), 7.71 (dd, 1H), 7.68 (d, 1H), 7.52 (t, 1H), 7.50 (d, 2H), 4.80 (d, 2H), 3.08 (hept, 1H), 2.66 (s, 3H), 2.62 (t, 1H), 1.38 (d, 6H).

MS: 464 (M+1)⁺

### EXAMPLE 63

### 3-{[4-(4-Isopropyl-phenyl)-6-prop-2-ynyloxy-quinazoline-2-carbonyl]-amino}-5-methoxy-benzoic acid methyl ester

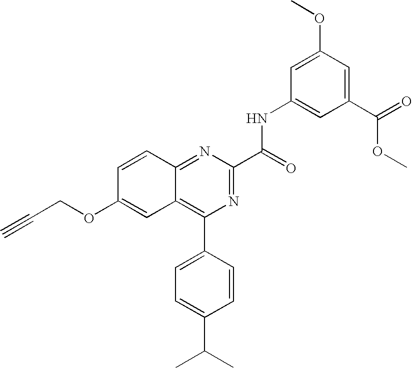

¹H-NMR (400 MHz, CDCl₃): 10.34 (s, 1H), 8.31 (d, 1H), 8.18 (s, 1H), 7.85 (d, 2H), 7.71-7.68 (m, 3H), 7.49 (d, 2H), 7.37 (m, 1H), 4.80 (d, 2H), 3.93 (s, 3H), 3.90 (s, 3H), 3.08 (hept, 1H), 2.62 (t, 1H), 1.38 (d, 6H).

MS: 510 (M+1)⁺

### EXAMPLE 64

### 4-(4-Isopropyl-phenyl)-6-prop-2-ynyloxy-quinazoline-2-carboxylic acid (3-methylcarbamoyl-phenyl)-amide

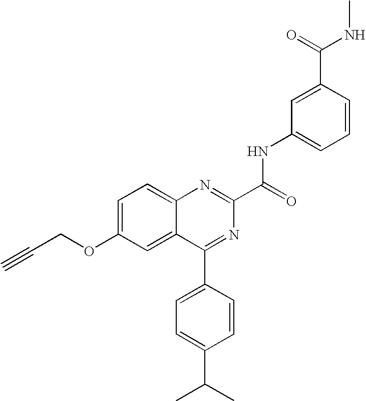

¹H-NMR (400 MHz, CDCl₃): 10.37 (s, 1H), 8.27 (d, 1H), 8.24 (s, 1H), 8.01 (d, 1H), 7.83 (d, 2H), 7.70 (dd, 1H), 7.68 (m, 1H), 7.57 (d, 1H), 7.48 (d, 2H), 7.44 (t, 1H), 6.62 (broad, 1H), 4.80 (d, 2H), 3.07 (hept, 1H), 3.05 (d, 3H), 2.63 (t, 1H), 1.37 (d, 6H).

MS: 479 (M+1)⁺

### EXAMPLE 65

### 4-(4-Isopropyl-phenyl)-6-prop-2-ynyloxy-quinazoline-2-carboxylic acid (3-tert-butylcarbamoyl-phenyl)-amide

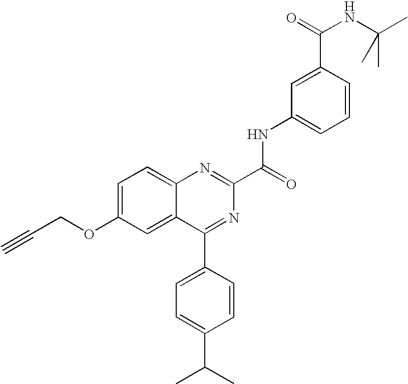

¹H-NMR (400 MHz, CDCl₃): 10.37 (s, 1H), 8.29 (d, 1H), 8.20 (t, 1H), 8.04 (d, broad, 1H), 7.83 (d, 2H), 7.70 (dd, 1H), 7.68 (d, 1H), 7.53 (d, broad, 1H), 7.49 (d, 2H), 7.45 (t, 1H), 6.14 (s, 1H), 4.80 (d, 2H), 3.07 (hept, 1H), 2.62 (t, 1H), 1.48 (s, 9H), 1.37 (d, 6H).

MS: 521 (M+1)⁺

### EXAMPLE 66

### 4-(4-Isopropyl-phenyl)-6-prop-2-ynyloxy-quinazoline-2-carboxylic acid (3-dimethylcarbamoyl-5-trifluoromethyl-phenyl)-amide

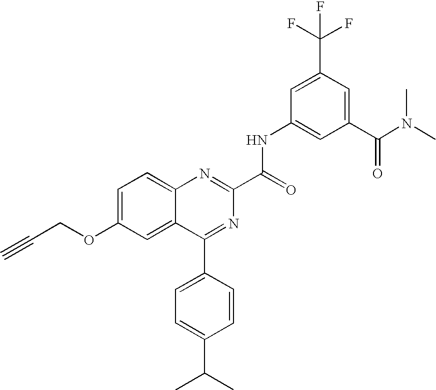

¹H-NMR (400 MHz, CDCl₃): 10.48 (s, 1H), 8.33 (d, 1H), 8.26 (s, 1H), 8.10 (s, 1H), 7.86 (d, 2H), 7.74 (dd, 1H), 7.71 (d, 1H), 7.53 (s, 1H), 7.51 (d, 2H), 4.82 (d, 2H), 3.17 (s, 3H), 3.11 (s, 3H), 3.09 (hept, 1H), 2.64 (t, 1H), 1.38 (d, 6H).

MS: 561 (M+1)⁺

### EXAMPLE 67

### 3-{[4-(4-Isopropyl-phenyl)-6-prop-2-ynyloxy-quinazoline-2-carbonyl]-amino}-5-trifluoromethyl-benzoic acid methyl ester

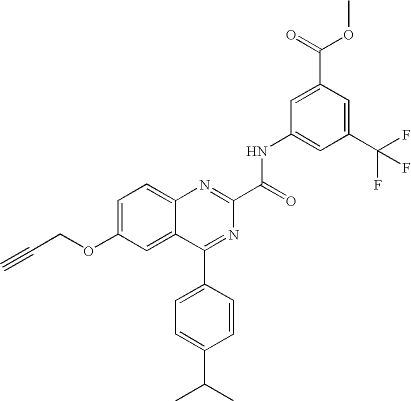

¹H-NMR (400 MHz, DMSO d₆): 11.33 (s, 1H), 8.92 (s, 1H), 8.68 (s, 1H), 8.27 (d, 1H), 7.99-7.97 (m, 3H), 7.87 (dd, 1H), 7.73 (d, 1H), 7.57 (d, 2H), 5.02 (d, 2H), 3.95 (s, 3H), 3.81 (t, 1H), 3.08 (hept, 1H), 1.33 (d, 6H).

MS: 548 (M+1)⁺

### EXAMPLE 68

### 3-{[4-(4-Isopropyl-phenyl)-6-prop-2-ynyloxy-quinazoline-2-carbonyl]-amino}-5-trifluoromethyl-benzoic acid isopropyl ester

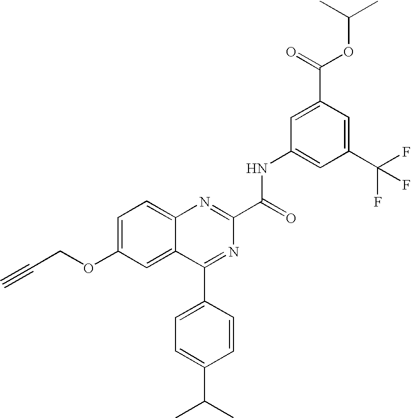

¹H-NMR (400 MHz, CDCl₃): 10.45 (s, 1H), 8.52 (m, 2H), 8.28 (d, 1H), 8.08 (m, 1H), 7.83 (d, 2H), 7.73 (dd, 1H), 7.69 (d, 1H), 7.50 (d, 2H), 5.30 (hept, 1H), 4.81 (d, 2H), 3.08 (hept, 1H), 2.63 (t, 1H), 1.42 (d, 6H), 1.38 (d, 6H).

MS: 576 (M+1)⁺

### EXAMPLE 69

### 2-Fluoro-5-{[4-(4-isopropyl-phenyl)-6-prop-2-ynyloxy-quinazoline-2-carbonyl]-amino}-benzoic acid methyl ester

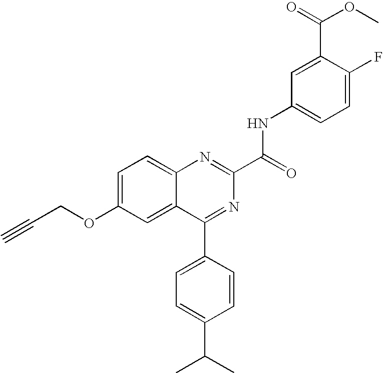

¹H-NMR (400 MHz, CDCl₃): 10.33 (s, 1H), 8.38-8.34 (m, 1H), 8.31 (d, 1H), 8.10 (dd, 1H), 7.83 (d, 2H), 7.71 (dd, 1H), 7.68 (d, 1H), 7.49 (d, 2H), 7.21 (t, 1H), 4.80 (d, 2H), 3.96 (s, 3H), 3.08 (hept, 1H), 2.62 (t, 1H), 1.37 (d, 6H).

MS: 498 (M+1)⁺

### EXAMPLE 70

### 2-Fluoro-5-{[4-(4-isopropyl-phenyl)-6-prop-2-ynyloxy-quinazoline-2-carbonyl]-amino}-benzoic acid isopropyl ester

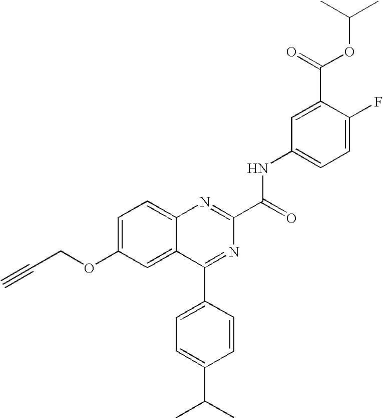

¹H-NMR (400 MHz, CDCl₃): 10.30 (s, 1H), 8.30-8.26 (m, 2H), 8.11 (dd, 1H), 7.82 (d, 2H), 7.72 (dd, 1H), 7.68 (d, 1H), 7.49 (d, 2H), 7.18 (t, 1H), 5.29 (hept, 1H), 4.80 (d, 2H), 3.07 (hept, 1H), 2.63 (t, 1H), 1.40 (d, 6H), 1.37 (d, 6H).

MS: 526 (M+1)⁺

### EXAMPLE 71

### 2-Chloro-5-{[4-(4-isopropyl-phenyl)-6-prop-2-ynyloxy-quinazoline-2-carbonyl]-amino}-benzoic acid methyl ester

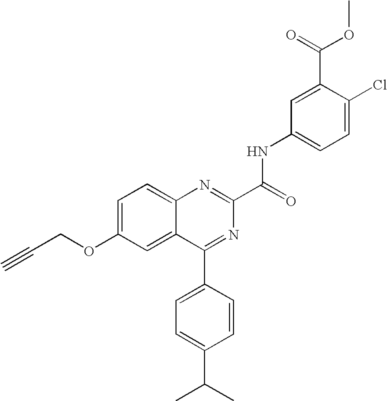

¹H-NMR (400 MHz, DMSO d₆): 11.06 (s, 1H), 8.46 (d, 1H), 8.25 (d, 1H), 8.16 (dd, 1H), 7.97 (d, 2H), 7.86 (dd, 1H), 7.72 (d, 1H), 7.63 (d, 1H), 7.56 (d, 2H), 5.02 (d, 2H), 3.91 (s, 3H), 3.80 (t, 1H)), 3.07 (hept, 1H), 1.32 (d, 6H).

MS: 514 (M+1)⁺ (isotope pattern for 1 Cl)

### EXAMPLE 72

### 2,5-Dichloro-3-{[4-(4-isopropyl-phenyl)-6-prop-2-ynyloxy-quinazoline-2-carbonyl]-amino}-benzoic acid methyl ester

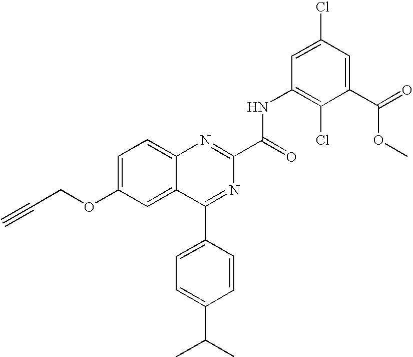

¹H-NMR (400 MHz, CDCl₃): 11.39 (s, 1H), 9.05 (d, 1H), 8.32 (d, 1H), 7.91 (d, 2H), 7.76 (d, 1H), 7.71 (dd, 1H), 7.61 (d, 1H), 7.47 (d, 2H), 4.82 (d, 2H), 3.96 (s, 3H), 3.06 (hept, 1H), 2.64 (t, 1H), 1.36 (d, 6H).

MS: 548 (M+1)⁺ (isotope pattern for 2 Cl)

### EXAMPLE 73

### 2,5-Dichloro-3-{[4-(4-isopropyl-phenyl)-6-prop-2-ynyloxy-quinazoline-2-carbonyl]-amino}-benzoic acid isopropyl ester

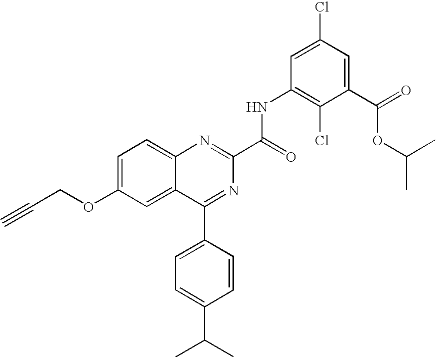

¹H-NMR (400 MHz, CDCl₃): 11.36 (s, 1H), 9.03 (d, 1H), 8.34 (d, 1H), 7.91 (d, 2H), 7.76 (d, 1H), 7.71 (dd, 1H), 7.54 (d, 1H), 7.47 (d, 2H), 5.29 (hept, 1H), 4.82 (d, 2H), 3.06 (hept, 1H), 2.64 (t, 1H), 1.41 (d, 6H), 1.36 (d, 6H).

MS: 548 (M+1)⁺ (isotope pattern for 2 Cl)

### EXAMPLE 74

### 4-(4-Isopropyl-phenyl)-6-prop-2-ynyloxy-quinazoline-2-carboxylic acid (3-cyano-5-fluoro-phenyl)-amide

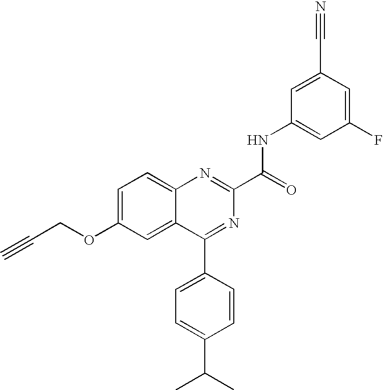

¹H-NMR (400 MHz, CDCl₃): 10.44 (s, 1H), 8.29 (d, 1H), 8.12 (dt, 1H), 7.83-7.81 (m, 3H), 7.73 (dd, 1H), 7.69 (d, 1H), 7.49 (d, 2H), 7.15 (ddd, 1H), 4.81 (d, 2H), 3.08 (hept, 1H), 2.63 (t, 1H), 1.37 (d, 6H).

MS: 465 (M+1)⁺

### EXAMPLE 75

### 4-(4-Isopropyl-phenyl)-6-prop-2-ynyloxy-quinazoline-2-carboxylic acid (3,4-dicyano-phenyl)-amide

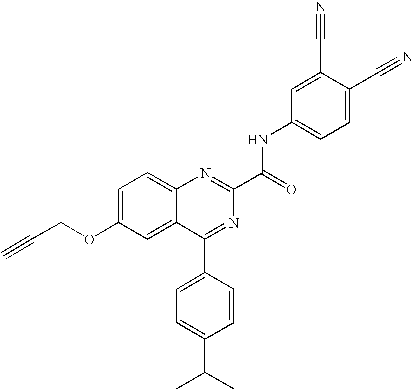

¹H-NMR (400 MHz, CDCl₃): 10.61 (s, 1H), 8.33 (d, 1H), 8.28 (d, 1H), 8.25 (dd, 1H), 7.81 (d, 1H), 7.81 (d, 2H), 7.74 (dd, 1H), 7.69 (d, 1H), 7.49 (d, 2H), 4.81 (d, 2H), 3.08 (hept, 1H), 2.63 (t, 1H), 1.37 (d, 6H).

MS: 472 (M+1)⁺

### EXAMPLE 76

### 4-(4-Isopropyl-phenyl)-6-prop-2-ynyloxy-quinazoline-2-carboxylic acid (4-cyano-3-trifluoromethyl-phenyl)-amide

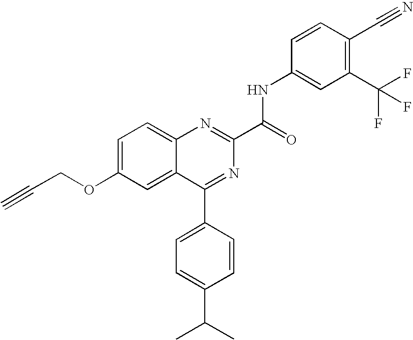

¹H-NMR (400 MHz, CDCl₃): 10.57 (s, 1H), 8.34 (dd, 1H), 8.28 (d, 1H), 8.16 (d, 1H), 7.87 (d, 1H), 7.81 (d, 2H), 7.75 (dd, 1H), 7.70 (d, 1H), 7.50 (d, 2H), 4.81 (d, 2H), 3.08 (hept, 1H), 2.63 (t, 1H), 1.37 (d, 6H).

MS: 515 (M+1)⁺

### EXAMPLE 77

### 4-(4-Isopropyl-phenyl)-6-prop-2-ynyloxy-quinazoline-2-carboxylic acid (3-trifluoromethyl-phenyl)-amide

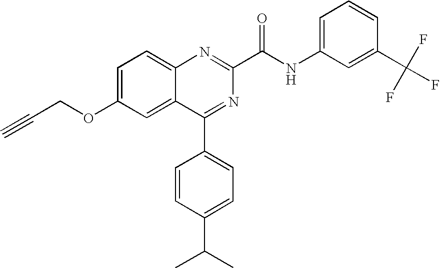

¹H-NMR (400 MHz, CDCl₃): 10.38 (s, 1H), 8.32 (d, 1H), 8.20 (d, 1H), 8.01 (s, 1H), 7.85 (d, 2H), 7.71 (dd, 1H), 7.69 (d, 1H), 7.54 (t, 1H), 7.50 (d, 2H), 7.42 (d, 1H), 4.80 (d, 2H), 3.08 (hept, 1H), 2.63 (t, 1H), 1.38 (d, 6H).

MS: 490 (M+1)⁺

### EXAMPLE 78

### 4-(4-Isopropyl-phenyl)-6-prop-2-ynyloxy-quinazoline-2-carboxylic acid (4-acetylamino-3-trifluoromethyl-phenyl)-amide

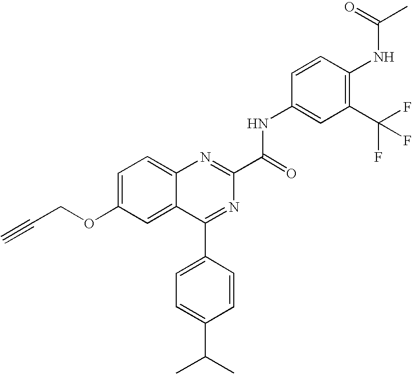

¹H-NMR (400 MHz, CDCl₃): 10.32 (s, 1H), 8.27 (d, 1H), 8.21 (s, 1H), 8.12 (d, 1H), 7.97 (d, 1H), 7.83 (d, 2H), 7.71 (dd, 1H), 7.67 (d, 1H), 7.49 (d, 2H), 7.44 (s, 1H), 4.80 (d, 2H), 3.07 (hept, 1H), 2.62 (t, 1H), 2.25 (t, 1H), 1.37 (d, 6H).

MS: 547 (M+1)⁺

### EXAMPLE 79

### 4-(4-Isopropyl-phenyl)-6-prop-2-ynyloxy-quinazoline-2-carboxylic acid (3-methoxy-5-trifluoromethyl-phenyl)-amide

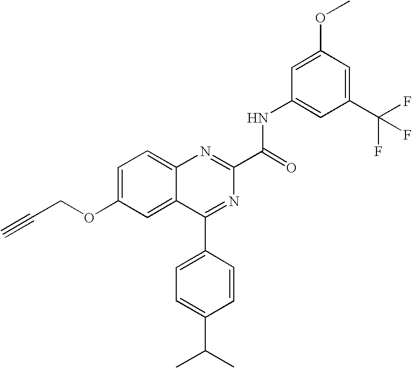

¹H-NMR (400 MHz, CDCl₃): 10.33 (s, 1H), 8.30 (d, 1H), 8.01 (t, 1H), 7.84 (d, 2H),7.70 (dd, 1H), 7.68 (d, 1H), 7.49 (d, 2H), 7.41 (s, 1H), 6.94 (s, 1H), 4.80 (d, 2H), 3.90 (s, 3H), 3.08 (hept, 1H), 2.62 (t, 1H), 1.37 (d, 6H).

MS: 520 (M+1)⁺

### EXAMPLE 80

### 4-(4-Isopropyl-phenyl)-6-prop-2-ynyloxy-quinazoline-2-carboxylic acid (3,5-bis-trifluoromethyl-phenyl)-amide

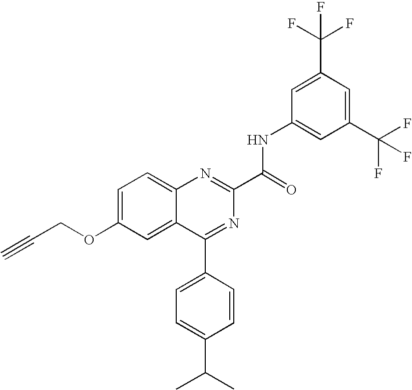

¹H-NMR (400 MHz, CDCl₃): 10.48 (s, 1H), 8.33 (s, 2H), 8.24 (d, 1H), 7.81 (d, 2H), 7.74 (dd, 1H), 7.69-7.68 (m, 2H), 7.50 (d, 2H), 4.81 (d, 2H), 3.08 (hept, 1H), 2.63 (t, 1H), 1.37 (d, 6H).

MS: 558 (M+1)⁺

### EXAMPLE 81

### 4-(4-Isopropyl-phenyl)-6-prop-2-ynyloxy-quinazoline-2-carboxylic acid (3-fluoro-5-trifluoromethyl-phenyl)-amide

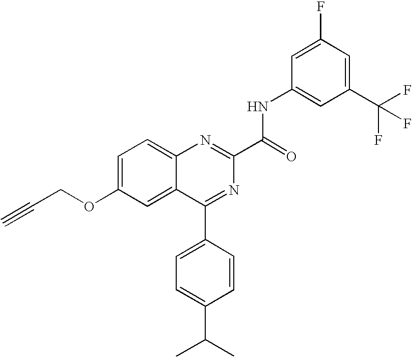

¹H-NMR (400 MHz, CDCl₃): 10.42 (s, 1H), 8.27 (d, 1H), 8.14 (d, 1H), 7.82 (d, 2H), 7.72 (dd, 1H), 7.68 (d, 1H), 7.66 (s, 1H), 7.50 (d, 2H), 7.13 (d, 1H), 4.80 (d, 2H), 3.08 (hept, 1H), 2.63 (t, 1H), 1.37 (d, 6H).

MS: 508 (M+1)⁺

### EXAMPLE 82

### 4-(4-Isopropyl-phenyl)-6-prop-2-ynyloxy-quinazoline-2-carboxylic acid (4-fluoro-3-trifluoromethyl-phenyl)-amide

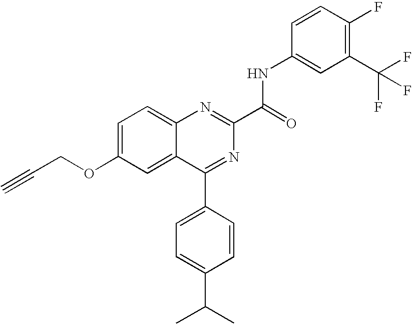

¹H-NMR (400 MHz, CDCl₃): 10.34 (s, 1H), 8.33 (d, 1H), 8.20 (dt, 1H), 8.01 (dd, 1H), 7.85 (d, 2H), 7.72 (dd, 1H), 7.69 (d, 1H), 7.51 (d, 2H), 7.26 (t, 1H), 4.81 (d, 2H), 3.09 (hept, 1H), 2.64 (t, 1H), 1.39 (d, 6H).

MS: 508 (M+1)⁺

### EXAMPLE 83

### 3-{[4-(4-Isopropyl-phenyl)-6-prop-2-ynyloxy-quinazoline-2-carbonyl]-amino}-2-methyl-benzoic acid methyl ester

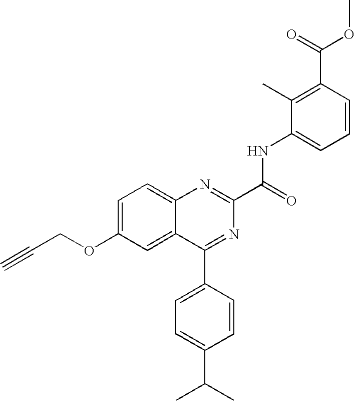

¹H-NMR (400 MHz, DMSO d₆): 10.65 (s, 1H), 8.24 (d, 1H), 8.00 (d, 2H), 7.93 (d, 1H), 7.85 (dd, 1H), 7.74 (d, 1H), 7.64 (d, 1H), 7.55 (d, 2H), 7.39 (t, 1H), 5.01 (d, 2H), 3.86 (s, 3H), 3.80 (t, 1H), 3.06 (hept, 1H), 2.47 (s, 3H), 1.31 (d, 6H).

MS: 494 (M+1)⁺

### EXAMPLE 84

### 3-{[4-(4-Isopropyl-phenyl)-6-prop-2-ynyloxy-quinazoline-2-carbonyl]-amino}-4-methyl-benzoic acid methyl ester

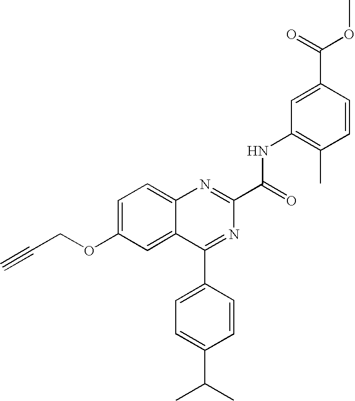

¹H-NMR (400 MHz, CDCl₃): 10.37 (s, 1H), 8.93 (s, 1H), 8.33 (d, 1H), 7.88 (d, 2H), 7.81 (dd, 1H), 7.72-7.69 (m, 2H), 7.49 (d, 2H), 7.32 (d, 1H), 4.82 d, 2H), 3.92 (s, 3H), 3.08 (hept, 1H), 2.64 (t, 1H), 2.50 (s, 1H), 1.38 (d, 6H).

MS: 494 (M+1)⁺

### EXAMPLE 85

### 3-{[4-(4-Isopropyl-phenyl)-6-prop-2-ynyloxy-quinazoline-2-carbonyl]-amino}-4-methoxy-benzoic acid methyl ester

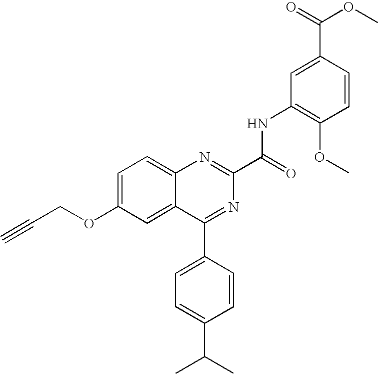

¹H-NMR (400 MHz, CDCl₃): 10.87 (s, 1H), 9.34 (d, 1H), 8.32 (d, 1H), 7.92 (d, 2H), 7.85 (dd, 1H), 7.72 (d, 1H), 7.68 (dd, 1H), 7.47 (d, 2H), 6.96 (d, 1H), 4.80 (t, 2H), 4.03 (s, 3H), 3.90 (s, 3H), 3.07 (hept, 1H), 2.63 (t, 1H), 1.37 (d, 6H).

MS: 510 (M+1)⁺

### EXAMPLE 86

### 5-{[4-(4-Isopropyl-phenyl)-6-prop-2-ynyloxy-quinazoline-2-carbonyl]-amino}-isophthalic acid dimethyl ester

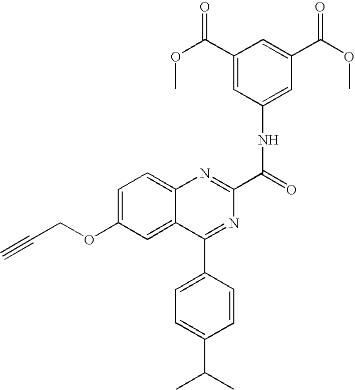

¹H-NMR (400 MHz, CDCl₃): 10.43 (s, 1H), 8.71 (d, 2H), 8.48 (t, 1H), 8.29 (d, 1H), 7.84 (d, 2H), 7.71 (dd, 1H), 7.68 (d, 1H), 7.50 (d, 2H), 4.80 (d, 2H), 3.97 (s, 6H), 3.08 (hept, 1H), 2.62 (t, 1H), 1.38 (d, 6H).

MS: 538 (M+1)⁺

### EXAMPLE 87

### 4-{[4-(4-Isopropyl-phenyl)-6-prop-2-ynyloxy-quinazoline-2-carbonyl]-amino}-phthalic acid dimethyl ester

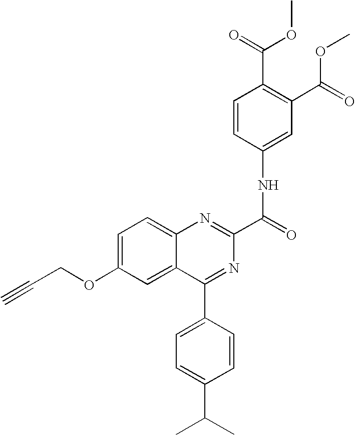

¹H-NMR (400 MHz, CDCl₃): 10.47 (s, 1H), 8.29 (d, 1H), 8.25 (dd, 1H), 7.92 (d, 1H), 7.88 (d, 1H), 7.83 (d, 2H), 7.70 (dd, 1H), 7.68 (d, 1H), 7.49 (d, 2H), 4.80 (d, 2H), 3.93 (s, 3H), 3.90 (s, 3H), 3.07 (hept, 1H); 2.62 (t, 1H), 1.37 (d, 6H).

MS: 538 (M+1)⁺

### EXAMPLE 88

### 4-(4-Isopropyl-phenyl)-6-prop-2-ynyloxy-quinazoline-2-carboxylic acid (3,5-dichloro-phenyl)-amide

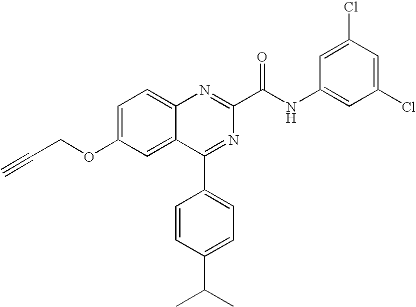

¹H-NMR (400 MHz, CDCl₃): 10.29 (s, 1H), 8.29 (d, 1H), 7.83 (d, 2H), 7.81 (d, 2H), 7.70 (dd, 1H), 7.67 (d, 1H), 7.49 (d, 2H), 7.15 (t, 1H), 4.80 (d, 2H), 3.07 (hept, 1H), 2.62 (t, 1H), 1.37 (d, 6H).

MS: 490 (M+1)⁺ (isotope pattern for 2 Cl)

### EXAMPLE 89

### 4-(4-Isopropyl-phenyl)-6-prop-2-ynyloxy-quinazoline-2-carboxylic acid (3,4-dichloro-phenyl)-amide

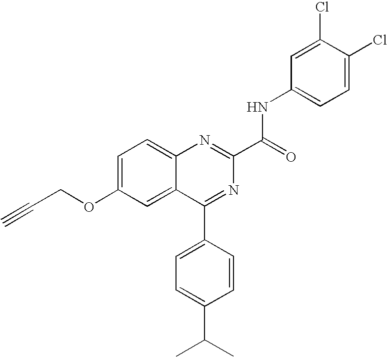

¹H-NMR (300 MHz, CDCl₃): 10.29 s, 1H), 8.32 (d, 1H), 8.02 (d, 1H), 7.85 (d, 2H), 7.77 (dd, 1H), 7.71 (dd, 1H), 7.68 (d, 1H), 7.50 (d, 2H), 7.46 (d, 1H), 4.81 (d, 2H), 3.10 (hept, 1H), 2.64 (t, 1H), 1.40 (d, 6H).

MS: 490 (M+1)⁺ (isotope pattern for 2 Cl)

### EXAMPLE 90

### 4-(4-Isopropyl-phenyl)-6-prop-2-ynyloxy-quinazoline-2-carboxylic acid (3-chloro-4-fluoro-phenyl)-amide

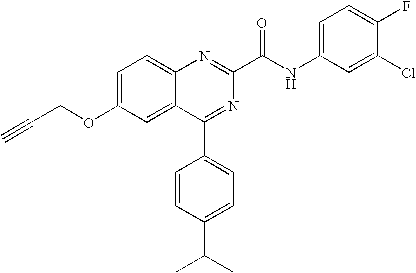

¹H-NMR (400 MHz, CDCl₃): 10.25 (s, 1H), 8.31 (d, 1H), 7.97 (d, 1H), 7.84 (d, 2H), 7.77-7.68 (m, 3H), 7.50 (d, 2H), 7.18 (t, 1H), 4.81 (d, 2H), 3.09 (hept, 1H), 2.63 (t, 1H), 1.38 (d, 6H).

MS: 474 (M+1)⁺ (isotope pattern for 1 Cl)

### EXAMPLE 91

### 4-(4-Isopropyl-phenyl)-6-prop-2-ynyloxy-quinazoline-2-carboxylic acid (4-chloro-3-trifluoromethyl-phenyl)-amide

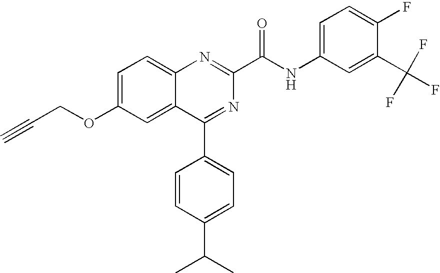

¹H-NMR (400 MHz, CDCl₃): 10.37 (s, 1H), 8.32 (d, 1H), 8.18 (dd, 1H), 8.07 (d, 1H), 7.84 (d, 2H), 7.72 (dd, 1H), 7.68 (d, 1H), 7.54 (d, 1H), 7.50 (d, 2H), 4.80 (d, 2H), 3.08 (hept, 1H), 2.63 (t, 1H), 1.38 (d, 6H).

MS: 524 (M+1)⁺ (isotope pattern for 1 Cl)

### EXAMPLE 92

### 5-{[4-(4-Isopropyl-phenyl)-6-prop-2-ynyloxy-quinazoline-2-carbonyl]-amino}-pyridine-2-carboxylic acid methyl ester

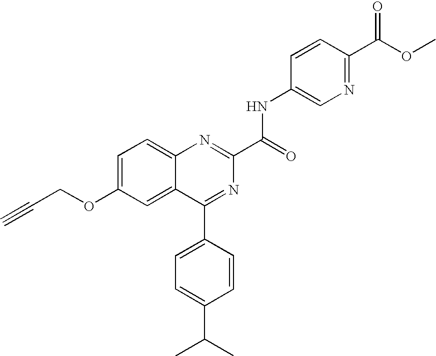

¹H-NMR (400 MHz, CDCl₃): 10.48 (s, 1H), 8.91 (d, 1H), 8.75 (dd, 1H), 8.31 (d, 1H), 8.22 (d, 1H), 7.84 (d, 2H), 7.72 (dd, 1H), 7.68 (d, 1H), 7.49 (d, 2H), 4.80 (d, 2H), 4.01 (s, 3H), 3.07 (hept, 1H), 2.62 (t, 1H), 1.37 (d, 6H).

MS: 481 (M+1)⁺

### EXAMPLE 93

### 5-{[4-(4-Isopropyl-phenyl)-6-prop-2-ynyloxy-quinazoline-2-carbonyl]-amino}-nicotinic acid methyl ester

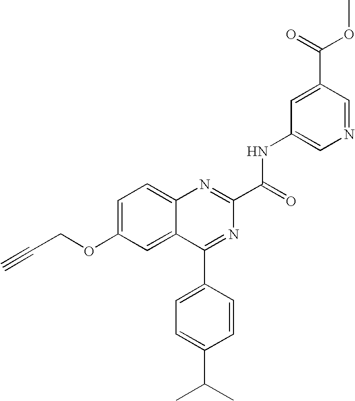

¹H-NMR (400 MHz, DMSO-d₆): 11.21 (s, 1H), 9.32 (d, 1H), 8.93 (t, 1H), 8.87 (d, 1H), 8.26 (d, 1H), 7.97 (d, 2H), 7.86 (dd, 1H), 7.72 (d, 1H), 7.56 (d, 2H), 5.02 (d, 2H), 3.93 (s, 3H), 3.80 (t, 1H), 3.07 (hept, 1H), 1.32 (d, 6H).

MS: 481 (M+1)⁺

### EXAMPLE 94

### 5-{[4-(4-Isopropyl-phenyl)-6-prop-2-ynyloxy-quinazoline-2-carbonyl]-amino}-nicotinic acid isopropyl ester

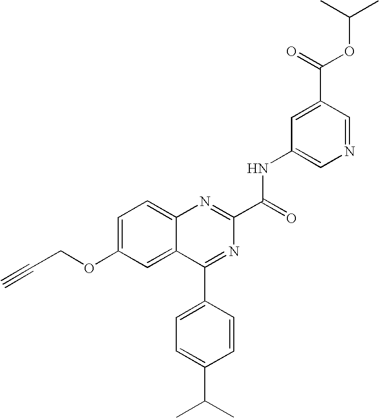

¹H-NMR (400 MHz, CDCl₃): 10.60 (s, 1H), 9.34 (d, 1H), 9.22 (t, 1H), 9.03 (d, 1H), 8.29 (d, 1H), 7.81 (d, 2H), 7.73 (dd, 1H), 7.69 (d, 1H), 7.48 (d, 2H), 5.33 (hept, 1H), 4.80 (d, 2H), 3.07 (hept, 1H), 2.63 (t, 1H), 1.42 (d, 6H), 1.37 (d, 6H).

MS: 509 (M+1)⁺

### EXAMPLE 95

### [4-(4-Isopropyl-phenyl)-6-prop-2-ynyloxy-quinazolin-2-yl]-pyrrol-1-yl-methanone

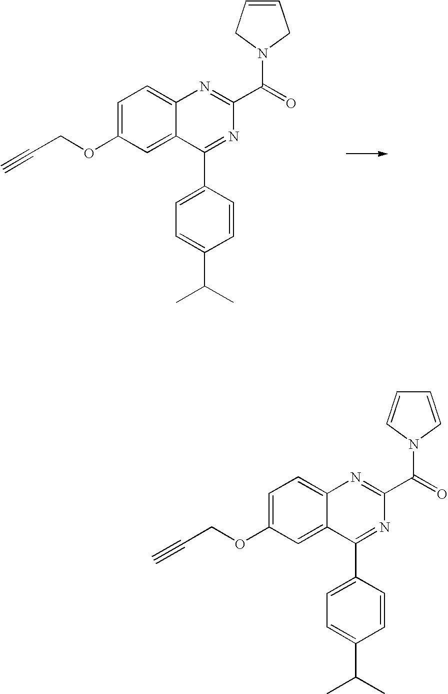

The intermediate (2,5-dihydro-pyrrol-1-yl)-[4-(4-isopropyl-phenyl)-6-prop-2-ynyloxy-quinazolin-2-yl]-methanone is prepared from 4-(4-isopropyl-phenyl)-6-propargyloxy-quinazoline-2-carboxylic acid and commercially available 3-pyrroline using the method described in example 46. A solution of 200 mg (0.50 mmol) of this intermediate and 150 mg (0.65 mmol) DDQ (2,3-dichloro-5,6-dicyano-p-benzoquinone) in 1 ml ethyl acetate is stirred for 18 h at RT. Water is added and the reaction mixture is extracted with ethyl acetate. The solvent is evaporated and the product is purified by flash chromatography using a ethyl acetate/hexane gradient.

¹H-NMR (400 MHz, CDCl₃): 8.22 (m, 1H), 7.86 (d, 2H), 7.72-7.69 (m, 2H), 7.64 (dd, 2H), 7.46 (d, 2H), 6.37 (dd, 1H), 4.83 (d, 2H), 3.06 (hept, 1H), 2.65 (t, 1H), 1.36 (d, 6H).

MS: 396 (M+1)⁺

### EXAMPLE 96

### 4-(4-Isopropyl-phenyl)-6-prop-2-ynyloxy-quinazoline-2-carboxylic acid (5-methyl-1H-pyrazol-3-yl)-amide

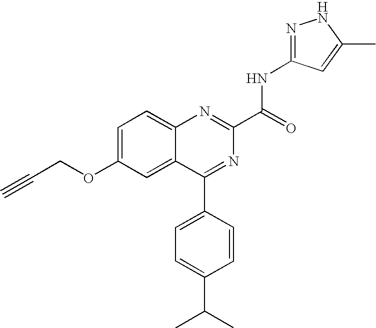

¹H-NMR (400 MHz, CDCl₃): 10.64 (s, 1H), 8.24 (d, 1H), 7.80 (d, 2H), 7.65-7.64 (m, 1H), 7.44 (d, 2H), 6.69 (broad, 1H), 4.78 (d, 2H), 3.05 hept, 1H), 2.62 (t, 1H), 2.34 (s, 3H), 1.35 (d, 6H).

MS: 426 (M+1)⁺

### EXAMPLE 97

### (2-{[4-(4-Isopropyl-phenyl)-6-prop-2-ynyloxy-quinazoline-2-carbonyl]-amino}-thiazol-4-yl)-acetic acid ethyl ester

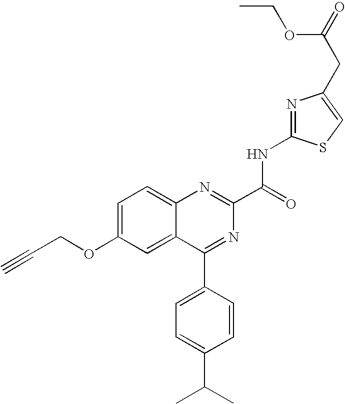

¹H-NMR (400 MHz, CDCl₃): 8.28 (d, 1H), 7.86 (d, 2H), 7.72-7.70 (m, 2H), 7.46 (d, 2H), 6.94 (s, 1H), 4.80 (d, 2H), 4.22 (q, 2H), 3.78 (s, 2H), 3.06 (hept, 1H), 2.62 (t, 1H), 1.36 (d, 6H), 1.29 (t, 3H).

MS: 515 (M+1)⁺

### EXAMPLE 98

### 4-(4-Isopropyl-phenyl)-6-prop-2-ynyloxy-quinazoline-2-carboxylic acid naphthalen-1-ylamide

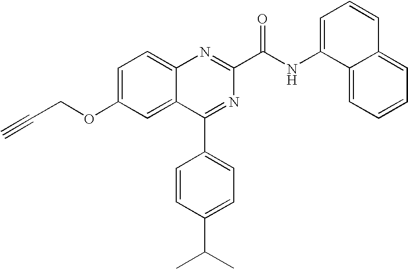

¹H-NMR (400 MHz, CDCl₃): 11.04 (s, 1H), 8.53 (d, 1H), 8.38 (d, 1H), 8.10 (d, 1H), 7.94-7.90 (m, 3H), 7.73-7.71 (m, 3H), 7.61-7.50 (m, 5H), 4.82 (d, 2H), 3.09 (hept, 1H), 2.64 (t, 1H), 1.39 (d, 6H).

MS: 472 (M+1)⁺

### EXAMPLE 99

### 4-(4-Isopropyl-phenyl)-6-prop-2-ynyloxy-quinazoline-2-carboxylic acid isoquinolin-8-ylamide

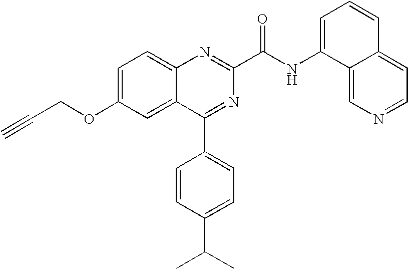

¹H-NMR (400 MHz, CDCl₃): 11.28 (s, 1H), 9.64 (s, 1H), 8.70 (d, 1H), 8.61 (d, 1H), 8.37 (d, 1H), 7.91 (d, 2H), 7.81 (t, 1H), 7.75-7.72 (m, 3H), 7.69 (d, 1H), 7.53 (d, 2H), 4.82 (d, 2H), 3.09 (hept, 1H), 2.65 (t, 1H), 1.39 (d, 6H).

MS: 473 (M+1)⁺

### EXAMPLE 100

### 4-(4-Isopropyl-phenyl)-6-prop-2-ynyloxy-quinazoline-2-carboxylic acid phthalazin-5-ylamide

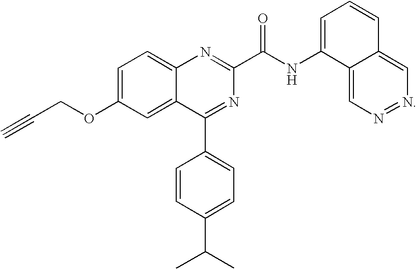

¹H-NMR (400 MHz, CDCl₃): 11.38 (s, 1H), 10.31 (s, 1H), 9.74 (s, 1H), 8.93 (d, 1H), 8.33 (d, 1H), 8.29 (t, 1H), 8.09 (d, 1H), 7.85 (d, 2H), 7.72-7.68 (m, 2H), 7.42 (d, 2H), 4.80 (s, 2H), 3.00 (hept, 1H), 2.65 (s, 1H), 1.32 (d, 6H).

MS: 474 (M+1)⁺

### EXAMPLE 101

### 4-(4-Isopropyl-phenyl)-6-prop-2-ynyloxy-quinazoline-2-carboxylic acid quinolin-5-ylamide

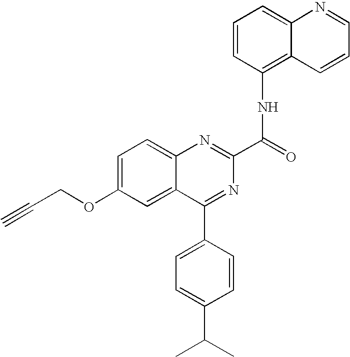

¹H-NMR (400 MHz, CDCl₃): 10.90 (s, 1H), 8.97 (dd, 1H), 8.44 (d, 2H), 8.34 (d, 1H), 8.02 (d, 1H), 7.90 (d, 2H), 7.81 (t, 1H), 7.73-7.70 (m, 2H), 7.51-7.48 (m, 3H), 4.81 (d, 2H), 3.08 (hept, 1H), 2.64 (t, 1H), 1.38 (d, 6H).

MS: 473 (M+1)⁺

### EXAMPLE 102

### 4-(4-Isopropyl-phenyl)-6-prop-2-ynyloxy-quinazoline-2-carboxylic acid quinolin-8-ylamide

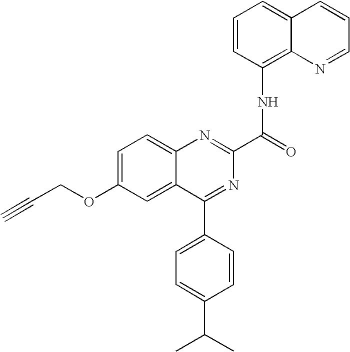

¹H-NMR (400 MHz, CDCl₃): 12.62 (s, 1H), 9.11 (dd, 1H), 8.95 (dd, 1H), 8.38 (d, 1H), 8.21 (dd, 1H), 8.04 (d, 2H), 7.77 (d, 1H), 7.69 (dd, 1H), 7.65 (t, 1H), 7.59 (dd, 1H), 7.52-7.49 (m, 3H), 4.81 (d, 2H), 3.08 (hept, 1H), 2.64 (t, 1H), 1.38 (d, 6H).

MS: 473 (M+1)⁺

### EXAMPLE 103

### 4-(4-Isopropyl-phenyl)-6-prop-2-ynyloxy-quinazoline-2-carboxylic acid isoquinolin-4-ylamide

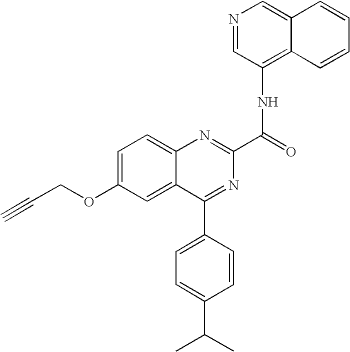

¹H-NMR (400 MHz, CDCl₃): 10.52 (s, 1H), 9.12 (d, 1H), 9.03 (d, 1H), 8.32 (d, 1H), 8.09 (d, 1H), 7.90 (dd, 1H), 7.86 (d, 2H), 7.71 (dd, 1H), 7.68 (d, 1H), 7.66 (ddd, 1H), 7.57 (ddd, 1H), 7.50 (d, 2H), 4.80 (d, 2H), 3.08 (hept, 1H), 2.63 (t, 1H), 1.38 (d, 6H).

MS: 473 (M+1)⁺

### EXAMPLE 104

### 4-(4-Isopropyl-phenyl)-6-prop-2-ynyloxy-quinazoline-2-carboxylic acid (5-acetyl-quinolin-8-yl)-amide

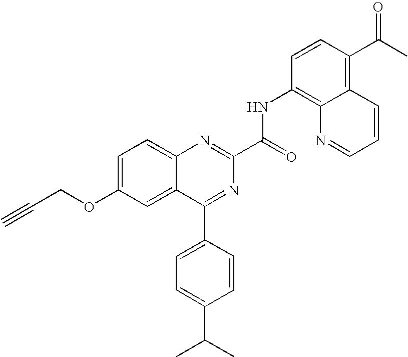

¹H-NMR (400 MHz, CDCl₃): 12.92 (s, 1H), 9.55 (dd, 1H), 9.13 (d, 1H); 8.98 (dd, 1H), 8.40 (d, 1H), 8.29 (d, 1H), 8.05 (d, 2H), 7.79 (d, 1H), 7.72 (dd, 1H), 7.64 (dd, 1H), 7.53 (d, 2H), 4.83 (d, 2H), 3.10 (hept, 1H), 2.78 (s, 3H), 2.66 (t, 1H), 1.40 (d, 6H).

MS: 515 (M+1)⁺

### EXAMPLE 105

### 4-(4-Isopropyl-phenyl)-6-prop-2-ynyloxy-quinazoline-2-carboxylic acid (3-bromo-6-methoxy-quinolin-8-yl)-amide

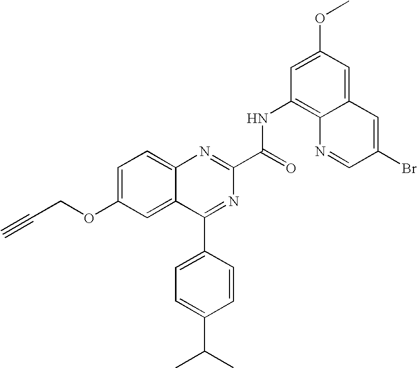

¹H-NMR (400 MHz, CDCl₃): 12.43 (s, 1H), 8.87 (d, 1H), 8.75 (d, 1H), 8.39 (d, 1H), 8.22 (d, 1H), 8.02 (d, 2H), 7.78 (d, 1H), 7.71 (dd, 1H), 7.52 (d, 2H), 6.79 (d, 1H), 4.83 (d, 2H), 3.98 (s, 3H), 3.11 (hept, 1H), 2.65 (t, 1H), 1.40 (d, 6H).

MS: 581/583 (M+1)⁺ (isotope pattern for 1 Br)

### EXAMPLE 106

### 4-(4-Isopropyl-phenyl)-6-prop-2-ynyloxy-quinazoline-2-carboxylic acid quinolin-2-ylamide

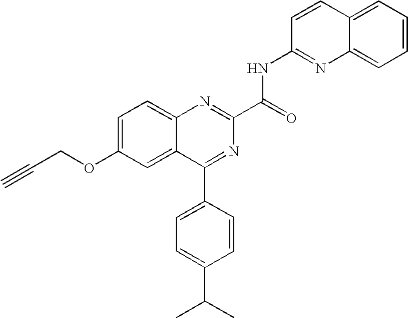

¹H-NMR (400 MHz, CDCl₃): 11.01 (broad, 1H). 8.82 (d, 1H), 8.34 (d. 1H), 8.30 (d, 1H). 7.96 (d, 1H), 7.90 (d, 2H), 7.84 (d, 1H), 7.74-7.70 (m, 3H), 7.51-7.48 (m, 3H), 4.82 (d, 2H), 3.09 (hept, 1H), 2.64 (t, 1H), 1.39 (d, 6H).

MS: 473 (M+1)⁺

### EXAMPLE 107

### 4-(4-Isopropyl-phenyl)-6-prop-2-ynyloxy-quinazoline-2-carboxylic acid quinolin-6-ylamide

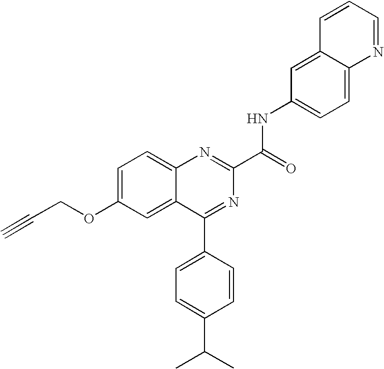

¹H-NMR (400 MHz, CDCl₃): 10.51 (s, 1H), 8.85 (dd, 1H), 8.71 (d, 1H), 8.31 (dd, 1H), 8.23 (dd, 1H), 8.13 (d, 1H), 7.88 (dd, 1H), 7.86 (d, 2H), 7.70 (dd, 1H), 7.67 (d, 1H), 7.50 (d, 2H), 7.42 (dd, 1H), 4.79 (d, 2H), 3.08 (hept, 1H), 2.62 (t, 1H), 1.38 (d, 6H).

MS: 473 (M+1)⁺

### EXAMPLE 108

### 4-(4-Isopropyl-phenyl)-6-prop-2-ynyloxy-quinazoline-2-carboxylic acid (2-methyl-quinolin-6-yl)-amide

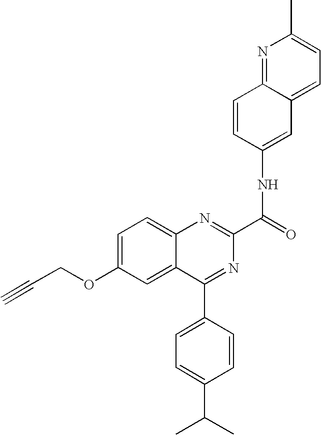

¹H-NMR (400 MHz, CDCl₃): 10.48 (s, 1H), 8.67 (d, 1H), 8.31 (d, 1H), 8.12 (d, 1H), 8.05 (d, 1H), 7.86 (d, 2H), 7.83 (dd, 1H), 7.69 (dd, 1H), 7.67 (d, 1H), 7.49 (d, 2H), 7.30 (d, 1H), 4.79 (d, 2H), 3.08 (hept, 1H), 2.75 (s, 3H), 2.62 (t, 1H), 1.38 (d, 6H).

MS: 487 (M+1)⁺

### EXAMPLE 109

### (6-{[4-(4-Isopropyl-phenyl)-6-prop-2-ynyloxy-quinazoline-2-carbonyl]-amino}-quinolin-8-yloxy)-acetic acid ethyl ester

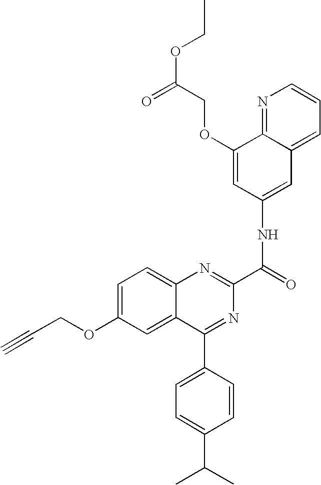

¹H-NMR (400 MHz, CDCl₃): 10.41 (s, 1H), 8.88 (dd, 1H), 8.32 (d, 1H), 8.15 (dd, 1H), 7.97 (d, 1H), 7.86 (d, 2H), 7.71 (dd, 1H), 7.66 (dd, 1H), 7.50 (d, 2H), 7.43 (dd, 1H), 5.04 (s, 2H), 4.80 (d, 2H), 4.30 (q, 2H), 3.08 (hept, 1H), 2.62 (t, 1H), 1.38 (d, 6H), 1.29 (t, 1H).

MS: 575 (M+1)⁺

### EXAMPLE 110

### 4-(4-Isopropyl-phenyl)-6-prop-2-ynyloxy-quinazoline-2-carboxylic acid (1H-benzoimidazol-4-yl)-amide

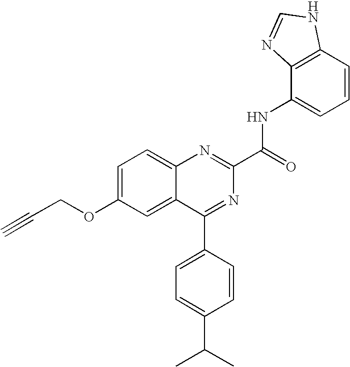

¹H-NMR (400 MHz, DMSO-d₆): 12.68 (s, 1H), 11.19 (s, 1H), 8.32-8.27 (m, 3H), 7.95 (d, 2H), 7.87 (dd, 1H), 7.72 (d, 1H), 7.59 (d, 2H), 7.34 (d, 1H), 7.27 (t, 1H), 5.02 (d, 2H), 3.81 (t, 1H), 3.08 (hept, 1H), 1.34 (d, 6H).

MS: 575 (M+1)⁺

### EXAMPLE 111

### 4-(4-Isopropyl-phenyl)-6-prop-2-ynyloxy-quinazoline-2-carboxylic acid benzothiazol-2-ylamide

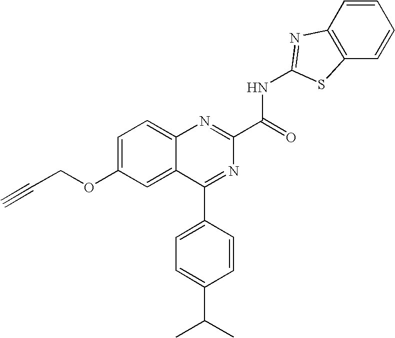

¹H-NMR (400 MHz, CDCl₃): 11.56 (broad, 1H), 8.30 (d, 1H), 7.90-7.84 (m, 4H), 7.73 (dd, 1H), 7.71 (d, 1H), 7.50-7.46 (m, 3H), 7.35 (td, 1H), 4.81 (d, 2H), 3.07 (hept, 1H), 2.63 (t, 1H), 1.37 (d, 6H).

MS: 479 (M+1)⁺

### EXAMPLE 112

### 4-(4-Isopropyl-phenyl)-6-prop-2-ynyloxy-quinazoline-2-carboxylic acid (benzo[1,3]dioxol-5-ylmethyl)-amide

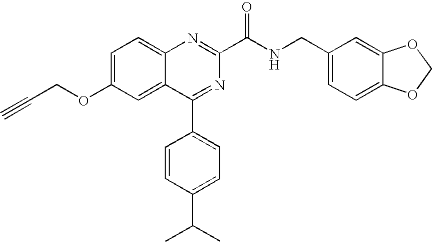

¹H-NMR (400 MHz, CDCl₃): 8.59 (broad, 1H), 8.29 (d, 1H), 7.78 (d, 2H), 7.68-7.63 (m, 2H), 7.44 (d, 2H), 6.92 (s, 1H), 6.87 (d, 1H), 6.77 (d, 1H), 5.94 (s, 2H), 4.78 (d, 2H), 4.68 (m, 2H), 3.04 (hept, 1H), 2.61 (t, 1H), 1.34 (d, 6H).

MS: 480 (M+1)⁺

### EXAMPLE 113

### 4-(4-Isopropyl-phenyl)-6-prop-2-ynyloxy-quinazoline-2-carboxylic acid (thiophen-2-ylmethyl)-amide

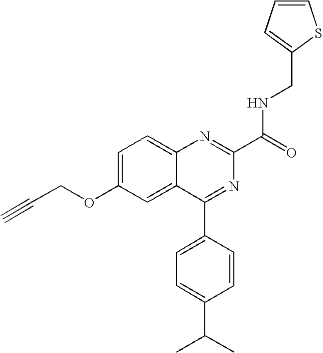

¹H-NMR (400 MHz, CDCl₃): 8.65 (t, 1H), 8.24 (d, 1H), 7.77 (d, 2H), 7.66 (dd, 1H), 7.63 (d, 1H), 7.43 (d, 2H), 7.24 (dd, 1H), 7.09 (dd, 1H), 6.96 (dd, 1H), 4.93 (d, 2H), 4.77 (d, 2H), 3.03 (hept, 1H), 2.60 (t, 1H), 1.34 (d, 6H).

MS: 442 (M+1)⁺

### EXAMPLE 114

### 4-(4-Isopropyl-phenyl)-6-prop-2-ynyloxy-quinazoline-2-carboxylic acid 3-methoxy-phenyl ester

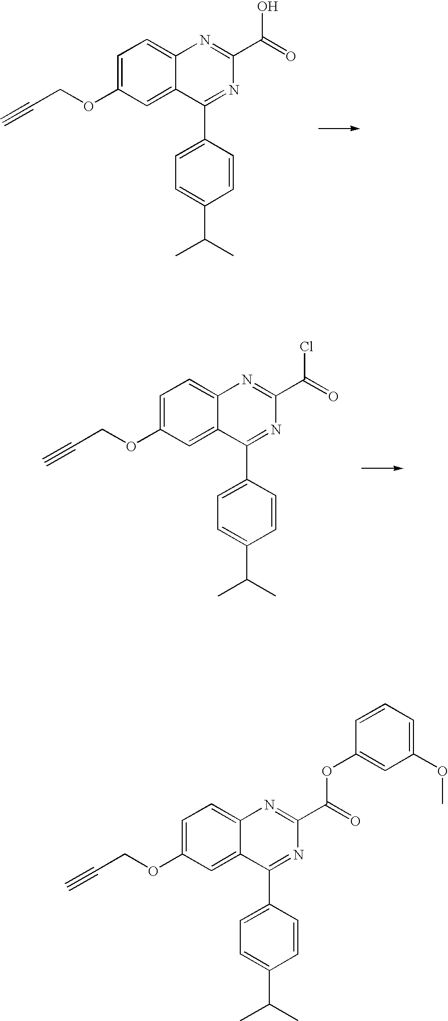

To a solution of 1.00 g (2.9 mmol)) 4-(4-isopropyl-phenyl)-6-propargyloxy-quinazoline-2-carboxylic acid in THF are added slowly 740 μl (8.7 mmol) oxalylic chloride. After 3 h at RT the solvent is evaporated and in order to remove residual oxalylic chloride, toluene is added and evaporated. A portion of the so prepared acid chloride [200 mg (0.55 mmol)] are dissolved in 0.5 ml dichloromethane before 94 μl (0.55 mmol) N-ethyldiisopropylamine and 68 mg (0.55 mmol) 3-methoxyphenol are added. After stirring overnight, 0.1 M hydrochloric acid is added and the reaction mixture is extracted with dichloromethane. The crude product is purified by flash chromatography using a ethyl acetate/hexane gradient.

¹H-NMR (400 MHz, CDCl₃): 8.33 (d, 1H), 7.89 (d, 2H), 7.74-7.69 (m, 2H), 7.46 (d, 2H), 7.35 (t, 1H), 6.95 (ddd, 1H), 6.92 (t, 1H), 6.86 (ddd, 1H), 4.82 (d, 2H), 3.84 (s, 3H), 3.06 (hept, 1H), 2.65 (t, 1H), 1.36 (d, 6H).

MS: 453 (M+1)⁺

### EXAMPLE 115

### 1-(4-Isopropyl-phenyl)-7-prop-2-ynyloxy-isoquinoline-3-carboxylic acid ethyl ester

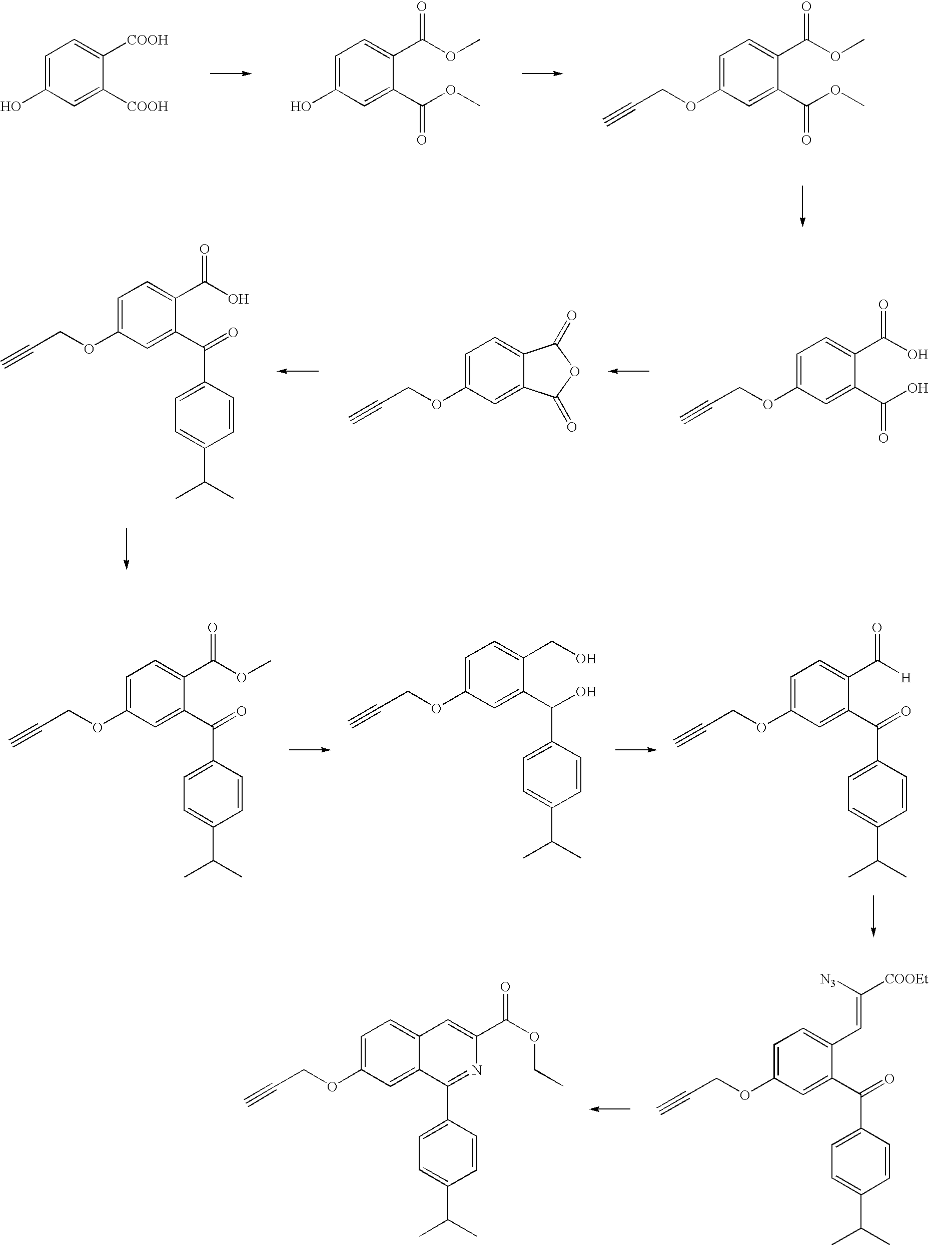

### A) Preparation of 4-hydroxy-phthalic acid dimethyl ester

A solution of 50 g (270 mmol) 4-hydroxy-phthalic acid and 7.4 ml concentrated sulfuric acid in 500 ml methanol is heated under reflux for 11 h. The methanol is evaporated and the residue dissolved in dichloromethane. Upon addition of hexane the dimethyl ester precipitates as white crystals.

Retention time: 1.60 min, MS: 211 (M+1)⁺

### B) Preparation of 4-prop-2-ynyloxy-phthalic acid dimethyl ester

To a solution of the whole amount of the product from above 105 g (1.1 mol) potassium carbonate (150 g, 1.1 mol) and 10 minutes later 43 ml (400 mmol) propargyl bromide (80% in toluene), are added. After stirring for 3 h at RT water is added and the reaction mixture is extracted with MTBE. After evaporation of the solvent the product is obtained that is used in the next step without purification.

Retention time: 2.15 min, MS: 249 (M+1)⁺

### C) Preparation of 4-prop-2-ynyloxy-phthalic acid

The product from above is dissolved in 500 ml methanol and treated with 31 g (780 mmol) sodium hydroxide dissolved in 100 ml water. After stirring overnight at RT the methanol is evaporated and the residues is taken up into water. Upon addition of concentrated hydrochloric acid at 0° C. the free diacid precipitates which is dried in a vacuum oven at 70° C.

Retention time: 1.64 min, MS: 221 (M+1)⁺

### D) Preparation of 5-prop-2-ynyloxy-isobenzofuran-1,3-dione

The diacid from above (50 g, 230 mmol) is heated under reflux in 350 ml acetic anhydride for 24 h. The volatile components are evaporated and the remaining residue is dissolved in toluene and evaporated twice to remove residual acetic acid or anhydride.

A small sample is dissolved in MeOH and reacts to the corresponding mono methyl ester which is detected by HPLC-MS:

Retention time: 1.91 min, MS: 235 (M+1)⁺

### E) Preparation of 2-(4-isopropyl-benzoyl)-4-prop-2-ynyloxy-benzoic acid

A Grignard reagent is prepared in 400 ml THF from 66 g (330 mmol) 4-bromo-isopropylbenzene and 8.1 g Magnesium. Unreacted metallic magnesium is filtered off and the reagent solution is added dropwise at RT to a solution of 55.97 g (280 mmol) 5-prop-2-ynyloxy-isobenzofuran-1,3-dione in 400 ml THF. Cooling is applied to compensate for the exothermic reaction. Fifteen minutes after the end of the addition 500 ml saturated ammonium chloride solution are poured to the reaction mixture and THF is evaporated. The product is extracted with dichloromethane and purified by Flash chromatography using a ethyl acetate/hexane gradient.

¹H-NMR (300 MHz, CDCl₃): 10.52 (broad 1H), 8.04 (d, 1H), 7.65 (d, 2H), 7.25 (d, 2H), 7.07 (dd, 1H), 6.86 (d, 1H), 4.73 (d, 2H), 2.53 (t, 1H), 1.26 (d, 6H).

Retention time: 2.42 min, MS: 323 (M+1)⁺

### F) Preparation of 2-(4-isopropyl-benzoyl)-4-prop-2-ynyloxy-benzoic acid methyl ester

A solution of 8.6 g (27 mmol) 2-(4-isopropyl-benzoyl)-4-prop-2-ynyloxy-benzoic acid and 710 μl sulfuric acid in 50 ml methanol is heated at 60° C. for 16 h. After evaporation of the solvent, water is added and the product is extracted with dichloromethane.

HPLC retention time: 2.62 min, MS: 337 (M+1)⁺

### G) Preparation of (2-hydroxymethyl-5-prop-2-ynyloxy-phenyl)-(4-isopropyl-phenyl)-methanol

A solution of 9.14 g (27 mmol) 2-(4-isopropyl-benzoyl)-4-prop-2-ynyloxy-benzoic acid methyl ester in 60 ml THF is treated with 82 ml (82 mmol) of a solution of LiAlH₄ (1M in THF). Cooling is applied to compensate for the exothermic reaction. Ten minutes after the end of the addition 3.37 ml water are dropped very slowly to the reaction mixture followed by 2.45 ml 20% NaOH. After addition of further 9.14 ml water and stirring for 1 h at RT a white powder can be filtered off. Water is added to the filtrate and the product is extracted with dichloromethane.

HPLC retention time: 2.36 min, MS: 293 (M−17)⁺

### H) Preparation of 2-(4-isopropyl-benzoyl)-4-prop-2-ynyloxy-benzaldehyde

To a solution of 14 g (63 mmol) pyridinium chloro chromate in 60 ml dichloromethane are added 6.5 g (21 mmol) 2-hydroxymethyl-5-prop-2-ynyloxy-phenyl)-(4-isopropyl-phenyl)-methanol dissolved in 20 ml of the same solvent. After 30 minutes stirring at RT the reaction mixture is poured onto water and extracted with dichloromethane. The product is purified by flash chromatography using a ethyl acetate/hexane gradient followed by recrystallization from ethanol.

¹H-NMR (400 MHz, CDCl₃): 9.89 (s, 1H), 8.02 (d, 1H), 7.75 (d, 2H), 7.31 (d, 2H), 7.22 (dd, 1H), 7.03 (d, 1H), 4.79 (d, 2H), 2.99 (hept, 1H), 2.58 (t, 1H), 1.28 (d, 6H).

Retention time: 2.56 min, MS: 307 (M+1)⁺

### I) Preparation of (Z)-2-Azido-3-[2-(4-isopropyl-benzoyl)-4-prop-2-ynyloxy-phenyl]-acrylic acid ethyl ester

With 10 ml ethanol 4.8 ml of a sodium ethylate solution (21% in ethanol) is diluted. To this ethoxide solution is added at 0° C. 1.00 g (3.3 mmol) 2-(4-isopropyl-benzoyl)-4-prop-2-ynyloxy-benzaldehyde dissolved in 7.8 ml (13 mmol) of a 25% solution of ethyl-azidoacetate in ethanol. After 1 h the temperature is allowed to reach RT and stirring is continued overnight. The crude product is obtained after addition of water and extraction with dichloromethane.

¹H-NMR (400 MHz, CDCl₃): 8.20 (d, 1H), 7.76 (d, 2H), 7.31 (d, 2H), 7.15 (dd, 1H), 7.00 (d, 1H), 4.72 (d, 2H), 4.20 (q, 2H), 2.98 (hept, 1H), 2.53 (t, 1H), 1.27 (d, 6H), 1.22 (t, 3H).

Retention time: 2.87 min, MS: 390 (M−28+1)⁺

### J) Preparation of 1-(4-Isopropyl-phenyl)-7-prop-2-ynyloxy-isoquinoline-3-carboxylic acid ethyl ester

A solution of the crude product from above in 50 ml toluene is treated with 2 ml (11 mmol) triethyl phosphite. After 2 h water is added and the reaction mixture is extracted with dichloromethane. The product is purified by flash chromatography using a ethyl acetate/hexane gradient.

¹H-NMR (400 MHz, CDCl₃): 8.47 (s, 1H), 7.94 (d, 1H), 7.70 (d, 2H), 7.45 (dd, 1H), 7.64 (d, 1H), 7.37 (d, 2H), 4.72 (d, 2H), 4.50 (q, 2H), 3.00 (hept, 1H). 2.57 (t, 1H), 1.45 (t, 3H), 1.31 (d, 6H).

MS: 374 (M+1)⁺

### EXAMPLE 116

### 1-(4-Isopropyl-phenyl)-7-prop-2-ynyloxy-isoquinoline-3-carboxylic acid 1,2-dimethyl-propyl ester

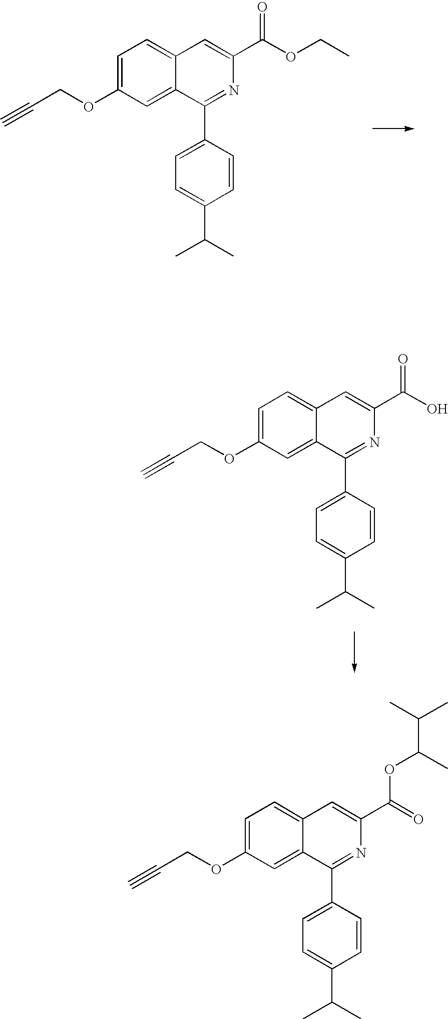

### A) 1-(4-Isopropyl-phenyl)-7-prop-2-ynyloxy-isoquinoline-3-carboxylic acid

To a solution of 1.5 g (4.0 mmol) 1-(4-isopropyl-phenyl)-7-prop-2-ynyloxy-isoquinoline-3-carboxylic acid ethyl ester in 10 ml ethanol are added 4 ml 2 M aqueous sodium hydroxide solution. After 1 h stirring at RT the reaction mixture is set acidic with 1 M hydrochloric acid and extracted with dichloromethane.

¹H-NMR (400 MHz, CDCl₃): 8.58 (s, 1H), 8.03 (d, 1H), 7.70-7.68 (m, 3H), 7.53 (dd, 1H), 7.45 (d, 2H), 4.77 (d, 2H), 3.06 (hept, 1H), 2.59 (t, 1H), 1.37 (d, 6H).

MS: 346 (M+1)⁺

### B) 1-(4-Isopropyl-phenyl)-7-prop-2-ynyloxy-isoquinoline-3-carboxylic acid 1,2-dimethyl-propyl ester

To a solution of 50 mg (0.14 mmol) of the acid from above in 1 ml dichloromethane are added 19 μl (0.22 mmol) oxalyl chloride. After 2 h stirring at RT 47 μl (0.43 mmol) 3-methyl-2-butanol are added. After completion of the reaction within 1 h, 1 ml DMSO is added and the reaction mixture is directly purified by preparative reversed phase HPLC.

¹H-NMR (400 MHz, CDCl₃): 8.40 (s, 1H), 7.94 (d, 1H), 7.73 (d, 2H), 7.67 (d, 1H), 7.45 (dd, 1H), 7.37 (d, 2H), 5.09 (quint, 1H), 4.74 (d, 2H), 3.00 (hept, 1H), 2.58 (t, 1H), 2.01 (oct, 1H), 1.36 (d, 3H), 1.32 (d, 6H), 1.05 (d, 3H), 1.03 (d, 3H).

MS: 416 (M+1)⁺

The compounds of the following examples are prepared in an analogous manner using the appropriate starting materials:

### EXAMPLE 117

### 1-(4-Isopropyl-phenyl)-7-prop-2-ynyloxy-isoquinoline-3-carboxylic acid isobutyl ester

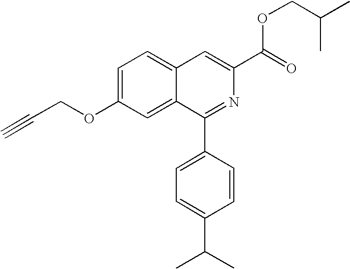

¹H-NMR (400 MHz, CDCl₃): 8.44 (s, 1H), 7.95 (d, 1H), 7.72 (d, 2H), 7.67 (d, 1H), 7.46 (dd, 1H), 7.38 (d, 2H), 4.74 (d, 2H), 4.23 (d, 2H), 3.01 (hept, 1H), 2.57 (t, 1H), 2.18 (non, 1H), 1.32 (d, 6H), 1.05 (d, 6H).

MS: 402 (M+1)⁺

### EXAMPLE 118

### 1-(4-Isopropyl-phenyl)-7-prop-2-ynyloxy-isoquinoline-3-carboxylic acid cyclopropylmethyl ester

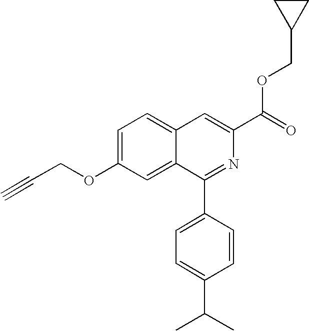

¹H-NMR (400 MHz, DMSO d₆): 8.59 (s, 1H), 8.27 (d, 1H), 7.68 (d, 2H), 7.62-7.59 (m, 2H), 7.47 (d, 2H), 4.92 (d, 2H), 4.20 (d, 2H), 3.73 (t, 1H), 3.03 (hept, 1H), 1.30 (d, 6H), 0.61-0.56 (m, 2H), 0.41-0.37 (m, 2H).

MS: 400 (M+1)⁺

### EXAMPLE 119

### 1-(4-Isopropyl-phenyl)-7-prop-2-ynyloxy-isoquinoline-3-carboxylic acid benzyl ester

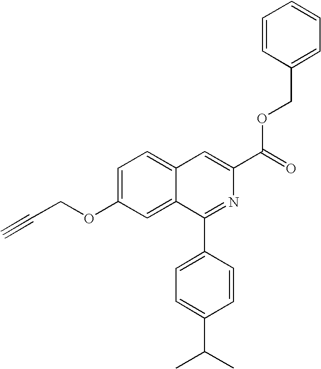

¹H-NMR (400 MHz, CDCl₃): 8.48 (s, 1H), 7.94 (d, 1H), 7.71 (d, 2H), 7.66 (d, 1H), 7.53 (d, 2H), 7.47 (dd, 1H), 7.41-7.34 (m, 5H), 5.50 (s, 2H), 4.74 (d, 2H), 3.01 (hept, 1H), 2.57 (t, 1H), 1.32 (d, 6H).

MS: 436 (M+1)⁺

### EXAMPLE 120

### 1-(4-Isopropyl-phenyl)-7-prop-2-ynyloxy-isoquinoline-3-carboxylic acid 2-methoxy-benzyl ester

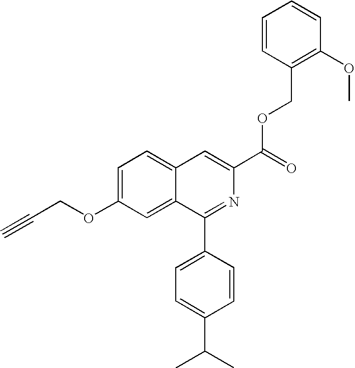

¹H-NMR (400 MHz, CDCl₃): 8.48 (s, 1H), 7.94 (d, 1H), 7.72 (d, 2H), 7.66 (d, 1H), 7.51 (dd, 1H), 7.47 (dd, 1H), 7.38 (d, 2H), 7.32 (ddd, 1H), 6.98 (td, 1H), 6.92 (d, 1H), 5.55 (s, 2H), 4.74 (d, 2H), 3.87 (s, 3H), 3.01 (hept, 1H), 2.57 (t, 1H), 1.32 (d, 6H).

MS: 466 (M+1)⁺

### EXAMPLE 121

### 1-(4-Isopropyl-phenyl)-7-prop-2-ynyloxy-isoquinoline-3-carboxylic acid 3-methoxy-benzyl ester

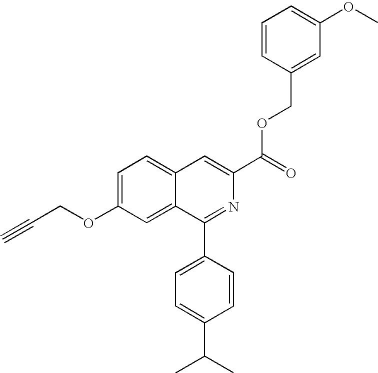

¹H-NMR (400 MHz, CDCl₃): 8.48 (s, 1H), 7.94 (d, 1H), 7.71 (d, 2H), 7.66 (d, 1H), 7.46 (dd, 1H), 7.38 (d, 2H), 7.30 (t, 1H), 7.10-7.08 (m, 2H), 6.89-6.86 (m, 1H), 5.47 (s, 2H), 4.74 (d, 2H), 3.82 (s, 3H), 3.01 (hept, 1H), 2.57 (t, 1H), 1.32 (d, 6H).

MS: 466 (M+1)⁺

### EXAMPLE 122

### 1-(4-Isopropyl-phenyl)-7-prop-2-ynyloxy-isoquinoline-3-carboxylic acid 4-methoxycarbonyl-benzyl ester

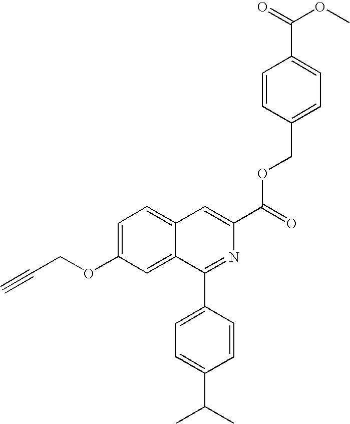

¹H-NMR (400 MHz, CDCl₃): 8.49 (s, 1H), 8.06 (d, 2H), 7.95 (d, 1H), 7.71 (d, 2H), 7.67 (d, 1H), 7.58 (d, 2H), 7.47 (dd, 1H), 7.39 (d, 2H), 5.54 (s, 2H), 4.75 (d, 2H), 3.92 (s, 3H), 3.02 (hept, 1H), 2.58 (t, 1H), 1.33 (d, 6H).

MS: 494 (M+1)⁺

### EXAMPLE 123

### 1-(4-Isopropyl-phenyl)-7-prop-2-ynyloxy-isoquinoline-3-carboxylic acid phenethyl ester

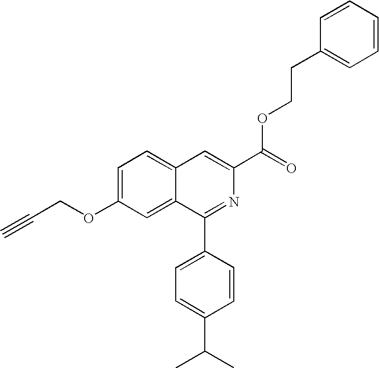

¹H-NMR (400 MHz, CDCl₃): 8.43 (s, 1H), 7.94 (d, 1H), 7.72 (d, 2H), 7.67 (d, 1H), 7.47 (dd, 1H), 7.39 (d, 2H), 7.37-7.30 (m, 4H), 7.26-7.22 (m, 1H), 4.75 (d, 2H), 4.64 (t, 2H), 3.16 (t, 2H), 3.02 (hept, 1H), 2.57 (t, 1H), 1.33 (d, 6H).

MS: 450 (M+1)⁺

### EXAMPLE 124

### 1-(4-Isopropyl-phenyl)-7-prop-2-ynyloxy-isoquinoline-3-carboxylic acid 1-phenyl-ethyl ester

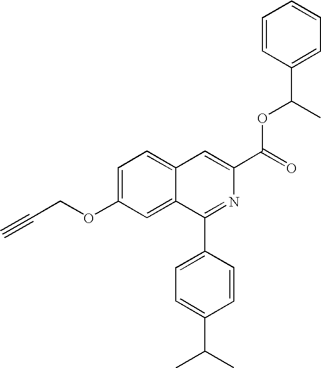

¹H-NMR (400 MHz, CDCl₃): 8.45 (s, 1H), 7.95 (d, 1H), 7.73 (d, 2H), 7.67 (d, 1H), 7.53 (d, 2H), 7.46 (dd, 2H), 7.40-7.36 (m, 4H), 7.30 (tt, 1H), 6.26 (q, 1H), 4.74 (d, 2H), 3.01 (hept, 1H), 2.58 (t, 1H), 1.75 (d, 3H), 1.32 (d, 6H).

MS: 450 (M+1)⁺

### EXAMPLE 125

### 1-(4-Isopropyl-phenyl)-7-prop-2-ynyloxy-isoquinoline-3-carboxylic acid [3-(2-hydroxy-ethanesulfonyl)-phenyl]-amide

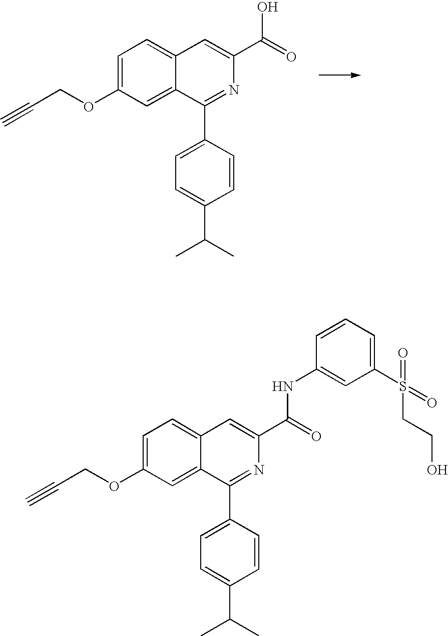

A solution of 75 mg (0.22 mmol) 1-(4-isopropyl-phenyl)-7-prop-2-ynyloxy-isoquinoline-3-carboxylic acid, 52 mg (0.22 mmol) 2-(3-aminophenylsulfonyl)ethanol 93 μl (0.54 mmol) N-ethyl-diisopropylamine and 140 mg (0.33 mmol) BOP in 1 ml THF is stirred for 1 h at RT. DMSO (1 ml) is added and the product is isolated by preparative reversed phase HPLC.

¹H-NMR (400 MHz, DMSO d₆): 10.80 (s, 1H), 8.66 (s, 1H), 8.49 (m, 1H), 8.31 (d, 1H), 8.29-8.27 (m, 1H), 7.90 (d, 2H), 7.69 (d, 1H), 7.67-7.62 (m, 3H), 7.52 (d, 2H), 4.95 (d, 2H), 4.90 (t, 1H), 3.75 (t, 1H), 3.71 (q, 2H), 3.47 (t, 2H), 3.06 (hept, 1H), 1.33 (d, 6H).

MS: 529 (M+1)⁺

### EXAMPLE 126

### [1-(4-Isopropyl-phenyl)-7-prop-2-ynyloxy-isoquinolin-3-yl]-(3-methoxy-phenyl)-methanone

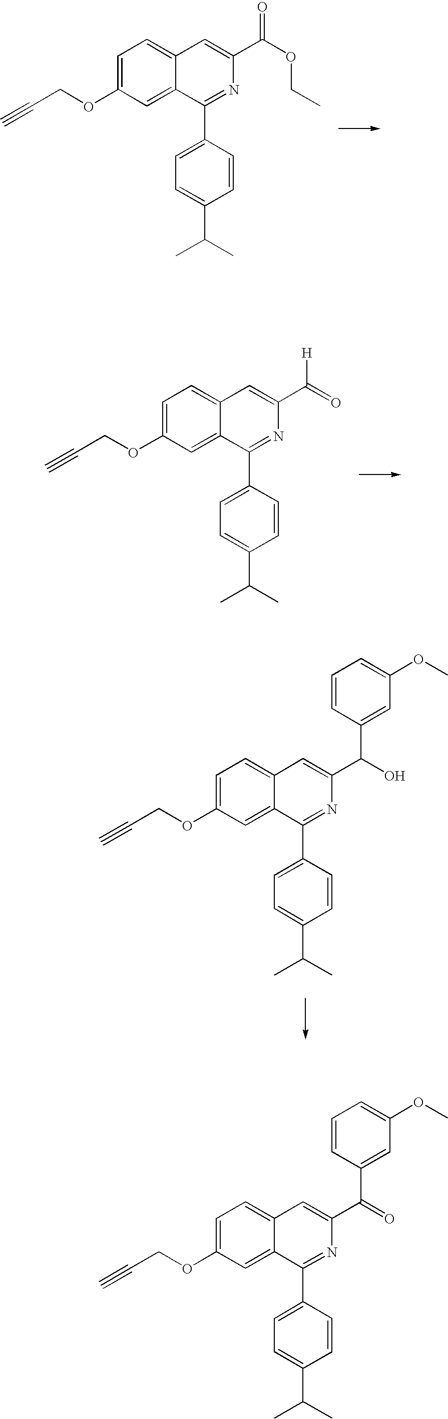

### A) 1-(4-Isopropyl-phenyl)-7-prop-2-ynyloxy-isoquinoline-3-carbaldehyde

To a solution of 1.1 g (2.9 mmol) 1-(4-Isopropyl-phenyl)-7-prop-2-ynyloxy-isoquinoline-3-carboxylic acid ethyl ester in 6 ml THF are added at −78° C. 2.5 ml (2.9 mmol) 1.2 M DIBAH solution in toluene. The reaction mixture is allowed to reach RT and after the addition of water extracted with dichloromethane. Since a mixture of the corresponding alcohol and aldehyde is obtained the crude product dissolved in 5 ml dichloromethane is treated at RT with 1 g (4.6 mmol) pyridinium-chloro-chromate and stirred overnight. Water is added and the reaction mixture is extracted with dichloromethane. The product is purified by flash chromatography using a ethyl acetate/hexane gradient.

### B) [1-(4-Isopropyl-phenyl)-7-prop-2-ynyloxy-isoquinolin-3-yl]-(3-methoxy-phenyl)-methanol

The Grignard reagent prepared from 38 μl (0.3 mmol) 3-bromoanisole and 7.4 mg (0.3 mmol) magnesium in 0.2 ml THF is added at RT to a solution of 50 mg (0.15 mmol) of the aldehyde prepared above in 0.5 ml THF. After 10 minutes saturated ammonium chloride solution is added and the mixture is extracted with dichloromethane. The product is purified by preparative reversed phase HPLC.

### C): [1-(4-Isopropyl-phenyl)-7-prop-2-ynyloxy-isoquinolin-3-yl]-(3-methoxy-phenyl)methanone

A solution of 25 mg (0.057 mmol) of the alcohol prepared above in 0.5 ml dichloromethane is treated with 22 μl (0.057 mmol) Jones reagent. After stirring overnight water is added and the product is extracted with dichloromethane and recrystallized from diethyl ether.

¹H-NMR (400 MHz, CDCl₃): 8.38 (s, 1H), 8.00 (d, 1H), 7.77-7.74 (m, 4H), 7.72 (d, 1H), 7.50 (dd, 1H), 7.41-7.37 (m, 3H), 7.14 (dd, 1H), 4.77 (d, 2H), 3.86 (s, 3H), 3.01 (hept, 1H), 2.59 (t, 1H), 1.32 (d, 6H).

MS: 436 (M+1)⁺

The compound of the following examples are prepared in an analogous manner using the appropriate starting materials:

### EXAMPLE 127

### [1-(4-Isopropyl-phenyl)-7-prop-2-ynyloxy-isoquinolin-3-yl]-(4-methoxy-phenyl)-methanone

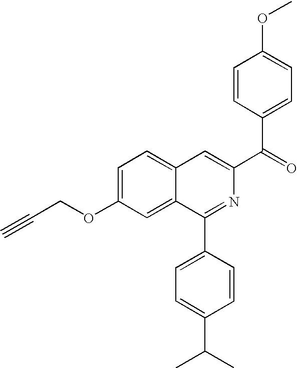

¹H-NMR (400 MHz, CDCl₃): 8.37 (s, 1H), 8.27 (d, 2H), 7.98 (d, 1H), 7.74 (d, 2H), 7.70 (d, 1H), 7.47 (dd, 1H), 7.39 (d, 2H), 6.98 (d, 2H), 4.76 (d, 2H), 3.89 (s, 3H), 3.01 (hept, 1H), 2.59 (t, 1H), 1.33 (d, 6H).

MS: 436 (M+1)⁺

The Agents of the Invention, as defined above, e.g., of formula (I), particularly as exemplified, in free or pharmaceutically acceptable acid addition salt form, exhibit pharmacological activity and are useful as pharmaceuticals, e.g. for therapy, in the treatment of diseases and conditions as hereinafter set forth.

Assay for intracellular free calcium:

A method to determine antagonism at the PcaR consists in measuring the inhibition of intracellular calcium transients stimulated by extracellular calcium.

CCL39 fibroblasts stably transfected with human PcaR are seeded at 40,000 cells/well into 96-well Viewplates and incubated for 24 hours. Medium is then removed and replaced with fresh medium containing 2 μM Fluo-3 AM (Molecular Probes, Leiden, The Netherlands), In routine experiments, cells are incubated at 37° C., 5% CO₂ for 1 h. Afterwards, plates are washed twice with mHBS and wells are refilled with 100 μl mHBS containing the test compounds. Incubation is continued at room temperature for 15 minutes. To record changes of intracellular free calcium, plates are transferred to fluorescence-imaging plate reader (Molecular Devices, Sunnyvale, Calif., USA). A baseline consisting in 5 measurements of 0.4 seconds each (laser excitation 488 nm) is recorded. Cells are then stimulated with calcium (2.5 mM final), and fluorescence changes recorded over a period of 3 minutes.

When measured in the above assays, Agents of the Invention typically have IC₅₀s in the range from about 1000 nM down to about 1 nM or less.

It is now well established that controlled treatment of patients with parathyroid hormone (PTH) and analogues and fragments thereof can have a pronounced anabolic effect on bone formation. Thus compounds which promote PTH release, such as the Agents of the Invention may be used for preventing or treating conditions of bone which are associated with increased calcium depletion or resorption or in which stimulation of bone formation and calcium fixation in the bone is desirable.

Thus in a further aspect the invention includes a method for preventing or treating bone conditions which are associated with increased calcium depletion or resorption or in which stimulation of bone formation and calcium fixation in the bone is desirable in which an effective amount of an Agent of the Invention is administered to a patient in need of such treatment.

In a yet further aspect the invention includes a pharmaceutical composition for preventing or treating bone conditions which are associated with increased calcium depletion or resorption or in which stimulation of bone formation and calcium fixation in the bone is desirable comprising an Agent of the Invention in admixture with a pharmaceutically acceptable excipient, diluent or carrier.

Agents of the Invention are accordingly indicated for preventing or treating all bone conditions which are associated with increased calcium depletion or resorption or in which stimulation of bone formation and calcium fixation in the bone is desirable, e.g. osteoporosis of various genesis (e.g. juvenile, menopausal, post-menopausal, post-traumatic, caused by old age or by corticosteroid therapy or inactivity), fractures, osteopathy, including acute and chronic states associated with skeletal demineralisation, osteo-malacia, periodontal bone loss or bone loss due to arthritis or osteoarthritis or for treating hypoparathyroidism.

Further diseases and disorders which might be prevented or treated include e.g. seizures, stroke, head trauma, spinal cord injury, hypoxia-induced nerve cell damage such as in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, congestive heart failure; hypertension; gut motility disorders such as diarrhoea, and spastic colon and dermatological disorders, e.g. in tissue healing, for example burns, ulcerations and wounds.

The Agents of the Invention are particularly indicated for preventing or treating osteoporosis of various genesis.

For all the above uses, an indicated daily dosage is in the range from about 0.03 to about 300 mg preferably 0.03 to 30, more preferably 0.1 to 10 mg of a compound of the invention. Agents of the Invention may be administered twice a day or up to twice a week.

The Agents of the Invention may be administered in free form or in pharmaceutically acceptable salt form. Such salts may be prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in form of injectable solutions, microemulsions or suspensions, enterally, e.g. orally, for example in the form of tablets or capsules or in a transdermal, nasal or a suppository form.

According to a further embodiment of the invention, the Agents of the Invention may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, bazedoxifene, arzoxifene, FC1271, Tibolone (Livial®), a RANKL antibody, e.g. denosumab, a cathepsin K inhibitor, vitamin D or an analogue thereof or PTH, a PTH fragment or a PTH derivative e.g. PTH (1-84), PTH (1-34), PTH (1-36), PTH (1-38), PTH (1-31)NH₂ or PTS 893.

When the Agents of the Invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

In accordance with the foregoing the present invention further provides:

a) an Agent of the Invention or a pharmaceutically acceptable salt thereof for use as a pharmaceutical;

b) a method for preventing or treating above mentioned disorders and diseases in a subject in need of such treatment, which method comprises administering to said subject an effective amount of an Agent of the Invention or a pharmaceutically acceptable salt thereof;

c) an Agent of the Invention or a pharmaceutically acceptable salt thereof for use in the preparation of a pharmaceutical composition e.g. for use in the method as in b) above.

According to a further embodiment of the invention, the Agents of the Invention may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, TSE-424, FC1271, Tibolone (Livial®), vitamin D or an analogue thereof or PTH, a PTH fragment or a PTH derivative e.g. PTH (1-84), PTH (1-34), PTH (1-36), PTH (1-38), PTH (1-31)NH₂ or PTS 893.

When the Agents of the Invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

1. A compound of formula (I) or a pharmaceutically acceptable salt or prodrug ester thereof: 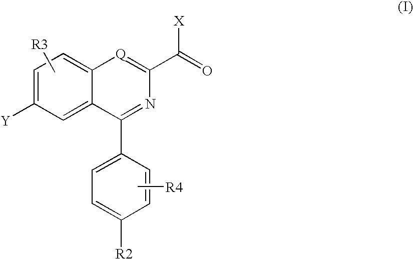
wherein:
Q is CH or N;
R2 is C₁-C₄ alkyl;
Y is selected from the group consisting of: R5-O—, C₁-C₄ alkyl, C₁-C₄ alkenyl, C₁-C₄ alkynyl, R5-NH—;
where R5 is C₁-C₄ alkyl, C₁-C₄ alkenyl, C₁-C₄ alkynyl;
X is selected from the group consisting of aryl, heteroaryl, C₁-C₁₀ alkyl, C₁-C₁₀ alkyloxy, cycloalkyl, heterocycloalkyl, aryl C₁-C₄ alkyl, heteroaryl C₁-C₄ alkyl, cycloalkyl C₁-C₄ alkyl, heterocycloalkyl C₁-C₄ alkyl, arylamino, heteroarylamino, aryl C₁-C₄ alkylamino, heteroaryl C₁-C₄ alkylamino, C₁-C₆ alkylamino, C₁-C₆dialkylamino, aryloxy, heteroaryloxy, aryl C₁-C₄ alkyloxy, heteroaryl C₁-C₄ alkyloxy, cycloalkyl C₁-C₄ alkylamino, heterocycloalkyl C₁-C₄ alkylamino, cycloalkyl C₁-C₄ alkyloxy or heterocycloalkyl C₁-C₄ alkyloxy each of which is optionally substituted once or more;
the optional substituent or substituents on X being independently selected from the group consisting of halo, cyano, trifluoromethyl, nitro, hydroxy, optionally substituted (C₁-C₄ alkyl, C₁-C₄ alkyloxy, amino, sulfanyl, sulfonyl, amino, oxycarbonyl, hydroxyl, sulfinyl, aminosulfonyl, sulfonylamino, carbonyl, carbonyloxy, carbonyl amino, carboxyl, acyl, acylamino, or carbamoyl); the optional substituent or substituents being selected from C₁-C₆ alkyl, C₁-C₆ alkyloxy, carboxyl, hydroxyl, hydroxy C₁-C₄ alkyl; each of which in turn may be optionally substituted by C₁-C₆ alkyloxy, C₁-C₆ alkyl, C₁-C₃ fluorinated alkyl, C₁-C₆ alkyloxy, carboxyl, hydroxyl, hydroxy C₁-C₄ alkyl, halo, cyano, nitro.
R3 and R4 each represent one or more substituents independently selected from: H, halo, C₁-C₄ alkyl, C₁-C₄ alkyloxy, CF₃;
the optional substituent or substituents on R3 or R4 being independently selected from the group consisting of C₁-C₄ alkyl, halo, C₁-C₄ alkyloxy, cyano, sulfanyl, sulfonyl, amino, oxycarbonyl, hydroxyl which may in turn be optionally substituted once or more by C₁-C₄ alkyl, halo, C₁-C₄ alkyloxy, cyano, sulfanyl, sulfonyl, amino, oxycarbonyl or hydroxyl.

2. A compound of formula (I′) or a pharmaceutically acceptable salt or prodrug ester thereof: 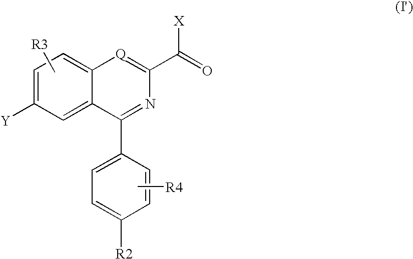
wherein:
Q is CH or N;
R2 is C₁-C₄ alkyl;
Y is selected from the group consisting of: R5-O—, C₁-C₄ alkyl, C₁-C₄ alkenyl, C₁-C₄ alkynyl, R5-NH—;
where R5 is C₁-C₄ alkyl, C₁-C₄ alkenyl, C₁-C₄ alkynyl;
X is selected from the group consisting of aryl, heteroaryl, C₁-C₆ alkyl, cycloalkyl, heterocycloalkyl, aryl C₁-C₄ alkyl, heteroaryl C₁-C₄ alkyl, arylamino, aryl C₁-C₄ alkylamino, heteroaryl C₁-C₄ alkylamino, C₁-C₆ alkylamino, C₁-C₆dialkylamino amino, aryloxy, heteroaryloxy, aryl C1-C4 alkyloxy, or heteroaryl C₁-C₄ alkyloxy, each of which is optionally substituted once or more;
the optional substituent or substituents on X being independently selected from the group consisting of C₁-C₄ alkyl, halo, C₁-C₄ alkyloxy, cyano, trifluoromethyl, hydroxy, amino, nitro, alkyl, lower alkyl substituted (sulfanyl, sulfonyl, amino, oxycarbonyl, hydroxyl, sulfinyl, carbonyl, carboxyl, carbamoyl or aminoacyl);
R3 and R4 each represent one or more substituents independently selected from: H, halo, optionally substituted C₁-C₄ alkyl, optionally substituted C₁-C₄ alkyloxy;
the optional substituent or substituents on R3 or R4 being independently selected from the group consisting of C₁-C₄ alkyl, halo, C₁-C₄ alkyloxy, cyano, sulfanyl, sulfonyl, amino, oxycarbonyl, hydroxyl.

3. A compound of according to claim 1 having the formula (II) or a pharmaceutically acceptable salt or prodrug ester thereof: 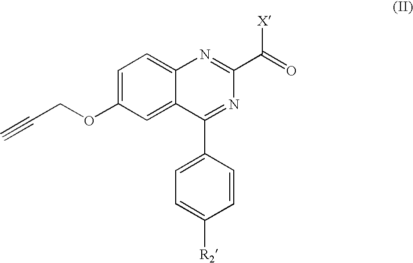
wherein:
X′ is selected from the group consisting of aryl, heteroaryl, cycloalkyl, heterocycloalkyl, —C₁-C₄ alkylaryl, —C₁-C₄ alkylheteroaryl, arylamino, heteroarylamino, aryl C₁-C₄ alkylamino, heteroaryl C₁-C₄ alkylamino, aryloxy, heteroaryloxy, aryl C₁-C₄ alkyloxy, heteroaryl C₁-C₄ alkyloxy, aryl C₁-C₄ alkyl, heteroaryl C₁-C₄ alkyl, C₁-C₆ alkyl, —C₁-C₄ alkylamino or amino, each of which is optionally substituted once or more;
the optional substituent or substituents on X′ being independently selected from the group consisting of halo, cyano, trifluoromethyl, nitro, hydroxy, optionally substituted (C₁-C₄ alkyl, C₁-C₄ alkyloxy, amino, sulfanyl, sulfonyl, amino, oxycarbonyl, hydroxyl, sulfinyl, carbonyl, carboxyl, acyl, acylamino, carbamoyl or aminoacyl); the optional substituent or substituents being selected from C₁-C₆ alkyl, C₁-C₆ alkyloxy, carboxyl, hydroxyl, hydroxy C₁-C₄ alkyl; each of which in turn may be optionally substituted by C₁-C₆ alkyloxy, C₁-C₆ alkyl, C₁-C₆ alkyloxy, carboxyl, hydroxyl, hydroxy C₁-C₄ alkyl, halo, cyano, nitro.
R₂′ is C₁-C₄ alkyl.

4. A compound of according to claim 1 having the formula (II) or a pharmaceutically acceptable salt or prodrug ester thereof: 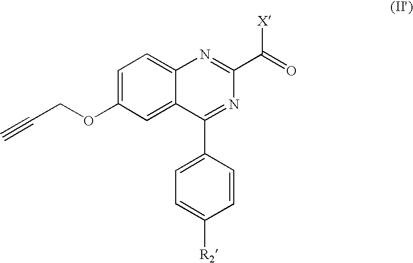
wherein:
X′ is selected from the group consisting of aryl, heteroaryl, cycloalkyl, heterocycloalkyl, C₁-C₄ alkylaryl, C₁-C₄ alkylheteroaryl, C₁-C₄ alkyl, C₁-C₄ alkylamino or amino, each of which is optionally substituted once or more;
the optional substituent or substituents on X′ being independently selected from the group consisting of C₁-C₄ alkyl, halo, C₁-C₄ alkyloxy, cyano, trifluoromethyl, hydroxy, amino, nitro, alkyl, lower alkyl substituted (sulfanyl, sulfonyl, amino, oxycarbonyl, hydroxyl, sulfinyl, carbonyl, carboxyl, carbamoyl or aminoacyl);
R₂′ is C₁-C₄ alkyl.

5. A compound according to claim 3 wherein R₂′ is isopropyl, t-butyl or cyclopropyl.

6. A compound according to claim 2 wherein X′ is optionally substituted (aryl, heteroaryl, heterocycloalkyl, arylamino, heteroarylamino, aryl C₁-C₄ alkylamino, heteroaryl C₁-C₄ alkylamino, aryloxy, heteroaryloxy, C₁-C₆ alkyloxy, aryl C₁-C₄ alkyloxy, heteroaryl C₁-C₄ alkyloxy).

7. A compound according to claim 1 selected from the following:
(4-tert-Butyl-phenyl)-[4-(4-isopropyl-phenyl)-6-propargyloxy-quinazolin-2-yl]-methanone;
[4-(4-Isopropyl-phenyl)-6-propargyloxy-quinazolin-2-yl]-phenyl-methanone;
(2-Methoxy-phenyl)-[4-(4-Isopropyl-phenyl)-6-propargyloxy-quinazolin-2-yl]-methanone;
(3-Methoxy-phenyl)-[4-(4-Isopropyl-phenyl)-6-propargyloxy-quinazolin-2-yl]-methanone;
(4-Methoxy-phenyl)-[4-(4-Isopropyl-phenyl)-6-propargyloxy-quinazolin-2-yl]-methanone;
(4-Fluoro-phenyl)-[4-(4-isopropyl-phenyl)-6-propargyloxy-quinazolin-2-yl]-methanone;
(3-Fluoro-phenyl)-[4-(4-isopropyl-phenyl)-6-propargyloxy-quinazolin-2-yl]-methanone;
(3-Chloro-phenyl)-[4-(4-isopropyl-phenyl)-6-propargyloxy-quinazolin-2-yl]-methanone;
(4-Chloro-phenyl)-[4-(4-isopropyl-phenyl)-6-propargyloxy-quinazolin-2-yl]-methanone;
(4-Fluoro-phenyl)-[4-(4-tert.butyl-phenyl)-6-propargyloxy-quinazolin-2-yl]-methanone;
(3-Fluoro-phenyl)-[4-(4-tert.butyl-phenyl)-6-propargyloxy-quinazolin-2-yl]-methanone;
(3-Bromo-phenyl)-[4-(4-isopropyl-phenyl)-6-propargyloxy-quinazolin-2-yl]-methanone;
(4-Bromo-phenyl)-[4-(4-isopropyl-phenyl)-6-propargyloxy-quinazolin-2-yl]-methanone;
(4-Methyl-phenyl)-[4-(4-isopropyl-phenyl)-6-propargyloxy-quinazolin-2-yl]-methanone;
(4-Isopropyl-phenyl)-[4-(4-isopropyl-phenyl)-6-propargyloxy-quinazolin-2-yl]-methanone;
(4-Ethyl-phenyl)-[4-(4-isopropyl-phenyl)-6-propargyloxy-quinazolin-2-yl]-methanone;
(4-Propyl-phenyl)-[4-(4-isopropyl-phenyl)-6-propargyloxy-quinazolin-2-yl]-methanone;
(4-Cyano-phenyl)-[4-(4-isopropyl-phenyl)-6-propargyloxy-quinazolin-2-yl]-methanone;
(4-Methylthio-phenyl)-[4-(4-isopropyl-phenyl)-6-propargyloxy-quinazolin-2-yl]-methanone;
(4-Methansulfonyl-phenyl)-[4-(4-isopropyl-phenyl)-6-propargyloxy-quinazolin-2-yl]-methanone;
(4-Dimethylamino-phenyl)-[4-(4-isopropyl-phenyl)-6-propargyloxy-quinazolin-2-yl]-methanone;
(4-Ethoxy-phenyl)-[4-(4-isopropyl-phenyl)-6-propargyloxy-quinazolin-2-yl]-methanone;
4-[4-(4-Isopropyl-phenyl)-6-propargyloxy-quinazoline-2-carbonyl]-benzoic acid methyl ester;
(4-Dimethylamino-phenyl)-[4-(4-tert.butyl-phenyl)-6-propargyloxy-quinazolin-2-yl]-methanone;
(4-Dimethylamino-phenyl)-[4-(4-cyclopropyl-phenyl)-6-propargyloxy-quinazolin-2-yl]-methanone;
4-[4-(4-Isopropyl-phenyl)-6-propargyloxy-quinazoline-2-carbonyl]-benzoic acid ethyl ester;
(4-Methoxy-phenyl)-[4-(4-tert.butyl-phenyl)-6-propargyloxy-quinazolin-2-yl]-methanone;
(4-Ethoxy-phenyl)-[4-(4-cyclopropyl-phenyl)-6-propargyloxy-quinazolin-2-yl]-methanone;
(3-Ethoxy-4-methoxy-phenyl)-[4-(4-isopropyl-phenyl)-6-propargyloxy-quinazolin-2-yl]-methanone;
(4-tert.Butyloxy-phenyl)-[4-(4-isopropyl-phenyl)-6-propargyloxy-quinazolin-2-yl]-methanone;
(4-Hydroxy)-[4-(4-isopropyl-phenyl)-6-propargyloxy-quinazolin-2-yl]-methanone;
(4-Butyloxy)-[4-(4-isopropyl-phenyl)-6-propargyloxy-quinazolin-2-yl]-methanone;
Furan-2-yl-[4-(4-isopropyl-phenyl)-6-propargyloxy-quinazolin-2-yl]-methanone;
Furan-3-yl-[4-(4-tert.butyl-phenyl)-6-propargyloxy-quinazolin-2-yl]-methanone;
Furan-3-yl-[4-(4-isopropyl-phenyl)-6-propargyloxy-quinazolin-2-yl]-methanone;
Thiophen-2-yl-[4-(4-isopropyl-phenyl)-6-propargyloxy-quinazolin-2-yl]-methanone;
(3-Methyl-thiophen-2-yl-[4-(4-isopropyl-phenyl)-6-propargyloxy-quinazolin-2-yl]-methanone;
Benz[b]thiophen-2-yl-[4-(4-isopropyl-phenyl)-6-propargyloxy-quinazolin-2-yl]-methanone;
Thiophen-3-yl-[4-(4-isopropyl-phenyl)-6-propargyloxy-quinazolin-2-yl]-methanone;
(1-Methyl-1H-pyrrol)-2-yl-[4-(4-isopropyl-phenyl)-6-propargyloxy-quinazolin-2-yl]-methanone;
4-(4-Isopropyl-phenyl)-6-propargyloxy-quinazoline-2-carboxylic acid ethyl ester;
[4-(4-isopropyl-phenyl)-6-propargyloxy-quinazolin-2-yl]-pyridine-3-yl-methanone;
[4-(4-Isopropyl-phenyl)-6-propargyloxy-quinazolin-2-yl]-naphthalen-1-yl-methanone;
[4-(4-Isopropyl-phenyl)-6-propargyloxy-naphathalen-2-yl]-methanone;
Benzothiazol-2-yl-[4-(4-isopropyl-phenyl)-6-propargyloxy-quinazolin-2-yl]-methanone;
[4-(4-Isopropyl-phenyl)-6-propargyloxy-quinazolin-2-yl]-thiazol-5-yl-methanone;
[4-(4-Isopropyl-phenyl)-6-propargyloxy-quinazolin-2-yl]-piperidin-1-yl-methanone;
4-(4-Isopropyl-phenyl)-6-prop-2-ynyloxy-quinazoline-2-carboxylic acid (3-chloro-phenyl)-amide;
4-(4-Isopropyl-phenyl)-6-prop-2-ynyloxy-quinazoline-2-carboxylic acid (3-methoxy-phenyl)-amide;
4-(4-Isopropyl-phenyl)-6-prop-2-ynyloxy-3,4-dihydro-quinazoline-2-carboxylic acid (3-methylsulfanyl-phenyl)-amide;
4-(4-Isopropyl-phenyl)-6-prop-2-ynyloxy-3,4-dihydro-quinazoline-2-carboxylic acid (3-methanesulfonyl-phenyl)-amide;
4-(4-Isopropyl-phenyl)-6-prop-2-ynyloxy-quinazoline-2-carboxylic acid (3-trifluoromethylsulfanyl-phenyl)-amide;
4-(4-Isopropyl-phenyl)-6-prop-2-ynyloxy-3,4-dihydro-quinazoline-2-carboxylic acid (3-sulfamoyl-phenyl)-amide;
4-(4-Isopropyl-phenyl)-6-prop-2-ynyloxy-3,4-dihydro-quinazoline-2-carboxylic acid [3-(2-hydroxy-ethanesulfonyl)-phenyl]-amide;
4-(4-Isopropyl-phenyl)-6-prop-2-ynyloxy-3,4-dihydro-quinazoline-2-carboxylic acid (5-ethanesulfonyl-2-hydroxy-phenyl)-amide;
4-(4-Isopropyl-phenyl)-6-prop-2-ynyloxy-quinazoline-2-carboxylic acid (3-nitro-phenyl)-amide;
4-(4-Isopropyl-phenyl)-6-prop-2-ynyloxy-quinazoline-2-carboxylic acid (3-cyano-phenyl)-amide;
3-{[4-(4-Isopropyl-phenyl)-6-prop-2-ynyloxy-quinazoline-2-carbonyl]-amino}-benzoic acid methyl ester;
3-{[4-(4-Isopropyl-phenyl)-6-prop-2-ynyloxy-quinazoline-2-carbonyl]-amino}-benzoic acid ethyl ester;
3-{[4-(4-Isopropyl-phenyl)-6-prop-2-ynyloxy-quinazoline-2-carbonyl]-amino}-benzoic acid isopropyl ester;
3-{[4-(4-Isopropyl-phenyl)-6-prop-2-ynyloxy-quinazoline-2-carbonyl]-amino}-benzoic acid tert-butyl ester;
4-(4-Isopropyl-phenyl)-6-prop-2-ynyloxy-quinazoline-2-carboxylic acid (3-carbamoyl-phenyl)-amide;
4-(4-Isopropyl-phenyl)-6-prop-2-ynyloxy-quinazoline-2-carboxylic acid (3-acetyl-phenyl)-amide;
3-{[4-(4-Isopropyl-phenyl)-6-prop-2-ynyloxy-quinazoline-2-carbonyl]-amino}-5-methoxy-benzoic acid methyl ester;
4-(4-Isopropyl-phenyl)-6-prop-2-ynyloxy-quinazoline-2-carboxylic acid (3-methylcarbamoyl-phenyl)-amide;
4-(4-Isopropyl-phenyl)-6-prop-2-ynyloxy-quinazoline-2-carboxylic acid (3-tert-butylcarbamoyl-phenyl)-amide;
4-(4-Isopropyl-phenyl)-6-prop-2-ynyloxy-quinazoline-2-carboxylic acid (3-dimethylcarbamoyl-5-trifluoromethyl-phenyl)-amide;
3-{[4-(4-Isopropyl-phenyl)-6-prop-2-ynyloxy-quinazoline-2-carbonyl]-amino}-5-trifluoromethyl-benzoic acid methyl ester;
3-{[4-(4-Isopropyl-phenyl)-6-prop-2-ynyloxy-quinazoline-2-carbonyl]-amino}-5-trifluoromethyl-benzoic acid isopropyl ester;
2-Fluoro-5-{[4-(4-isopropyl-phenyl)-6-prop-2-ynyloxy-quinazoline-2-carbonyl]-amino}-benzoic acid methyl ester;
2-Fluoro-5-{[4-(4-isopropyl-phenyl)-6-prop-2-ynyloxy-quinazoline-2-carbonyl]-amino}-benzoic acid isopropyl ester;
2-Chloro-5-{[4-(4-isopropyl-phenyl)-6-prop-2-ynyloxy-quinazoline-2-carbonyl]-amino}-benzoic acid methyl ester;
2,5-Dichloro-3-{[4-(4-isopropyl-phenyl)-6-prop-2-ynyloxy-quinazoline-2-carbonyl]-amino}-benzoic acid methyl ester;
2,5-Dichloro-3-{[4-(4-isopropyl-phenyl)-6-prop-2-ynyloxy-quinazoline-2-carbonyl]-amino}-benzoic acid isopropyl ester;
4-(4-Isopropyl-phenyl)-6-prop-2-ynyloxy-quinazoline-2-carboxylic acid (3-cyano-5-fluoro-phenyl)-amide;
4-(4-Isopropyl-phenyl)-6-prop-2-ynyloxy-quinazoline-2-carboxylic acid (3,4-dicyano-phenyl)-amide;
4-(4-Isopropyl-phenyl)-6-prop-2-ynyloxy-quinazoline-2-carboxylic acid (4-cyano-3-trifluoromethyl-phenyl)-amide;
4-(4-Isopropyl-phenyl)-6-prop-2-ynyloxy-quinazoline-2-carboxylic acid (3-trifluoromethyl-phenyl)-amide;
4-(4-Isopropyl-phenyl)-6-prop-2-ynyloxy-quinazoline-2-carboxylic acid (4-acetylamino-3-trifluoromethyl-phenyl)-amide;
4-(4-Isopropyl-phenyl)-6-prop-2-ynyloxy-quinazoline-2-carboxylic acid (3-methoxy-5-trifluoromethyl-phenyl)-amide;
4-(4-Isopropyl-phenyl)-6-prop-2-ynyloxy-quinazoline-2-carboxylic acid (3,5-bis-trifluoromethyl-phenyl)-amide;
4-(4-Isopropyl-phenyl)-6-prop-2-ynyloxy-quinazoline-2-carboxylic acid (3-fluoro-5-trifluoromethyl-phenyl)-amide;
4-(4-Isopropyl-phenyl)-6-prop-2-ynyloxy-quinazoline-2-carboxylic acid (4-fluoro-3-trifluoromethyl-phenyl)-amide;
3-{[4-(4-Isopropyl-phenyl)-6-prop-2-ynyloxy-quinazoline-2-carbonyl]-amino}-2-methyl-benzoic acid methyl ester;
3-{[4-(4-Isopropyl-phenyl)-6-prop-2-ynyloxy-quinazoline-2-carbonyl]-amino}-4-methyl-benzoic acid methyl ester;
3-{[4-(4-Isopropyl-phenyl)-6-prop-2-ynyloxy-quinazoline-2-carbonyl]-amino}-4-methoxy-benzoic acid methyl ester;
5-{[4-(4-Isopropyl-phenyl)-6-prop-2-ynyloxy-quinazoline-2-carbonyl]-amino}-isophthalic acid dimethyl ester;
4-{[4-(4-Isopropyl-phenyl)-6-prop-2-ynyloxy-quinazoline-2-carbonyl]-amino}-phthalic acid dimethyl ester;
4-(4-Isopropyl-phenyl)-6-prop-2-ynyloxy-quinazoline-2-carboxylic acid (3,5-dichloro-phenyl)-amide;
4-(4-Isopropyl-phenyl)-6-prop-2-ynyloxy-quinazoline-2-carboxylic acid (3,4-dichloro-phenyl)-amide;
4-(4-Isopropyl-phenyl)-6-prop-2-ynyloxy-quinazoline-2-carboxylic acid (3-chloro-4-fluoro-phenyl)-amide;
4-(4-Isopropyl-phenyl)-6-prop-2-ynyloxy-quinazoline-2-carboxylic acid (4-chloro-3-trifluoromethyl-phenyl)-amide;
5-{[4-(4-Isopropyl-phenyl)-6-prop-2-ynyloxy-quinazoline-2-carbonyl]-amino}-pyridine-2-carboxylic acid methyl ester;
5-{[4-(4-Isopropyl-phenyl)-6-prop-2-ynyloxy-quinazoline-2-carbonyl]-amino}-nicotinic acid methyl ester;
5-{[4-(4-Isopropyl-phenyl)-6-prop-2-ynyloxy-quinazoline-2-carbonyl]-amino}-nicotinic acid isopropyl ester;
[4-(4-Isopropyl-phenyl)-6-prop-2-ynyloxy-quinazolin-2-yl]-pyrrol-1-yl-methanone;
4-(4-Isopropyl-phenyl)-6-prop-2-ynyloxy-quinazoline-2-carboxylic acid (5-methyl-1H-pyrazol-3-yl)-amide;
(2-{[4-(4-Isopropyl-phenyl)-6-prop-2-ynyloxy-quinazoline-2-carbonyl]-amino}-thiazol-4-yl)-acetic acid ethyl ester;
4-(4-Isopropyl-phenyl)-6-prop-2-ynyloxy-quinazoline-2-carboxylic acid naphthalen-1-ylamide;
4-(4-Isopropyl-phenyl)-6-prop-2-ynyloxy-quinazoline-2-carboxylic acid isoquinolin-8-ylamide;
4-(4-Isopropyl-phenyl)-6-prop-2-ynyloxy-quinazoline-2-carboxylic acid phthalazin-5-ylamide;
4-(4-Isopropyl-phenyl)-6-prop-2-ynyloxy-quinazoline-2-carboxylic acid quinolin-5-ylamide;
4-(4-Isopropyl-phenyl)-6-prop-2-ynyloxy-quinazoline-2-carboxylic acid quinolin-8-ylamide;
4-(4-Isopropyl-phenyl)-6-prop-2-ynyloxy-quinazoline-2-carboxylic acid isoquinolin-4-ylamide;
4-(4-Isopropyl-phenyl)-6-prop-2-ynyloxy-quinazoline-2-carboxylic acid (5-acetyl-quinolin-8-yl)-amide;
4-(4-Isopropyl-phenyl)-6-prop-2-ynyloxy-quinazoline-2-carboxylic acid (3-bromo-6-methoxy-quinolin-8-yl)-amide;
4-(4-Isopropyl-phenyl)-6-prop-2-ynyloxy-quinazoline-2-carboxylic acid quinolin-2-ylamide;
4-(4-Isopropyl-phenyl)-6-prop-2-ynyloxy-quinazoline-2-carboxylic acid quinolin-6-ylamide;
4-(4-Isopropyl-phenyl)-6-prop-2-ynyloxy-quinazoline-2-carboxylic acid (2-methyl-quinolin-6-yl)-amide;
(6-{[4-(4-Isopropyl-phenyl)-6-prop-2-ynyloxy-quinazoline-2-carbonyl]-amino}-quinolin-8-yloxy)-acetic acid ethyl ester;
4-(4-Isopropyl-phenyl)-6-prop-2-ynyloxy-quinazoline-2-carboxylic acid (1H-benzoimidazol-4-yl)-amide;
4-(4-Isopropyl-phenyl)-6-prop-2-ynyloxy-quinazoline-2-carboxylic acid benzothiazol-2-ylamide;
4-(4-Isopropyl-phenyl)-6-prop-2-ynyloxy-quinazoline-2-carboxylic acid (benzo[1,3]dioxol-5-ylmethyl)-amide;
4-(4-Isopropyl-phenyl)-6-prop-2-ynyloxy-quinazoline-2-carboxylic acid (thiophen-2-ylmethyl)-amide;
4-(4-Isopropyl-phenyl)-6-prop-2-ynyloxy-quinazoline-2-carboxylic acid 3-methoxy-phenyl ester;
1-(4-Isopropyl-phenyl)-7-prop-2-ynyloxy-isoquinoline-3-carboxylic acid ethyl ester;
1-(4-Isopropyl-phenyl)-7-prop-2-ynyloxy-isoquinoline-3-carboxylic acid 1,2-dimethyl-propyl ester;
1-(4-Isopropyl-phenyl)-7-prop-2-ynyloxy-isoquinoline-3-carboxylic acid isobutyl ester;
1-(4-Isopropyl-phenyl)-7-prop-2-ynyloxy-isoquinoline-3-carboxylic acid cyclopropylmethyl ester;
1-(4-Isopropyl-phenyl)-7-prop-2-ynyloxy-isoquinoline-3-carboxylic acid benzyl ester;
1-(4-Isopropyl-phenyl)-7-prop-2-ynyloxy-isoquinoline-3-carboxylic acid 2-methoxy-benzyl ester;
1-(4-Isopropyl-phenyl)-7-prop-2-ynyloxy-isoquinoline-3-carboxylic acid 3-methoxy-benzyl ester;
1-(4-Isopropyl-phenyl)-7-prop-2-ynyloxy-isoquinoline-3-carboxylic acid 4-methoxycarbonyl-benzyl ester;
1-(4-Isopropyl-phenyl)-7-prop-2-ynyloxy-isoquinoline-3-carboxylic acid phenethyl ester;
1-(4-Isopropyl-phenyl)-7-prop-2-ynyloxy-isoquinoline-3-carboxylic acid 1-phenyl-ethyl ester;
1-(4-Isopropyl-phenyl)-7-prop-2-ynyloxy-isoquinoline-3-carboxylic acid [3-(2-hydroxy-ethanesulfonyl)-phenyl]-amide;
[1-(4-Isopropyl-phenyl)-7-prop-2-ynyloxy-isoquinolin-3-yl]-(3-methoxy-phenyl)-methanone; and
[1-(4-Isopropyl-phenyl)-7-prop-2-ynyloxy-isoquinolin-3-yl]-(4-methoxy-phenyl)-methanone;

8. A pharmaceutical composition comprising a compound of claim 1 in association with a pharmaceutically acceptable excipient, diluent or carrier.

9. (canceled)

10. A method for preventing or treating bone conditions which are associated with increased calcium depletion or resorption or in which stimulation of bone formation and calcium fixation in the bone is desirable in which an effective amount of a compound of claim 1 or a pharmaceutically-acceptable and -cleavable ester, or acid addition salt thereof is administered to a patient in need of such treatment.

11. A process for preparation of a compound of formula (I) in free or salt form, comprising the step of:
(i) for cases where Q is N, reacting a compound of formula (III) with a compound of formula (IV) and an ammonium salt in the presence of a suitable solvent: 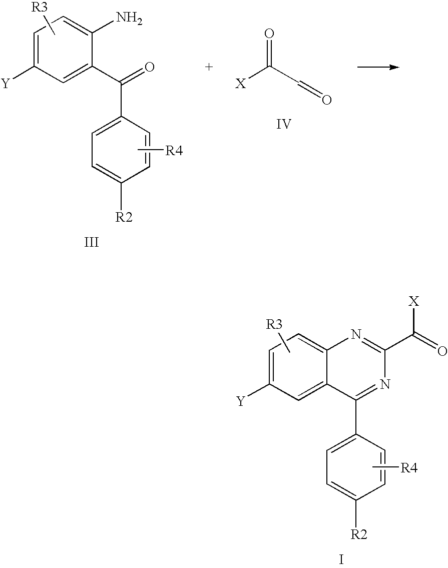
or
(ii) reacting a compound of formula V 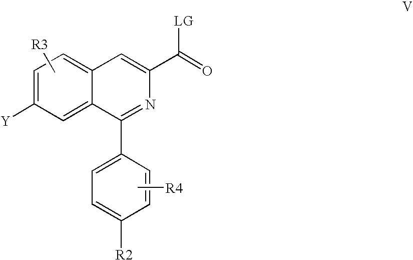
wherein LG represents a suitable leaving group;
with an organometallic reagent of formula VI:
X-Met VI
under suitable anhydrous conditions; or
(iii) reacting a compound of formula Va 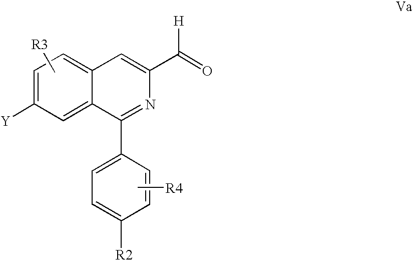
with an organometallic reagent of formula VI:
X-Met VI
under suitable anhydrous conditions followed by oxidation to the carbonyl compound by an appropriate oxidation agent; or
(iv) reacting a compound of formula VII 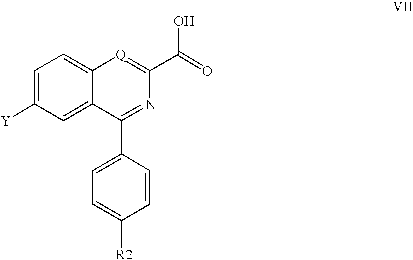
with a compound X—H wherein the H forms part of an amino or hydroxy group, the reaction being carried out in the presence of a coupling reagent; or
(v) reacting a compound of formula VIII 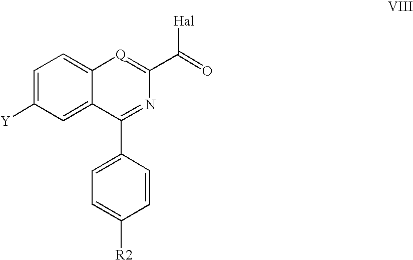
wherein Hal is halogen or a leaving group
with a compound X—H wherein the H forms part of an amino or hydroxy group, the reaction being carried out in the presence of a coupling reagent.

12. (canceled)

13. (canceled)

14. A compound according to claim 4 wherein R2′ is isopropyl, t-butyl or cyclopropyl.

15. A compound according to claim 3 wherein X′ is optionally substituted (aryl, heteroaryl, heterocycloalkyl, arylamino, heteroarylamino, aryl C₁-C₄ alkylamino, heteroaryl C₁-C₄ alkylamino, aryloxy, heteroaryloxy, C-C6 alkyloxy, aryl C₁-C₄ alkyloxy, heteroaryl C₁-C₄ alkyloxy).

16. A pharmaceutical composition of claim 8 further comprising a second substance selected from: calcium, a calcitonin or an analogue or derivative thereof, a steroid hormone, a partial estrogen agonist or estrogen-gestagen combination, a SERM (Selective Estrogen Receptor Modulator), a RANKL antibody, a cathepsis K inhibitor, vitamin D or an analogue thereof or PTH, a PTH fragment or a PTH derivative for simultaneous, separate or sequential treatment.
